(19) United States
(12) Patent Application Publication
(10) Pub. No.: US 2004/0053274 A1
(43) Pub. Date: Mar. 18, 2004

(54) Gene cluster of vicenistatin biosynthesis, a vicenisamine glycosyltransferase polypeptide, and a gene encoding the polypeptide

(75) Inventor: Katsumi Kakinuma, Kawasaki City (JP)

Correspondence Address: BURNS DOANE SWECKER & MATHIS L L P, POST OFFICE BOX 1404, ALEXANDRIA, VA, 22313-1404, US

(73) Assignee: Tokyo Institute of Technology, Tokyo (JP)

(21) Appl. No.: 10/378,083

(22) Filed: Mar. 4, 2003

(30) Foreign Application Priority Data
Sep. 4, 2002 (JP) 2002-258,838

(51) Int. Cl. C12Q 1/68; C07H 21/04; C12P 21/06; C12N 9/10

(52) U.S. Cl. 435/6; 435/69.1; 435/193; 435/320.1; 435/325; 435/68.1; 536/23.2

(57) ABSTRACT

To achieve enzymatic production of vicenistatin, a valuable compound, this invention provides enzymes involved in biosynthesis of vicenistatin and genes encoding the enzymes. This invention provides base sequence of the vin cluster which encodes enzyme complex responsible for biosynthesis of vicenistatin. This invention further provides a glycosyltransferase (VinC enzyme), which catalyzes production of vicenistatin using dTDP-vicenisamine as a substrate. Moreover, this invention provides a gene (vinC gene) encoding the enzyme. The VinC enzyme is useful for enzymatic synthesis of valuable compound vicenistatin. Furthermore, it is suggested that use of the VinC enzyme will enable development of novel polyketide glycosides.

### BACKGROUND OF THE INVENTION

1. Field of the Invention

This invention relates to an enzyme cluster of vicenistatin biosynthesis enzyme, and a gene cluster of vicenistatin biosynthesis enzyme encoding the enzyme cluster. Particularly, this invention relates to VinC enzyme, which is a glycosyltransferase catalyzing the reaction to produce vicenistatin using dTDP-vicenisamine as a substrate, and to vinC gene encoding the enzyme.

2. Prior Art

Enthusiastic investigation on genes encoding biosynthesis of polyketide compounds has been performed worldwide, and attempts to create new polyketides have been also performed by deletion or insertion of the gene. Under such situation, to obtain novel polyketide compounds, not only simple polyketides but also complex polyketides have attracted attention of researches as novel genetic resources, and wide-range investigation has been performed.

Vicenistatin is an anticancer antibiotic produced by Streptomyces sp. HC-34 strain of actinomycetes. From a study on experimental human cancer research using nude mice which received transplantation of human cancer cells, it has been known that vicenistatin can markedly inhibit growth of human intestinal cancer cells. As to its chemical structure, plane structure of vicenistatin and absolute conformation on vicenisamine (sugar moiety of vicenistatin) were firstly elucidated. Then, the absolute configuration of the aglycon was determined. The actinomycetes species capable of producing vicenistatin was identified as Streptomyces halstedii HC-34.

As to the biosynthetic pathway of vicenistatin, it was elucidated that it consists of the following steps. Firstly, glutamate as a starting material undergoes a series of reactions including mutase reaction to produce a polyketide starter unit. Then, propionate and acetate are introduced as precursors to evoke a normal polyketide elongation reaction to produce a characteristic 20-membered cyclic aglycon portion. It was clarified that vicenisamine was derived from glucose via conversion of functional groups similar to reaction involved in production of other aminosugars, and N-methyl groups derived from methionine was incorporated for biosynthesis of vicenisamine.

To create a novel compound by further modification on the molecular structure through biosynthetic engineering, attempts have been made for identification of genes responsible for biosynthesis of vicenistatin. A number of novel lactam polyketide antibiotics were discovered by researches mainly achieved in Japan, and some studies were reported on biosynthetic precursors. However, no study has been made on the genes and enzymes involved in the biosynthesis of those compounds.

### SUMMARY OF THE INVENTION

Vicenistatin has a structure comprising unique 20-membered lactam glycoside and it is a noteworthy anti biotic because of its strong inhibitory effect on growth of human solid cancer cells transplanted into experimental models. However, vicenistatin is not put into practical use yet because of its limited solubility and moderate toxicity. However, if these disadvantages will be conquered in the future by researches on chemical or biosynthetic engineering, it will provide a useful medicine. Therefore, for the purpose to achieve development of novel useful substances by biosynthetic engineering, elucidation and modification on the system involved in biosynthesis of the unique chemical structure are required. Thus, the object of this invention is to obtain genes involved in biosynthesis of vicenistatin, which is the principle genes in the biosynthetic system, and to elucidate functions of the genes.

Vicenistatin is a glycoside containing an aminosugar. There is no known compound containing sugar moiety identical to vicenisamine, but it was assumed that some similar genes responsible for biosynthesis of similar aminosugars might be involved in biosynthesis of vicenistatin. It is known that the aglycon portion is a polyketide derived from acetate/propionate as its precursor. From this knowledge, it was predicted that genes similar to those responsible for biosynthesis of polyketide synthases (PKS) were involved in biosynthesis of the aglycon portion. However, it was also known that the polyketide starter unit comprises unique amino acids derived from glutamate, therefore, it was predicted that certain novel genes different from those conventionally known might be involved in biosynthesis of this polyketides.

In this invention, an attempt was made to identify genes responsible for biosynthesis of vicenistatin, using knowledge on ketosynthases (KS), which is indispensable for biosynthesis of polyketides and having high homology with synthases of various polyketides, as well as analogies of aminosugars and singular starter unit, using techniques of PCR and hybridization. As a result, the genes encoding cluster of enzymes indispensable to produce the ultimate structure of vicenistatin were identified and base sequences of the genes were sequenced. This knowledge provides a great possibility for modification of vicenistatin biosynthesis through biosynthetic engineering, by methods such as modification, deletion or duplication in the base sequence of the biosynthetic genes, or insertion of some exogenous genes, etc.

The first aspect of this invention is to provide a gene cluster comprising a base sequence represented by base Nos. 1-64492 shown in the SEQ ID No. 1 in the sequence listing.

The second aspect of this invention is to provide a polypeptide comprising an amino acid sequence represented by amino acid Nos. 1-419 shown in the SEQ ID No. 4 in the sequence listing. A polypeptide comprising an amino acid sequence in which a part of the amino acid sequence is deleted or another amino acid sequence is added to the amino acid sequence, or a part of the amino acid sequence is substituted with another amino acid sequence, and exhibiting enzymatic activity as a glycosyltransferase, is also included within the scope of this invention. Furthermore, a gene encoding the polypeptide is also included within the scope of this invention.

The third aspect of this invention is to provide a gene comprising a base sequence represented by base Nos. 2790-4049 shown in the SEQ ID No. 1 in the sequence listing. A gene comprising a base sequence in which a part of the base sequence is deleted or another base sequence is added to the base sequence, or a part of the base sequence is substituted with another base sequence, and encoding a polypeptide exhibiting enzymatic activity as a glycosyltransferase, is also included within the scope of this invention.

The fourth aspect of this invention is to provide a method for production of vicenistatin by catalytic reaction of the glycosyltransferase comprising the polypeptide as described above, using dTDP-vicenisamine as a starting material.

### BRIEF DESCRIPTION OF THE DRAWINGS

FIG. 1 is a schematic figure showing gene mapping of the gene obtained in this study.

FIG. 2 is a figure showing results of homology analysis performed on the respective open reading frames (ORFs).

FIG. 3 is a figure showing the biosynthetic pathway of vicenistatin agylcon.

FIG. 4 is a figure showing the biosynthetic pathway of vicenistatin.

FIG. 5 is a figure showing the synthetic pathway of the substrates (dTDP-vicenisamine, dTDP-mycarose, and dTDP-2-deoxyglucose) used for the enzyme reaction.

### DETAILED DESCRIPTION OF THE INVENTION

A cosmid library comprising DNA insert fragments of about 40 kbp were constructed according to the conventional method, from the genome of Streptomyces halstedii sp. HC-34, which is a vecenistatin (an antibiotic) producing bacteria. From knowledge on biosynthesis of aminosugar, it was assumed that both of dTDP-glucose-4,6-dehydratase and dTDP-4-keto-6-deoxyglucose-2,3-dehydratase were involved in biosynthesis of vicenisamine, which is a sugar component of vecenistatin. Therefore, the known base sequences of the enzymes were used to design probes and investigations were performed using PCR and other approaches. Consequently, two kinds of cosmids were isolated.

Subsequently, the two kinds of cosmids were sequenced. In general, genes involved in biosynthesis of antibiotics form a cluster and exist in forms of one mass. In view of this knowledge, the inventors searched for which is the gene encoding the enzyme concerned here. In this case, the previously reported knowledge on the mechanism of biosynthesis of antibiotics was effective. Particularly, in view of the knowledge on the sequence conserved among genes encoding ketosynthase (KS) involved in polyketide biosynthesis, the inventors limited candidate genes responsible for vicenistatin biosynthesis enzyme to cosmids K1B10 and K1D11. They analyzed on base sequence of the genes existing adjacent to these cosmids, and functions of respective open reading frames (ORFs) were predicted from homology search from existing databases.

Again, the previous observation was-effective, that is, it was known that the starter unit of the lactam portion of vicenistatin was derived from glutamate and the starter unit was formed via a mutase reaction. Based on this knowledge, the inventors found ORFs having high homology with known glutamate mutase. Moreover, they confirmed that putative function of respective modules in the ORFs highly correlated with the biosynthetic pathway of vicenistatin. The entire cluster and the respective open reading frames of the sugar portion and the lactam portion were named to be vin gene, in connection with the name of vicenistatin.

Furthermore, to confirm the function of the vin gene cluster, the inventors expressed glycosyltransferase gene (vinC) in E. coli, and the glycosyltransferase was used for the reaction between chemically synthesized dTDP-vicenisamine and the aglycon chemically derived from vicenistatin. As a result, it was confirmed that this vinC efficiently catalyzed this reaction as a transferase. Therefore, it was confirmed that the vin gene cluster was responsible for vicenistatin biosynthesis. Hitherto, the gene identified to be a biosynthetic gene of macrocyclic lactam antibiotic was limited to only rifamycin, which is a member of ansamycin group antibiotics. Rifamycin is different from vicenistatin in various aspects including its chemical structure and biological activity, therefore, the gene cluster of this invention will serve as a valuable resource for further creation of novel substances, using techniques of biosynthetic engineering.

As to vinC gene which function was identified in this invention, the VinC protein was produced in a large scale in E. coli, by the technique of electrophoresis. In addition, to confirm its function, glycosyltransferase reaction was performed between chemically synthesized dTDP-vicenisamine and the vicenistatin aglycon. Moreover, it was detected that the VinC enzyme can catalyze transferase reaction of chemically synthesized dTDP-mycarose or dTDP-2-deoxyglucose with the vicenistatin aglycon. From this result, it was suggested that use of the enzyme of this invention would enable synthesis of a novel polyketide glycosides hitherto unknown. According to this invention, genes responsible for biosynthesis of vicenistatin (an anticancer antibiotic) were identified. Among these enzymes, particularly, the function of VinC was confirmed as a glycosyltransferase.

The base sequence described in SEQ ID No.1 in the sequence listing represents the gene corresponding to the entire region of the gene cluster of vicenistatin biosynthesis enzyme, obtained by analysis of the base sequence of the region of cosmid K1B10 and the regions adjacent to it. Genes involved in biosynthesis of vicenistatin form a cluster and the genes described in SEQ ID No.1 in the sequence listing encode various enzymes involved in vicenistatin biosynthesis, not only the glycosyltransferase.

That is, in the gene described in SEQ ID No.1 in the sequence listing, the portion corresponding to base Nos. 2790-4049 encodes a glycosyltransferase, serving for biosynthesis of vicenistatin using vicenisamine as a substrate. The transferase of vicenisamine is a polypeptide represented by an amino acid sequence as listed in the SEQ ID No.4 in the sequence listing.

The polypeptide consisting of an amino acid sequence in which a part of said polypeptide represented by amino acid sequence shown in SEQ ID NO: 4 in the sequence listing is deleted, substituted or added with another amino acid sequence means a polypeptide in which ten or less, preferably five or less, and more preferably two or less amino acids of the sequence is deleted, substituted or added to the amino acid sequence represented by SEQ ID NO: 4 in the sequence listing. Moreover, such polypeptide exhibits homology 90% or more, preferably 95% or more and still preferably 99% or more with the amino acid sequence represented by in SEQ ID NO: 4 in the sequence listing. Such polypeptide is also within the scope of this invention so far as it exhibits enzymatic activity as a glycosyltransferase, that is, catalyzing synthesis of vicenistatin using vicenisamine as a substrate.

According to technique of gene recombination, artificial modification can be achieved at a specific site of basic DNA, without alteration or with improvement of basic characteristic of said DNA. Concerning a gene having native sequence provided according to this invention or modified sequence different from said native sequence, it is also possible to perform artificial modification such as insertion, deletion or substitution to obtain gene of equivalent or improved characteristic compared with said native gene. Moreover, a gene with such mutation is also included in the scope of this invention.

That is, the gene, consisting of a base sequence in which a part of said gene represented by base Nos. 2790-4049 in the SEQ ID No.1 in the sequence listing is deleted, substituted or added with another base sequence means a gene in which ten or less, preferably five or less, and more preferably two or less bases of the sequence is deleted, substituted or added to the base sequence base Nos. 2790-4049 shown in the SEQ ID No.1 in the sequence listing. Moreover, such gene exhibits homology 90% or more, preferably 95% or more and still preferably 99% or more with the base sequence represented by base Nos. 2790-4049 shown in the SEQ ID No. 1 in the sequence listing. Such gene is also within the scope of this invention so far as it encodes a polypeptide exhibiting enzymatic activity as a glycosyltransferase, that is, catalyzing synthesis of vicenistatin using vicenisamine as a substrate. In addition, such gene hybridizes with the base sequence represented by base Nos. 2790-4049 shown in the SEQ ID No.1 in the sequence listing under stringent conditions.

The condition for hybridization can be selected by those skilled in the art ad libitum. That is, hybridization can be performed by the following procedure. DNA molecules or RNA molecules to be tested are transferred onto a membrane, then the membrane is hybridized with a labeled probe in a proper hybridization buffer. The hybridization buffer may comprise, for example, 5×SSC, 5% (weight %) dextran sulfate, 1/20 volume of blocking reagent and 2 to 7×SSC, however, other various formulation can be also used for the hybridization buffer. The blocking reagent for nucleic acid hybridization may comprise, for example, a buffer (pH 7.5) containing 0.1M maleic acid and 0.15M sodium chloride, and commercially available blocking reagent for hybridization may be dissolved into the buffer at the concentration of 10%. The 20×SSC solution may comprise 3M sodium chloride and 0.3M citrate, and the SSC solution may be preferably utilized at the concentration of 3 to 6×SSC, more preferably at the concentration of 4 to 5×SSC.

The temperature for hybridization may preferably be 40 to 80° C., more preferably be 50 to 70° C., further more preferably be 55 to 65° C. Incubation may be performed from several hours to overnight, then washed by a washing buffer. The temperature for washing may preferably be room temperature, more preferably it may be the temperature used for hybridization. The formulation for the washing buffer may preferably comprise 6×SSC and 0.1% (weight %) SDS, more preferably may comprise 4×SSC and 0.1% (weight %) SDS, further preferably may comprise 2×SSC and 0.1% (weight %) SDS, more further preferably may comprise 1×SSC and 0.1% (weight %) SDS, most preferably may comprise 0.1×SSC and 0.1% (weight %) SDS. The membrane may be washed by such washing buffer, then DNA molecule or RNA molecule may be distinguished by the hybridization with the labeled probe.

It is known that the gene cluster shown in the sequence ID No. 1 of the sequence listing includes plural ORFs, except for that of the glycosyltransferase, and predicted amino acid sequences encoded by the ORFs are also obtained. Moreover, the functions of the enzymes defined by these amino acid sequences can be predicted based on the results of homology search with amino acid sequences derived from various enzymes.

In the SEQ ID No. 1 in the sequence listing, bases Nos. 695-1762 represents an ORF and the gene corresponding to the ORF (vinA) encodes a protein consisting of 355 amino acids. The sequence shown in SEQ ID No. 2 in the sequence listing represents predicted amino acid sequence encoded by the vinA gene. As will be detailed later, that protein is assumed to be dTDP-glucose synthase.

In the SEQ ID No. 1 in the sequence listing, bases Nos. 1759-2730 represents an ORF and the gene corresponding to the ORF (vinB) encodes a protein consisting of 323 amino acids. The sequence shown in SEQ ID No. 3 in the sequence listing represents predicted amino acid sequence encoded by the vinB gene. As will be detailed later, that protein is assumed to be dTDP-glucose 4,6-dehydratase.

In the SEQ ID No. 1 in the sequence listing, bases Nos. 4412-11194 represents an ORF and the gene corresponding to the ORF (vinP2) encodes a protein consisting of 2260 amino acids. The sequence shown in SEQ ID No. 5 in the sequence listing represents predicted amino acid sequence encoded by the vinP2 gene. As will be detailed later, that protein is assumed to be polyketide synthase module.

In the SEQ ID No. 1 in the sequence listing, bases Nos. 11260-21348 represents an ORF and the gene corresponding to the ORF (vinP3) encodes a protein consisting of 3362 amino acids. The sequence shown in SEQ ID No. 6 in the sequence listing represents predicted amino acid sequence encoded by the vinP3 gene. As will be detailed later, that protein is assumed to be polyketide synthase module.

In the SEQ ID No. 1 in the sequence listing, bases Nos. 21411-32837 represents an ORF and the gene corresponding to the ORF (vinP4) encodes a protein consisting of 3808 amino acids. The sequence shown in SEQ ID No. 7 in the sequence listing represents predicted amino acid sequence encoded by the vinP4 gene. As will be detailed later, that protein is assumed to be polyketide synthase module.

In the SEQ ID No. 1 in the sequence listing, bases Nos. 33262-33972 (complementary strand) represents an ORF and the gene corresponding to the ORF (vinG) encodes a protein consisting of 236 amino acids. The sequence shown in SEQ ID No. 8 in the sequence listing represents predicted amino acid sequence encoded by the vinG gene. As will be detailed later, that protein is assumed to be N-methyltransferase, that is, an enzyme closely related to dTDP-N-dimethyldesosamine-N-methyltransferase.

In the SEQ ID No. 1 in the sequence listing, bases Nos. 33980-35173 (complementary strand) represents an ORF and the gene corresponding to the ORF (vinF) encodes a protein consisting of 397 amino acids. The sequence shown in SEQ ID No. 9 in the sequence listing represents predicted amino acid sequence encoded by the vinF gene. As will be detailed later, that protein is assumed to be dTDP-hexose-aminotransferase.

In the SEQ ID No. 1 in the sequence listing, bases Nos. 35320-36801 represents an ORF and the gene corresponding to the ORF (vinD) encodes a protein consisting of 493 amino acids. The sequence shown in SEQ ID No. 10 in the sequence listing represents predicted amino acid sequence encoded by the vinD gene. As will be detailed later, that protein is assumed to be dTDP-4-keto-6-deoxyglucose-2,3-dehydratase.

In the SEQ ID No. 1 in the sequence listing, bases Nos. 36849-37841 represents an ORF and the gene corresponding to the ORF (vinE) encodes a protein consisting of 330 amino acids. The sequence shown in SEQ ID No. 11 in the sequence listing represents predicted amino acid sequence encoded by the vinE gene. As will be detailed later, that protein is assumed to be dTDP-4-keto-6-deoxyhexose-2,3-reductase.

In the SEQ ID No. 1 in the sequence listing, bases Nos. 38022-38498 represents an ORF and the gene corresponding to the ORF (vinH) encodes a protein consisting of 158 amino acids. The sequence shown in SEQ ID No. 12 in the sequence listing represents predicted amino acid sequence encoded by the vinH gene. As will be detailed later, that protein is assumed to be S-subunit of glutamate mutase.

In the SEQ ID No. 1 in the sequence listing, bases Nos. 38495-39904 represents an ORF and the gene corresponding to the ORF (vinI) encodes a protein consisting of 469 amino acids. The sequence shown in SEQ ID No. 13 in the sequence listing represents predicted amino acid sequence encoded by the vinI gene. As will be detailed later, that protein is assumed to be E-subunit of glutamate mutase.

In the SEQ ID No. 1 in the sequence listing, bases Nos. 39990-40889 represents an ORF and the gene corresponding to the ORF (vinJ) encodes a protein consisting of 299 amino acids. The sequence shown in SEQ ID No. 14 in the sequence listing represents predicted amino acid sequence encoded by the vinJ gene. As will be detailed later, that protein is assumed to be proline iminopeptidase.

In the SEQ ID No. 1 in the sequence listing, bases Nos. 40886-41869 represents an ORF and the gene corresponding to the ORF (vinK) encodes a protein consisting of 327 amino acids. The sequence shown in SEQ ID No. 15 in the sequence listing represents predicted amino acid sequence encoded by the vinK gene. As will be detailed later, that protein is assumed to be acyl CoA-ACP transacylase closely related to malonyl CoA-ACP transacylase.

In the SEQ ID No. 1 in the sequence listing, bases Nos. 41987-42235 represents an ORF and the gene corresponding to the ORF (vinL) encodes a protein consisting of 82 amino acids. The sequence shown in SEQ ID No. 16 in the sequence listing represents predicted amino acid sequence encoded by the vinL gene. As will be detailed later, that protein is assumed to be acyl carrier protein.

In the SEQ ID No. 1 in the sequence listing, bases Nos. 42289-43863 represents an ORF and the gene corresponding to the ORF (vinM) encodes a protein consisting of 524 amino acids. The sequence shown in SEQ ID No. 17 in the sequence listing represents predicted amino acid sequence encoded by the vinM gene. As will be detailed later, that protein is assumed to be non-ribosomal type peptide synthase.

In the SEQ ID No. 1 in the sequence listing, bases Nos. 44156-45592 represents an ORF and the gene corresponding to the ORF (vinN) encodes a protein consisting of 478 amino acids. The sequence shown in SEQ ID No. 18 in the sequence listing represents predicted amino acid sequence encoded by the vinN gene. As will be detailed later, that protein is assumed to be long chain fatty acid-CoA ligase.

In the SEQ ID No. 1 in the sequence listing, bases Nos. 45589-46833 represents an ORF and the gene corresponding to the ORF (vinO) encodes a protein consisting of 414 amino acids. The sequence shown in SEQ ID No. 19 in the sequence listing represents predicted amino acid sequence encoded by the vinO gene. As will be detailed later, that protein is assumed to be PLP-dependent decarboxylase.

In the SEQ ID No. 1 in the sequence listing, bases Nos. 46982-64492 represents an ORF and the gene corresponding to the ORF (vinP1) encodes a protein consisting of 5836 amino acids. The sequence shown in SEQ ID No. 20 in the sequence listing represents predicted amino acid sequence encoded by the vinP1 gene. As will be detailed later, that protein is assumed to be polyketide synthase module.

The vin gene cluster is consisted of these genes and the proteins encoded by the gene cluster are involved in vicenistatin biosynthesis. Information on this gene cluster is useful for efficient production of vicenistatin, as well as for tools available for development of novel antibiotics. Therefore, this invention provides a great opportunity for development of novel useful substances.

### EXAMPLES

### Example 1

### Screening of 4,6-dehydratase Gene

Deoxysugars are ubiquitously present in bacterial systems as a constituent of the cell wall or of secondary metabolites. Indeed, deoxysugars are present in many kinds of antibiotics such as macrolides and anthracylines. List of known deoxysugars include, for example, mycarose and desosamine contained in erythromycin, daunosamine contained in daunorubicin, mikamynose contained in tyrosine, and vancosamine contained in vancomycin, etc. Many deoxysugars play an important role in exerting the activity of those antibiotics.

Recently, many studies have been made on biosynthesis of 2,6-deoxysugars, and the biosynthesis of various deoxysugars is generally believed to proceed as follows: glucose-1-phosphate is metabolized to NDP-glucose by NDP-glucose synthase; the latter is then deoxygenated at C-6 by NDP-glucose-4,6-dehydratase to yield NDP-4-keto-6-deoxy glucose; and it is further deoxygenated at C-2 by NDP-4-keto-6-deoxy glucose 2,3-dehydratase. This sequence is the common pathway of 2,6-deoxysugar biosynthesis, and various 2,6-deoxysugars are assumed to be biosynthesized as the result of further modification.

Many genes involved in biosynthesis of various deoxysugars have been cloned. In biosynthetic pathway of 2,6-deoxysugars, it was demonstrated that the genes coding for 4,6-dehydratase and 2,3-dehydratase revealed to exhibit high homology with respective congeners. Thus they serve as a powerful tool for cloning of genes involved in the production of the secondary metabolite products.

Based on a study using labeled precursors, it was confirmed that vicenisamine was derived from glucose. Therefore, it was predicted that vicenisamine would also take the same biosynthetic pathway similar to other 2,6-deoxysugars. Thus, the inventors presumed that a gene responsible for biosynthesis of vicenistatin would be cloned by utilizing a gene responsible for biosynthesis of deoxysugar as a probe. Thus, the inventors utilized a gene coding for 4,6-dehyratase as a probe for screening on a genomic library, because the 4,6-dehyratase gene exhibited high homology among corresponding genes.

(Design of Primers)

Gene coding for 4,6-dehydratase exhibited very high homology among corresponding genes. In the base sequences of 4,6-dehydratase, the regions of 46DH-1 and 46DH-2 were known to be conserved in their sequences. It was expected that if sense and anti-sense primers were designed based on the base sequences of 46DH-1 and 46DH-2 and the two primers were used in PCR, a DNA fragment of 530 bp would be amplified.

46DH-1: 5′-ACSGGYCSBGCCGCHTTCATCGG-3′

46DH-2: 5′-GRWRCTGRTRSGGCCGTAGTTGTT-3′

(where S represents C or G; Y represents C or T; B represents C, G or T; H represents A, C or T; R represents A or G; and W represents A or T.)

(PCR Using Genomic DNA as a Template)

To perform screening on genomic library directed to isolation of clones containing a gene coding for 4,6-dehydratase, it is necessary to confirm whether 4,6-dehydratase gene is specifically amplified using the designed primers. At first, PCR was conducted using a genome of Streptomyces halstedii HC34 (S. halstedii) as a template, for this purpose. PCR condition consisted of 30 cycles with a cycle of 94° C. for 7 minutes, 94° C. for 30 seconds, 52° C. for 30 seconds, and 72° C. for 60 seconds, which was lastly followed by 72° C. for 7 minutes. As a result of the PCR, a DNA fragment having an expected size (about 530 bp) was obtained.

(Sequencing of the PCR Product)

The DNA fragment obtained by PCR was subcloned into a pT7Blue T vector, which was then introduced into E. coli. Plasmids were isolated on the obtained transformants and the sequences were confirmed to obtain three sequences of 46DH-A, 46DH-B and 46DH-C. The inventors performed homology search on the databases, and found that these sequences commonly exhibited high homology with known 4,6-dehydratase genes. From this, it was confirmed that the designed primers enabled the specific amplification of the gene encoding 4,6-dehydratase and that they could be used for screening of genomic library in the search of the target gene. It was predicted that any one of the three sequences was obtained by amplifying a gene involved in biosynthesis of vicenistatin. Next, a genomic library was screened using 46DH-1 and 46DH-2 as primers.

(Screening of the Genomic Library)

A genomic library derived from S. halstedii HC34 was constructed as follows. A genomic library is a population of DNA clones obtained by cutting the chromosomal DNA of a living organism into fragments having an appropriate length, and subcloning of these fragments into a vector. Each DNA fragment comprises a part of the genome chosen at random. To prepare a genomic library of S. halstedii HC34, the genome of S. halstedii HC34 was randomly digested with Sau3AI, and DNA fragments having a length of approximately 40 kbp were subcloned into a cosmid vector pOJ446, which gave a total population of 6048 clones. They were subdivided into 63 pools of 96 clones and they were stored as the divided 63 pools, otherwise the target gene has to be cloned from enormous amount of 6048 clones.

(Primary Screening)

Primary screening by PCR was conducted on the 63 pools in order to screen pools containing a gene encoding 4,6-dehydratase. Using a library fraction contained in each pool as a template, PCR was conducted with 46DH-1 and 46DH-2 as primers, and pools exhibiting amplification of DNA fragment of the purpose having a size of 530 bp were selected as a positive candidate. Thus, 30 pools were found to be positive.

In the same manner, PCR primers of 23DH-1 and 23DH-2 were designed from dTDP-4-keto-6-deoxyglucose 2,3-dehydratase gene, which was known to have high homology among cognate genes, and PCR based on the primers was conducted using the genome as a template. As a consequence, two kinds of base sequences (23DH-A and 23DH-B) were confirmed and both of which exhibited high homology with known 2,3-dehydratase genes. When the library was screened using these primers, 17 positive clones were obtained. The sequences of 23DH-1 and 23DH-2 are as shown below.

| | |
|---|---|
| 23DH-1: | 5′-AGGCCACCCGSAGCAACTACAC-3′ |
| 23DH-2: | 5′-GAASCGSCCGCCCTCCTCSGA-3′ |

In general, genes involved in biosynthesis of secondary metabolites in microorganisms tends to form a cluster. Therefore, it was expected that the genes encoding 4,6-dehydratase and 2,3-dehydratase, both of which are involved in the synthesis of vicenistatin, would exist close to each other, and thus the two genes of the purpose might exist in a same cosmid. Therefore, nine pools containing the two genes were selected as positive candidates.

(Secondary Screening)

PCR for the secondary screening proceeded in the same manner as in the first screening. In the secondary screening, those containing two genes of 4,6-dehydratase gene and 2,3-dehydratase gene were selected as positive. As a consequence, two cosmid clones (K1B10 and K1D11) were isolated.

(Classification of Positive Clones)

As described above, the two cosmids of K1B10 and K1D11 contained 4,6-dehydratase gene and 2,3-dehydratase gene. Based on sequence result of the PCR products, it was demonstrated that there are three kinds of genes (46DH-A, 46DH-B and 46DH-C) and two kinds of genes (23DH-A and 23DH-B) coding for 4,6-dehydratase and 2,3-dehydratase respectively, existing on the genome. Thus, the inventors confirmed on what kind of 4,6-dehydratase and 2,3-dehydratase correspond to those contained in the K1B10 and the K1D11, respectively. For this purpose, the inventors performed PCR using each cosmid as a template, PCR products were subcloned into an appropriate vector, then the clones were sequenced. As a result, they found that 4,6-dehydratase gene and 2,3-dehydratase gene contained in the K1B10 correspond to 46DH-A and 23DH-A, respectively. In the same manner it was revealed that 4,6-dehydratase gene and 2,3-dehydratase gene contained in the K1D11 correspond to 46DH-B and 23DH-B, respectively.

From this, it was suggested that the two cosmids represented different portions of the genome. It had been known that there were two kinds of genes coding for 2,3-dehydratase in the genome, and cosmids containing respective 2,3-dehydratase genes were cloned. Therefore, it was expected that genes responsible for vicenistatin biosynthesis were likely to exist either in the cosmid K1B10 or in the cosmid K1D11. Thus, the inventors proceeded to the analysis of the two cosmids.

(Estimation on the Genes Contained in the Positive Clones)

The two cosmids contained different kinds of genes coding for 4,6-dehydratase and 2,3-dehydratase, thus, it was assumed that the genes were involved in the biosynthesis of two different 2,6-deoxysugars. It was suggested that one of the 2,6-deoxysugars was vicenisamine, and that regions adjacent to 4,6-dehydratase gene and 2,3-dehydratase gene in the cosmids K1B10 and K1D11 might contain other genes coding for the enzymes involved in biosynthesis of vicenisamine. The inventors tried to confirm which one of the two cosmids contained genes encoding for enzyme catalyzing biosynthesis of the deoxysugar and involved in biosynthetic system of vicenistatin.

Each cosmid had been prepared to have an insert size of about 40 kbp. Thus, it was not easy to sequence the total insert at one time. Therefore, an attempt was made to estimate what gene is contained in each of the cosmid. For this purpose, the cosmid was digested with restriction enzymes, and the resulting DNA fragments were subcloned into an appropriate vector, then the clones were sequenced.

Firstly, the cosmid K1B10 was cleaved with BamHI, BglII and EcoRI, the resulting DNA fragments were subcloned. The cosmid K1B10 was found to contain base sequences having high homologies with those coding for NDP-glucose 4,6-dehydratase, NDP-4-keto-6-deoxyglucose 2,3-dehydratase, NDP-hexose aminotransferase, and NDP-aminohexose-N-methyltransferase. The cosmid also contained sequences having high homologies with those for PKS (polyketide synthase) module I and glutamate mutase. On the other hand, the cosmid K1D10 was found to contain base sequences having high homologies with those for PKS module I and gycosyltransferase. It further contained a sequence having high homology with that for cytochrome P450.

Comparison of the genes contained in the two cosmids revealed that the cosmid K1B10 contained the genes coding for aminotransferase and for methyltransferase, both of which were considered to be involved in the biosynthesis of vicenisamine, as well as the gene for glutamate mutase which was predicted to be involved in biosynthesis of vicenistatin starter. From this it was suggested that genes existing in the region adjacent to the cosmid K1B10 were involved in the biosynthesis of vicenistatin.

### Example 2

### Sequencing of Gene Cluster

Then, regions in and adjacent to the cosmid K1B10 were sequenced to obtain the entire sequence involved in biosynthesis of vicenistatin. Cosmid clones adjacent to the cosmid K1B10 were obtained, and the clones were sequenced. The base sequence data obtained were translated into amino acid sequences, and their functions of the respective genes were estimated by performing homology search based on public database (BLAST).

(Sequencing of the Cosmid)

A DNA sequencer reveals, at each run, the sequence of a DNA fragment having a length of about 600 to 700 bp at maximum. The cosmids derived from genomic library of S. halstedii HC34 was produced to contain the insert having length of about 40 kbp. In order to sequence the insert having such a large size, it was necessary to cut the cosmid DNA into fragments having an appropriate size, and to subclone the fragments into an appropriate vector. If the cosmid DNA was cut into small fragments having a length of about 500 bp, the base sequence of the both chains could be sequenced. However, in such case, identification of the restriction sites would be very difficult and ligation of respective fragments would become an excessive work. To cope with this, each insert of 40 kbp was cleaved with an appropriately chosen restriction enzyme into fragments of about 2 to 10 kbp; the fragments were subcloned into a vector; the clones were sequenced; and the thus obtained sequences were ligated properly.

An EZ::TN<KAN-2> insertion kit enables random insertion of a transposon into a plasmid. By using primers specific to both ends of a transposon, sequence can be determined on the regions adjacent to the inserted transposon.

Then under the presence of both of template plasmid DNA and transposon, the transposon is inserted in by transposase, and E. coli is transformed by the plasmid DNA. Since kanamycin-resistance gene is encoded on the transposon, it is assumed that the transformants containing template plasmid containing the inserted transposon may exhibit resistance to kanamycin. Therefore, transformants of the purpose can be selected based on the kanamycin-resistance. The transformants containing the plasmid are cultivated, and the sequence was determined using the purified plasmid as a template. This method is appropriate for analyzing a DNA fragment having a length of about 2 to 10 kbp. Thus, the inventors confirmed overall sequences of the respective fragments obtained by restriction enzyme treatment of K1B10, and ligated these fragments.

(Acquisition of Cosmids Adjacent to K1B10)

The sequence data of the K1B10 revealed that both ends of its insert contained base sequences having high homologies with TDP-glucose synthase and glutamate mutase. These enzymes were likely to be involved in biosynthesis of vicenistatin, and it was expected that gene cluster involved in vicenistatin biosynthesis would be extended to outside of this region.

Thus, an attempt was made to obtain other cosmid clones having regions adjacent to the K1B10. Firstly, an attempt was made to obtain cosmids containing left region of the K1B10. Therefore, based on the base sequence of K1B10C1 existing left end of the K1B10C1 insert, specific PCR primers (K1B10-leftF, K1B10-leftR) were designed, and genomic library was screened again by PCR using these primers. Primary and secondary screenings were performed as above to obtain cosmid Y3D4. Furthermore, in the same manner with the left end, PCR primers designed for the right end of the cosmid K1B10 based on the base sequence of K1B10AS (K1B1O-rightF, K1B10-rightR), and they were used for screening and cosmid K7G3 was obtained. Further primers were designed based on the K7G3 and screened, and cosmid L6D5 was obtained.

### Example 3

### Homology Analysis of ORFs

Base sequence was determined on the approximately 60 bp region existing adjacent to the cosmid K1B10. When a base sequence is translated into amino acid sequence, six different frames can be given in total, that is, three frames for the forward direction and three frames for the reverse direction, respectively. In general, a prokaryote has a region of about 5 bp with abundant A (adenine) and G (guanine), existing about 10 bp upstream from the start codon, and this region is called Shine-Dalgarno (SD) sequence. It is known that ribosome binds to this region to initiate translation. Therefore, based on this knowledge, the inventors analyzed on ORFs encoding for amino acid sequences.

However, as to actinomycetes, it is known that the SD sequence does not necessarily exist in the organisms. Therefore, an alternative method is known, that is, the frame can be analyzed by calculating GC content in the frame. The chromosomal DNA of actynomycetes usually exhibits high GC content of 70% or more in the average. However, contents of amino acids corresponding to codons comprising only GC, namely arginine, glycine, proline and alanine, are not significantly higher compared with those of other prokaryotes. The third base of a triplet codon is not practically important to determine amino acid encoded by the codon, and GC content in the third base of the triplet codon is extremely high (90% or higher), while GC content for the second base is very low (about 50%) and that for the first base is about 70%. Moreover, as to region not translated to corresponding amino acid, such as promoter, GC content in the region is about 50%. For example, GC content is analyzed on the 5 kbp region existing left end of the K1B10 using a computer program of Frame Plot. As a result, four ORFs (orfA, orfB, orfC and orfP2) were revealed to exist in the region.

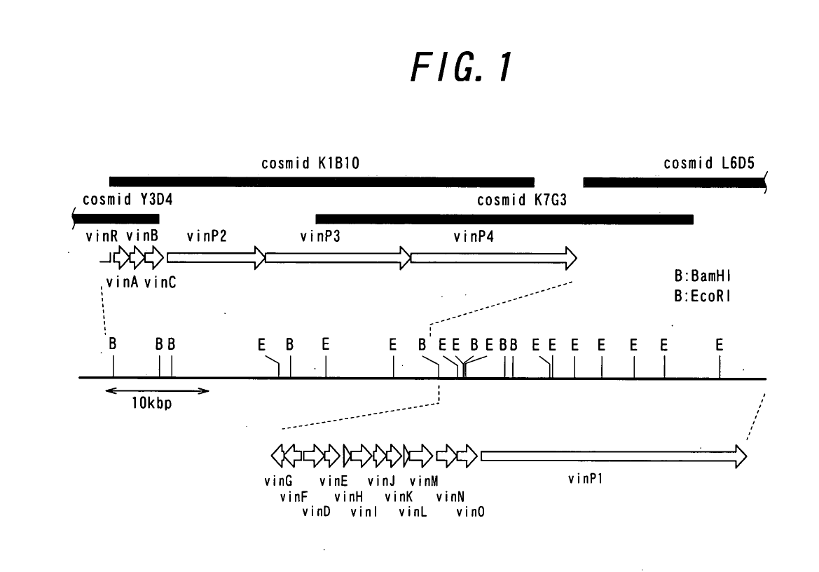

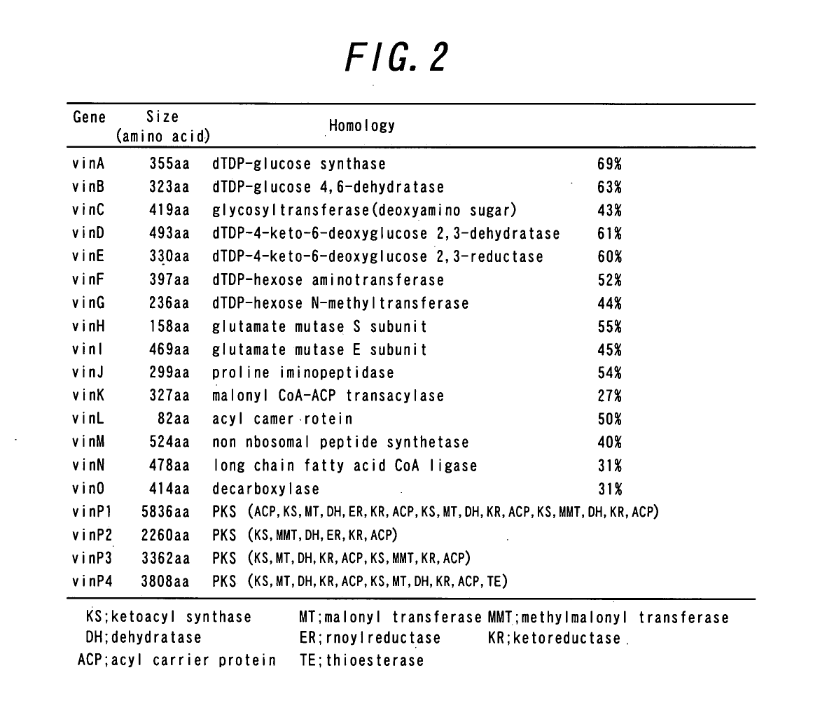

In the same manner, frame analysis was performed on the all regions to confirm existence of 18 ORFs. FIG. 1 shows a gene mapping obtained in the present experiment. Homology search was performed for the respective ORFs by public databases (BLAST), based on its putative amino acid sequence. Proteins revealed the highest homologies were listed in FIG. 2.

(Homology Analysis of orfA)

The orfA consisted of 1068 bp, and was estimated to represent a protein comprising 355 aa (amino acids, that is, the number of amino acid residues) having a molecular weight of about 38 kDa. The orfA corresponds with the vinA gene described above. As a result of the search through databases (BLAST), the protein encoded by the orfA exhibited 69% homology with MtmD (protein ID T48866: glucose-1-phosphate thymidylyl transferase), which is contained in gene cluster of S. argillaceus, and involved in biosynthesis of mithramycin. Moreover, the protein encoded by the orfA exhibited 67% homology with StrD (protein ID A26984: glucose-1-phosphate thymidylyl transferase) of S. griseus which is involved in biosynthesis of streptomycin. Besides, it exhibited high homologies with dTDP-glucose synthases of various secondary metabolites.

(Homology Analysis of orfB)

The orfB consisted of 972 bp, and was estimated to represent a protein comprising 323 aa having a molecular weight of about 36 kDa. The orfB corresponds with the vinB gene described above. As a result of database search, the protein encoded by the orfB exhibited high homology with dTDP-glucose-4,6-dehydratase of various Streptomyces species. This enzyme is involved in deoxygenation of a sugar at C-6.

The orfC consisted of 1260 bp, and was estimated to represent a protein comprising 419 aa having a molecular weight of about 46 kDa. The orfC corresponds with the vinC gene described above. As a result of database search, the protein encoded by the orfC exhibited high homology with various glycosyltransferases.

All of the proteins exhibited high homology with the orfC are glycosyltransferase of 2,6-deoxysugars involved in biosynthesis of antibiotics. It is known that these enzymes contain conserved amino acid sequences of DXD and LPXCXXhhHHGGXGXXXhAhbXGhPQbbP (where X represents an arbitrary amino acid residue and h represents a hydrophobic amino acid residue). The protein encoded by the orfC also contained these sequences, and from this it was estimated that the protein would be a glycosyltransferase of 2,6-deoxysugars. Furthermore, as the protein had high homology with glycosyltransferase of aminosugars (particularly among 2,6-deoxysugars), it was suggested that the protein serves as glycosyltransferase in the biosynthesis of vicenistatin.

(Homology Analysis of orfD)

The orfD consisted of 1482 bp, and was estimated to represent a protein comprising 493 aa having molecular weight of about 55 kDa. The orfD corresponds with the vinD gene described above. As a result of database search, the protein encoded by the orfD exhibited high homology with known dTDP-4-keto-6-deoxyglucose 2,3-dehydratase. For example, the protein exhibited 61% homology with SnogH (protein ID CAA12009: estimated 2,3-dehydratase) derived from S. nogalater).

(Homology Analysis of orfE)

The orfE consisted of 993 bp, and was estimated to represent a protein comprising 330 aa having molecular weight of about 36 kDa. The orfE corresponds with the vinE gene described above. As a result of database search, the protein encoded by the orfE exhibited high homology with known dTDP-4-keto-6-deoxyhexose 2,3-reductase, i.e. it exhibited 60% homology with TylCVI (protein ID AAD41821, S. fradiae). This enzyme is involved in reduction of sugar at C-3 position, and is believed to be also involved in the reduction of sugar at C-3 position in vicenistatin biosynthesis.

(Homology Analysis of orfF)

The orfF consisted of 1194 bp, and was estimated to represent a protein comprising 397 aa having molecular weight of about 43 kDa. The orfF corresponds with the vinF gene described above. As a result of database search, the protein encoded by the orfF exhibited high homology with aminotransferases involved in biosynthesis of various aminosugars. For example, the protein exhibited 48% homology with EryCIV (protein ID CAA72084) aminotransferase involved in biosynthesis of desosamine, which is an aminosugar constituting erythromycin produced by Saccharopolyspora erythraea, and exhibited 37% homology with VioA (protein ID AAD44154) involved in biosynthesis of 4-amino-2,6-dideoxyglucose produced by E. coli. From these facts it was estimated that the orfF is an enzyme participating in amino group introduction in vicenistatin biosynthesis.

(Homology Analysis of orfG)

The orfG consisted of 711 bp, and was estimated to represent a protein comprising 236 aa having molecular weight of about 26 kDa. The orfG corresponds with the vinG gene described above. As a result of database search, the protein encoded by the orfG exhibited 45% homology with methyltransferases utilizing S-adenosylmethionine as the methyl group donor, e.g., the protein exhibited 45% homology with methyltransferase AknX2 (protein ID AAF73460) from S. gallilaeus, and the protein exhibited 44% homology with methyltransferase EryCVI (protein ID CAA72082), which is involved in biosynthesis of desosamine and derived from Saccharopolyspora erythraea. From a study using labeled methionine, it is known that the N-methyl group of vicenisamine is derived from S-adenosylmethionine. From these observations, it was estimated that the protein encoded by the orfG is methyltransferase involved in vicenisamine biosynthesis.

(Homology Analysis of orfH and orfI)

The orfH consisted of 477 bp, and was estimated to represent a protein comprising 158 aa having molecular weight of about 17 kDa. The orfI consisted of 1410 bp, and was estimated to represent a protein comprising 469 aa having molecular weight of about 50 kDa. The orfH corresponds with the vinH gene described above while the orfI corresponds with the vinI gene described above. As a result of databases search, the protein encoded by the orfH exhibited high homology with S-subunit of glutamate mutase, while the protein encoded by the orfI exhibited high homology with E-subunit of glutamate mutase. Glutaminate mutase is a unique enzyme in that it isomerises glutamate into 3-methylaspartate utilizing vitamin B12 (adenosylcobalamin) as a coenzyme.

X-ray crystallographic analysis on site-directed mutated enzymes was performed on GlmE, S (accession number, X80997) of Clostridium cochlearium. The study revealed that His16 of the S-subunit serves as a binding site for the coenzyme, and that Glu171 of the E-subunit serves to recognize the amino group of glutamic acid. In addition, these amino acids are preserved in the protein encoded by the orfI. It has been known that glutamate and 3-methylaspartate are produced as intermediates in vicenistatin biosynthesis, as described above. This suggests that the proteins encoded by the orfH and orfI may catalyze the aforementioned reaction.

(Homology Analysis of orfJ) The orfJ consisted of 900 bp, and was estimated to represent a protein comprising 299 aa having molecular weight of about 34 kDa. The orfJ corresponds with the vinJ gene described above. As a result of database search, the protein encoded by the orfJ exhibited 54% homology with proline iminopeptidase from Mesorhizobium loti. Besides, it exhibited high homology with proline iminopeptidase from various microorganisms. Contribution of orfJ in vicenistatin biosynthesis could not be predicted only from the homology analysis. However, generally in microorganisms, genes coding for microbial secondary metabolites form a cluster, and thus it seemed possible that this protein might be involved in the biosynthesis of vicenistatin in some way. Gene disruption experiment of the orfJ will be requisite to elucidate the function of this protein in vicenistatin biosynthesis.

(Homology analysis of orfK)

The orfK consisted of 984 bp, and was estimated to represent a protein comprising 327 aa having molecular weight of about 36 kDa. The orfK corresponds with the vinK gene described above. As a result of database search, the protein encoded by orfK exhibited homology with acyl CoA-ACP (acyl carrier protein) transacylase of type-II fatty acid synthases derive from various species. When an amino acid serving as a starter unit in the biosynthesis of vicenistatin is incorporated into a polyketide synthase (PKS), this enzyme is considered to catalyze conversion of the CoA derivatives of amino acids into the corresponding ACP derivatives.

(Homology Analysis of orfL)

The orfL consisted of 249 bp, and was estimated to represent a protein comprising 82 aa having molecular weight of about 9.6 kDa. The orfL corresponds with the vinL gene described above. As a result of databases search, the protein encoded by the orfL exhibited homology with known ACP. This protein is considered to serve as an ACP to which a glutamate derivative binds, when the derivative is captured by PKS.

(Homology Analysis of orfM)

The orfM consisted of 1575 bp, and was estimated to represent a protein comprising 524 aa having molecular weight of about 56 kDa. The orfM corresponds with the vinM gene described above. As a result of database search, the protein encoded by orfM exhibited homology with adenylation domain A (A domain) of type I non-ribosomal peptide synthetase. It was considered that the protein could be involved in activation of amino acids in vicenistatin biosynthesis, that is, in isomerization of the amino acid at biosynthesis of the starter unit or in the formation of macrolactam. If the function of this enzyme is elucidated through further study utilizing gene disruption experiments and overexpression of the enzyme, it will provide interesting results.

(Homology Analysis of orfN)

The orfN consisted of 1437 bp, and was estimated to represent a protein comprising 478 aa having a molecular weight of about 52 kDa. The orfN corresponds with the vinN gene described above. As a result of database search, the protein encoded by the orfN was found to have homology with some CoA ligases, i.e., it exhibited 33% homology with estimated long chain fatty acid CoA ligase from Sinorhizobium meliloti, that is, SMb20650 (protein ID CAC49758). On the basis of a study using a labeled starter unit, it is believed that when 3-methylaspartic acid is captured by PKS, it undergoes decarboxylation and isomerization. The capture by PKS may involve the following steps: 3-methylaspartic acid is converted by CoA ligase into a CoA derivative which is then converted into an ACP derivative by the protein encoded by the orfK; and the ACP derivative is captured by PKS.

(Homology Analysis of orfO)

The orfO consisted of 1245 bp, and was estimated to represent a protein comprising 414 aa. The orfO corresponds with the vinO gene described above. As a result of database search, the protein encoded by the orfO was found to have a homology with decarboxylase utilized as a coenzyme pyridoxal-5′-phosphate, PLP. It had a highest homology (identity 31%, positive 51%) with BtrK (protein ID BAB18473, Bacillus circulans), which had been suggested to be involved in the biosynthesis of butirosin. Besides, the protein exhibited homology with diaminopimelate decarboxylase and ornithine decarboxylase, which had been believed to be involved in the primary metabolism. It was estimated from the level of homology that the protein encoded by the orfO is decarboxylase involved in the biosynthesis of secondary metabolites, but is rather different from decarboxylase involved in the primary metabolites. From the above results, it is suggested that the protein encoded by orfO may catalyze the expected decarboxylation of a starter unit in vicenistatin biosynthesis.

(Homology Analysis of orfs P1, P2, P3 and P4)

As a result homology analysis by database search, the proteins encoded by orfs P1, P2, P3 and P4 exhibited homology with type I PKSs. The orfs P1, P2, P3 and P4 correspond to previously described vinP1, vinP2, vinP3 and vinP4 genes, respectively. The amino acid sequences of the proteins were compared with other PKSs, and it was estimated that the orfP1 is comprised from three modules, orfP2 one module, orfP3 two modules, and orfP4 two modules.

Among functional domains of PKS, it is known that sequence analysis of AT domain enables to determine whether the extension unit is acetate or propionate. In malonyltransferase (MT) utilizing acetate as the extension unit, two motifs of XYAQXXXXXXQXAL and GHSI (where X represents an arbitrarily chosen amino acid residue) are preserved adjacent to its active site. On the other hand, in methylmalonyltransferase (MMT) utilizing propionate as the extension unit, two motifs of VDVVQXXXXXXMXSLA and GHSQ exist adjacent to the active site. The AT domains of the modules contained in the orfP1 included MT, MT and MMT, that in the orfP2 included MMT, those in the orfP3 included MT and MMT, and those in the orfP4 included MT and MT, in this order.

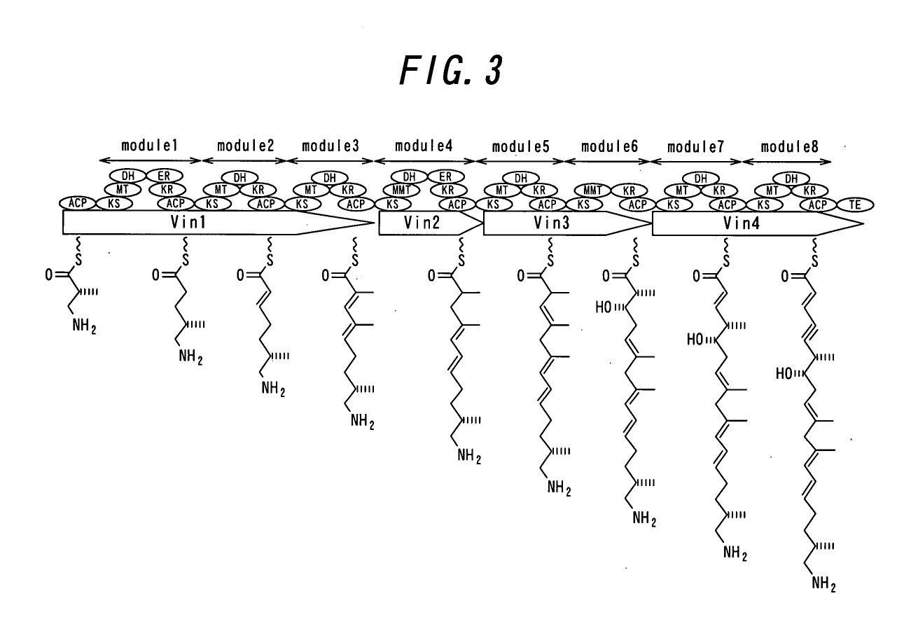

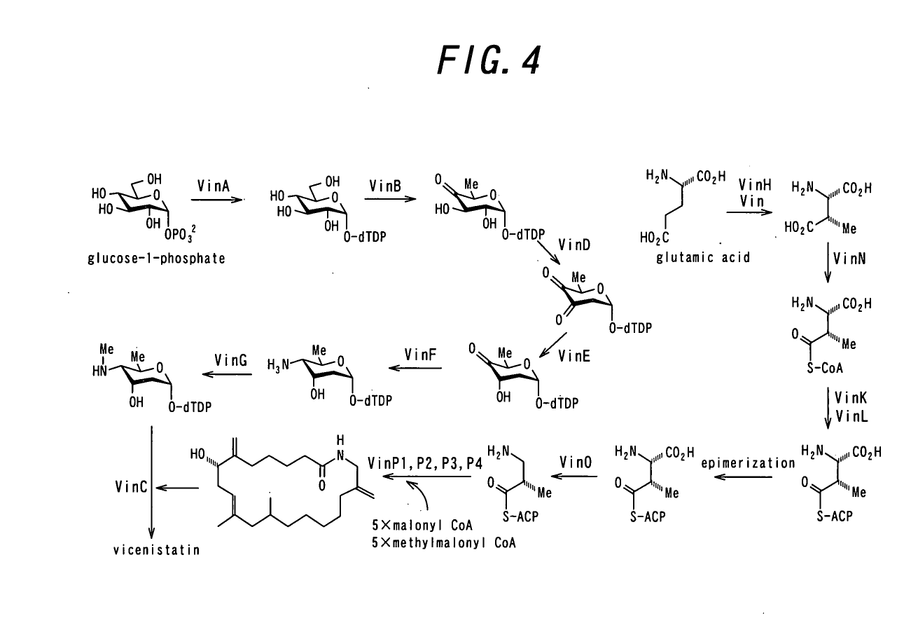

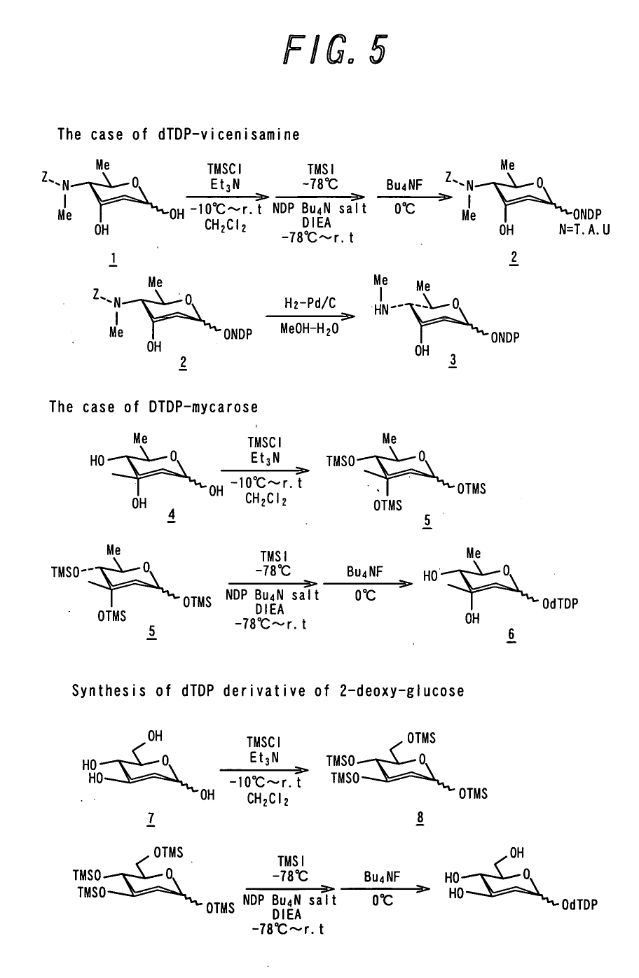

Assuming that the orfs P1, P2, P3 and P4 are extended in the order to exert their enzymatic action, the sequential order of the functional domain in the respective modules coincides with carbon backbone constituting aglycon of vicenistatin. Ultimately, the polyketide may be released from the enzyme by thioesterase (TE) domain existing on the OrfP4 to be converted into a ring (FIG. 3). This strongly suggests that the PKS genes concerned here are those responsible for the biosynthesis of vicenistatin. FIG. 4 summarizes the reactions catalyzed by the enzymes encoded by respective vin genes.

The base sequence analysis suggested that the proteins represented by orfs A, B, D, E, F and G are involved in the biosynthesis of vicenisamine, and that orfC encodes glycosyltransferase for deoxysugars. In addition, it suggested that the proteins represented by orfs H, I, K, L, N, O, P1, P2, P3, and P4 are involved in the biosynthesis of aglycon of vicenistatin. It was expected that these enzymes serve in this sequential order for biosynthesis of vicenistatin. Based on these observations, it was strongly suggested that the genes obtained through the above study should be responsible for the biosynthesis of vicenistatin. The predicted pathway of vicenistatin biosynthesis is summarized in the scheme shown in FIG. 4.

The proteins represented by 18 ORFs identified in this study did not include that possibly serving as isomerase for 3-methylaspartate, but any one enzyme bearing the activity may serve as an isomerase. Alternatively, the proteins represented by the Orf M and Orf J whose function could not be identified in this study might be involved in this function. Otherwise, the ORF concerned for this reaction might exist outside of the region analysed in this study.

### Example 4

### Expression of vinC by Recombinant E. coli

VinC was amplified from the cosmid K1B10 by PCR using two oligonucleotids (oligo EXPRESS SELECT), that is, vinC-N term (5′-AAGGTACCATATGCGCGTCCTGATGA-3′) and vinC-C term (5′-CCCGGATCCGCTGGGCGGAGTGCTAC-3′). The NdeI and BamHI sites inserted into the oligonucleotides are underlined. The used DNA polymerase was Takara Ex-Taq (Takara). The PCR conditions consisted of 30 cycles with a cycle of 95° C. for 7 minutes, 95° C. 30 for seconds, 56° C. for 30 seconds, and 72° C. for 90 seconds, followed by 72° C. for 7 minutes.

The PCR product was cloned into pT7Blue T vector; the sequence of the clones was confirmed and clones containing proper sequence were selected. VinC was cloned using NdeI and BamHI sites and cloned into pET30b(+) to produce pET-vinC. The obtained pET-vinC was used to transform E. coli BL21 (DE3), to produce recombinant E. coli cells for expression of vinC. The recombinant E. coli cells thus obtained were designated as BL21(DE3)/pETvinC. The procedures for conventional gene manipulation was performed in accordance with the description of “Molecular Cloning” written by Joseph Sambrook and David W. Russell, Cold Spring Harbor Laboratory Press.

### Example 5

### Confirmation on Enzymatic Activity of the Recombinant VinC

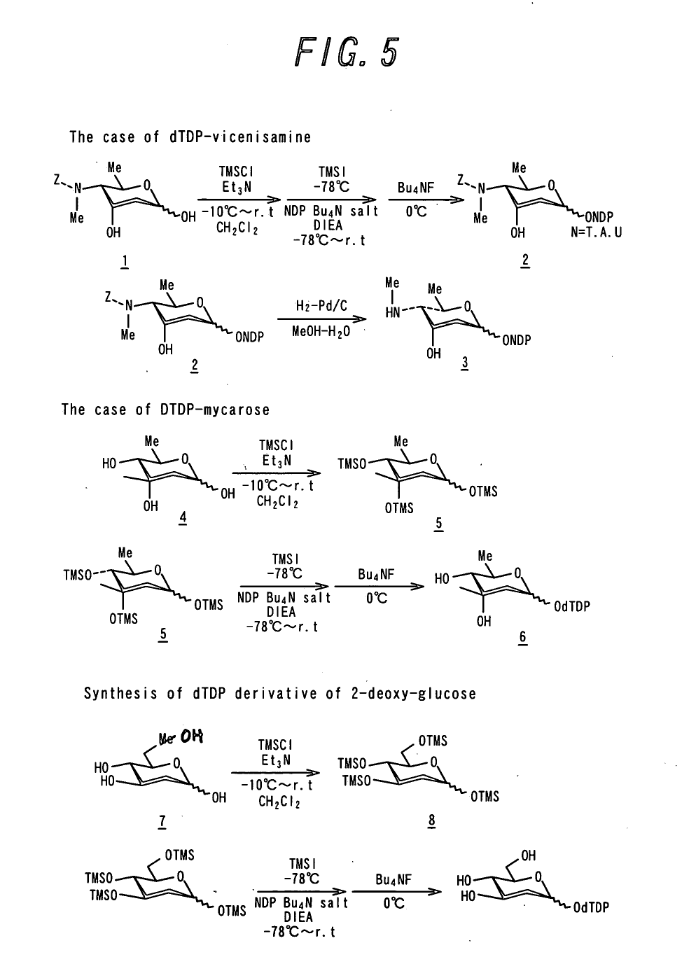

A cell-free extract was prepared from the cells of E. coli obtained as described for VinC expression and it was used as a crude enzyme solution. Then glycosyltransferase activity of the enzyme solution was confirmed using dTDP-vicenisamine as a substrate. The method to produce starting materials used in the enzyme reaction, and the result of the enzymatic activity will be described below. FIG. 5 shows the scheme for synthesis of dTDP-vicenisamine, dTDP-2-mycarose, and dTDP-2-deoxyglucose, used as substrates of the enzymatic reactions.

(Preparation of dTDP-Na Salt)

Sodium salt of dTDP (100 mg) was dissolved in cold water, and the solution was passed through a column filled with H⁺ type DOWEX AG-50 X8. Into the fraction containing the target substance having pH of 1 to 2, an aqueous solution of 4% tetrabutylammonium hydroxide (Bu₄N—OH) was added until the pH reached to 5 to 6. The solution was freeze-dried and a white crystalline substance was obtained.

(Synthesis of dTDP-vicenisamine)

The synthesized compound (1) (41.5 mg, 1.41×10⁻⁴ mol) was dissolved into 2 ml of CH₂Cl₂, and the solution was transferred into a bath containing acetone-dry ice and kept at −10° C. Into the solution, Et₃N (0.392 ml, 2.81×10⁻³ mol, 20 mol eq.) was added, and then TMS-Cl (0.213 ml, 1.68×10⁻³ mol, 12 mol eq.) was added. After stirring for 30 min, 0.4 ml of Et₃N and 0.2 ml of TMS-Cl were added. After another 30 min stirring, 0.2 ml of Et₃N and 0.1 ml of TMS-Cl were further added to the mixture. After stirring for 1.8 hour, the solution was concentrated with an evaporator.

To the residue, a solution of pentane/ethyl acetate (EA)=4 was added; the resulting solution was filtered and concentrated; and benzene was added to the solution followed by azeotropic evaporation, to give a solid. Then 1.2 ml of CH₂Cl₂ was added to the solid and it was kept at −78° C., to which was added TMS-I (0.026 ml, 1.83×⁻⁴ mol, 1.3 mol eq.). Forty-five minutes later, the resultant was added to a mixture containing dTDP-Bu₄N salt and 20 μl of diisopropylethylamine (DIEA) dissolved in 1.5 ml of CH₂Cl₂.

The solution was stirred for 1.5 hour, and then its temperature was gradually raised. After 1.3 hour, 120 μl of tetrabutylammonium fluoride (TBAF) was added to the solution. After 55 min of stirring, the solution was concentrated, and the concentrate was purified by reverse phase silica gel chromatography (water:methanol=1:1->1:5) to yield 103 mg of partially-purified sample. A solution of water:methanol=1:1 (7 ml in total) was added to the sample. To the solution, it was added with 11.3 mg of Pd/C, and the whole was subjected to hydrogenolysis in hydrogen atmosphere. After the reaction was completed, the mixture was filtered and concentrated. The concentrate was purified with a DEAE Sephadex column A-25 equillibated with 30 ml of NH₄HCO₃ solution to yield 13.2 mg (two steps, 16%) of dTDP-vicenisamine diammonium salt (compound (3)) in the form of white powder.

MS data: [C₁₇H₂₉N₃O₃P₂-H]-, and calculated MW: 544.11, measured MW: 544.2 (base peak)

Synthesis of ADP-vicenisamine and UDP-vicenisamine was performed by the process according to the procedure described above.

(Synthesis of dTDP-mycarose)

The compound (4) (46.4 mg, 2.86×10⁻⁴ mol) was added to 1 ml of pyridine, and the mixture was kept at 0° C., to which was added TMS-Cl (0.8 ml, 22 mol eq). After 11.5 hours, 7 ml of pentane was added, and cold water was added and then separated. It was washed until the residual pyridine becomes less than detection limit by TLC. The supernatant was concentrated, and the residue was used for further operation. The crude product (compound (5)) was dissolved in 1 ml of CH₂Cl₂, and the solution was kept at −78° C. Then, TMS-I (0.0529 ml, 1.3 mol eq.) was added thereto, and 45 minutes later, a mixture solution of dTDP-Bu₄N salt and 40 μl of DIEA dissolved in CH₂Cl₂ was added to it. After stirring for three hours, 480 μl of TBAF was added thereto, and the mixture was concentrated 40 minutes later. The concentrate was purified via DEAE-Sephadex as above to yield 21.5 mg of dTDP-mycarose (compound (6)).

(Synthesis of dTDP Derivative of 2-deoxyglucose)

2-Deoxyglucose (7)(31.3 mg, 1.91×10⁻⁴ mol) was dissolved into 1 ml of pyridine, to which was added TMS-Cl (0.5 ml, 3.94×10⁻³ mol, 20 mol eq.) at room temperature. After 1.5 hour, 5 ml of pentane was added, then cold water was added and separated. The supernatant was concentrated, and the concentrate was subjected to azeotropic treatment with benzene to afford a TMS derivative (compound (8)).

To this crude product was added 1 ml of CH₂Cl₂, and the mixture was cooled to −78° C. Then, TMS-I (0.3 ml, 2.48×10⁻⁴, 1.3 mol eq.) was added. Forty minutes later, a mixture (which was obtained by dissolving dTDP-Bu₄N salt and 40 μl of DIEA in CH₂Cl₂) was added. The temperature of the solution was gradually raised, and after stirring for 3.4 hours, 0.1 ml of TBAF was added to the solution. After additional stirring for fifty minutes, the solution was concentrated, and the concentrate was purified by DEAE Sephadex A-25 column.

(Enzyme Reaction)

The above-mentioned recombinant E. coli BL21(DE3)/pETvinC was used for pre-culture on 4 ml of LB medium containing kanamycin. Then, a mixture comprising 100 ml of LB medium, 30 μg/ml of kanamycin and 1 ml of the pre-culture was prepared and was incubated for two hours at 37° C. under shaking with 220 rpm. The cultivation continued until OD600 reached to about 0.6. IPTG was added to the culture until the concentration reached to 0.2 mM. The culture was further incubated at 37° C. for about three hours under shaking with 180 rpm. About 400 mg of the recombinant E. coli cells were collected, and a buffer (comprising 50 mM tris-HCl, pH 7.5; 1 mM, MnCl₂; 1 mM, MgCl₂) was added to the cell pellet, at such a volume corresponding to 10 times as much as that of the cell pellet (4 ml of buffer to 400 mg of cell pellet). The cell suspension was sonicated 10 times for two minutes to disrupt the cells. The resultant solution was centrifuged at 12,000 rpm for 30 minutes. The supernatant (cell-free extract) was collected and used as a crude enzyme solution.

A 14 μl of dTDP-vicenisamine dissolved in distilled water (3.3 mg/700 μl) and 3.6 μl of aglycon (saturated solution in DMSO) were transferred into an Eppendorf tube in an ice bath, and 100 μl of the crude enzyme solution was added thereto, and the mixture was stirred to allow the enzyme reaction to proceed. Twenty-four hours later, distilled EA and 2N NaOH were added to adjust pH of the solution at pH10 or higher. Then, the solution was extracted with ethyl acetate. The extracted supernatant was concentrated with an evaporator, and 30 μl of methanol was added thereto. In the same manner, dTDP-mycarose and dTDP-2-deoxyglucose were allowed to react separately with the aglycon.

The reaction product was detected with an LC-MS equipment (LCQ type, Finnigan) connected with a HPLC system equipping ODS column (Mightysil RP-18 GP, Kanto Kagaku). The reaction product was detected by UV 254 nm, and its structure was simultaneously analyzed by mass spectrometry. As a result, it was confirmed that the reaction between dTDP-vicenisamine and the aglycon in the presence of the enzyme underwent glycosyl transfer reaction to produce vicenistatin.

Mass spectral data: [M+H]⁺; calculated MW: 501.36; measured MW: 501.2

In the same manner, glycosyl transfer reaction between dTDP-mycarose and vicenistatin aglycon was also confirmed.

Mass spectral data: [M+H]⁺; calculated MW: 502.35; measured MW: 502.3

Moreover, glycosyl transfer reaction between dTDP-2-deoxyglucose and vicenistatin aglycon was also confirmed.

Mass spectral data: [M+H]⁺; calculated MW: 504.32; measured MW: 504.1

These results suggested that the recombinant VinC enzyme produced by the recombinant E. coli exhibited enzymatic activity as a glycosyltransferase. Therefore, it was confirmed that the VinC enzyme derived from the vicenistatin biosynthetic gene cluster plays a role as a glycosyltransferase.

This invention provides base sequence of the vin gene cluster, which is a cluster of genes encoding an enzyme complex catalyzing biosynthesis of vicenistatin. This invention further provides a transferase (VinC enzyme), which catalyzes synthesis of vicenistatin using dTDP-vicenisamine as a substrate, as well as a gene (vinC gene) encoding the enzyme. The VinC enzyme is useful for enzymatic synthesis of vicenistatin, which is a valuable compound. It is further suggested that use of the VinC enzyme may be useful for synthesis of novel polyketide glycosides.

What is claimed is:

1. A gene cluster comprising a base sequence represented by base Nos. 1-64492 shown in the SEQ ID No. 1 in the sequence listing.

2. A polypeptide comprising an amino acid sequence of the following (a) or (b):
(a) a polypeptide comprising an amino acid sequence represented by amino acid Nos. 1-419 shown in the SEQ ID No. 4 in the sequence listing,
(b) a polypeptide comprising an amino acid sequence having at least 90% homology with the amino acid sequence (a), and exhibiting enzymatic activity as a glycosyltransferase.

3. A gene encoding a polypeptide according to claim 2.

4. A gene comprising a base sequence the following (c), (d) or (e):
(c) a gene comprising a base sequence represented by base Nos. 2790-4049 shown in the SEQ ID No. 1 in the sequence listing,
(d) a gene comprising a base sequence having at least 90% homology with the base sequence (c), and encoding a polypeptide exhibiting enzymatic activity as a glycosyltransferase,
(e) a gene comprising a base sequence that hybridizes with the base sequence (c) under a stringent condition, and encoding a polypeptide exhibiting enzymatic activity as a glycosyltransferase.

5. A polypeptide comprising an amino acid sequence represented by amino acid Nos. 1-355 shown in the SEQ ID No. 2 in the sequence listing.

6. A polypeptide comprising an amino acid sequence represented by amino acid Nos. 1-323 shown in the SEQ ID No. 3 in the sequence listing.

7. A polypeptide comprising an amino acid sequence represented by amino acid Nos. 1-2260 shown in the SEQ ID No. 5 in the sequence listing.

8. A polypeptide comprising an amino acid sequence represented by amino acid Nos. 1-3362 shown in the SEQ ID No. 6 in the sequence listing.

9. A polypeptide comprising an amino acid sequence represented by amino acid Nos. 1-3808 shown in the SEQ ID No. 7 in the sequence listing.

10. A polypeptide comprising an amino acid sequence represented by amino acid Nos. 1-236 shown in the SEQ ID No. 8 in the sequence listing.

11. A polypeptide comprising an amino acid sequence represented by amino acid Nos. 1-397 shown in the SEQ ID No. 9 in the sequence listing.

12. A polypeptide comprising an amino acid sequence represented by amino acid Nos. 1-493 shown in the SEQ ID No. 10 in the sequence listing.

13. A polypeptide comprising an amino acid sequence represented by amino acid Nos. 1-330 shown in the SEQ ID No. 11 in the sequence listing.

14. A polypeptide comprising an amino acid sequence represented by amino acid Nos. 1-158 shown in the SEQ ID No. 12 in the sequence listing.

15. A polypeptide comprising an amino acid sequence represented by amino acid Nos. 1-469 shown in the SEQ ID No. 13 in the sequence listing.

16. A polypeptide comprising an amino acid sequence represented by amino acid Nos. 1-299 shown in the SEQ ID No. 14 in the sequence listing.

17. A polypeptide comprising an amino acid sequence represented by amino acid Nos. 1-327 shown in the SEQ ID No. 15 in the sequence listing.

18. A polypeptide comprising an amino acid sequence represented by amino acid Nos. 1-82 shown in the SEQ ID No. 16 in the sequence listing.

19. A polypeptide comprising an amino acid sequence represented by amino acid Nos. 1-524 shown in the SEQ ID No. 17 in the sequence listing.

20. A polypeptide comprising an amino acid sequence represented by amino acid Nos. 1-478 shown in the SEQ ID No. 18 in the sequence listing.

21. A polypeptide comprising an amino acid sequence represented by amino acid Nos. 1-414 shown in the SEQ ID No. 19 in the sequence listing.

22. A polypeptide comprising an amino acid sequence represented by amino acid Nos. 1-5836 shown in the SEQ ID No. 20 in the sequence listing.

23. A method for production of vicenistatin by catalytic reaction of the glycosyltransferase comprising the polypeptide according to claim 2, using dTDP-vicenisamine as a starting material.


SEQUENCE LISTING (26 sequences)

SEQ ID NO: 1 - DNA, 64492 bp, Streptomyces halstedii sp.HC-34
agatctggtgaatccgatgggcctcgggctgccggagcgtgcgggactccagctcgctctcgcccggcagtaatgcgtccactgtgacgatttcagcgatatgcgcggcttgatcggcgggcaggggcacgctccggaagagctgatccaccacgccgagttgtagctcacgctctgccttggccccggtcgccgcgagatgcagcgcgcccgagtcgagagcgtggtgtgacagtgcctccaggagggcagtcttgccggaggctatggcgccactgatcagtacggctctcccgtgcccccgggtggcgtcacccagcaaccctcccaaggcgtgcagttcctcgtcgcgttccacgaacaccatcgtcgcaaccctctccccgttcagatgtcgcgtacgtacgtatgaatatatcgcatgccacttgccgggtgaatataaattttactttacttacggattacttgcgctctgtcgtgtcgtcatggcaagggcgctcacccttcaacgtaggtgctacgggccgacataggggccgccccgggccgattaggggttatcccggccggggcccccgagtaaaaatgctgtcgccggaccgcggaaaaattcacggcggcgacgcgacccccaacggcgtgtccccgctcctgcgggtatctctccagccggctagacctagggggttgggtgaaagcgctcgttttgtctggcggatccggtacacggctccgaccgatcacacacacatcagccaaacaactggtgcccgttgcgaacaagcccatcctgttctacgtcctggagtcgatcgccgaagcgggcatcaccgacgttggcatcgttgtcggacacaccgctccggaggtccaggacgcggtcggtgacggctccgccttcggggtggacgtcacctacatcgcccaggacgagccgctcggcctcgcccacgcggtccgcatctcccgcgactacctcggtgatgacgactttgtcatgttcctcggtgacaacttcatcatcgacggcgtcaccggacttgtcgaccgcttccgtgatgagcgccccgacgcgcagatcctgctgacccgggtccccgacccccgggccttcggcgtcgccgtcctcgacgaacagggccgcgtcatcggcctggaggagaagccagaacacccccgcagcgatctggcgctcgccggcgtctacttcttcacccccctcatccacgaggccgtctgggccgtcaaaccctcctggcgcggcgaactggagatcactgactccatccagcacctcatcgacaccggagccgacgtccgctcccacatcatcgacgggtactggaaggacaccgggaacgtcgtcgacatcctggaggtcaaccgcatcatcctggagagcgtcgagaccgccatcgacggcgacgtcgacgcggactccgagatcatcggccgggtcgtcatcgagaagggcgccaccgtcaagggctcgcggatcgtgggccccgccctcatcgccaccggaaccgagatccgcgactcctacgtcggacccttcacctccgtcgccgagaactgtcgcatccacgacagcgaactggagttctccatcatgatgcgggactcgtccctcaccggcatccgccgcatcgaggcgtcgctcatcggccgccatgtccacggcaccgccgtaccccgcgtacccaacgcccaccggctcgtcctcggcgaccacagcaccttccagatcagctcatgagcacccgactgctcgtcacgggcgccgccggattcatcggatcggcctacgtccgcggactcctcaccgaccagcccgacctgcgggtcaccgtcctcgacagcctcacctacgcgggcaaccgggccaacctcgacctgacacacccctccctcgacctcgtcgagggtgacatctgcgacaccgagctcgtggaccgcctcaccgccgaagccgaccagatcgtccacttcgccgccgagagccacgtcgaccggtccatcaccggctccgccgagttcatccgcaccaacgtcctcggcacccacaccctcctcgacgccgcgctccgccacggcatcgaccggttcgtccacatctccacggacgaggtctacggatcgatcgagaagggctcctggccggagaccgacccgctgcgccccaactccccctactcggcgtccaaggcatccagcgacctcctggcgctcgcctaccaccggacccacgggctcgacgtacgggtcacccgttgctccaacaactatggcccctaccagcatcccgagaaggtcatcccgctcttcgtcaccaacctcctcgacggcaagcgggtacccctgtacggggacggccagaacgtccgcgactggctgcacgtcgaggaccactgcgccgccatcgaatgcgtccgcacccgcggtggcgccggggagatctacaacatcggcggcggcaccgaactgagcaaccgggaactcaccggcctgctcctggaggcgtgcggggcggactgggacagcgtggagtacgtcaccgaccgcaagggccacgacctgcgctactccgtcgactggtccaaggtcgccgacctcggctacacccccgcccacgacttccgtgccgggctcgcggagaccgtcgactggtaccgctccaaccgcacctggtgggaaccggcgaagtacggccacagccccgcctgaccggccctaccgccggccgacactcccacacccgcaccttcgctcggagaagaaaccccatgcgcgtcctgatgacggtgttcgccaaccgctcccacctctacaacatggtgcccctcgcctgggccctgaccaccgcgggccacgaggtccacatcgccagccaccccgacaacgtgcaggcgatcagcgacagcggcctcaccgccgtacccgtcggcaacgacctcaacatcatggcgctggcgcagtccaccccgcgcgaggagatggtcaacggcggcgcgctcaccctcaacgagacccggcccgagaagctgacctggcagtacatccacgacgtgttcgcgcagtactcccagatatacgagtacatggcggactcgaccatgaccgccgacctggtcgcccacgcccgccagtggcagcccgacctcgtcatctgggacgccctcacctacgccggccccatcgccgccgaagccgtcggcgcaccgcacgtccggatgctcttcggcctcgaccagtggggccggatgcgcgaccacttcaaccggctcaccggggaacgcgccgccgacgaccgccacgacccgctcgccgactggctcgcaaccaagggcgagccgcacggagtggccttcaccgaatccctcgtcaccggcacgaccaccctcgcggtcgccccgccgtggatgtcgttccccagcgagcagcccgccctctccatgcggcacctccccttcaacgggcccgccgtgctccccgactggctccgcgaggcccccagcaggccgcgcgtctgcctcaccctcgggctgaccctgcgtgaactcgccgacgacaacgtcaccctcgccgacttcgtcaacgccgtcgccgacatcgacgccgacgtcgtggcgaccttctccgcggaacaggtcgccgagatcggcgacctgcccgacaacgtccgcgcggtggacttcgtacccctacacgccctgctgccgagctgcgccgcgatcgtccaccacggcggcggcggcacccgcaccaacgccatccggtacggggtgccccagctgatcgtccccaactggctctgggacgaggggtatgtggccgagcgcttcgccgagcgtggcgcggcactcgtcaccgaggtccccgacctcacccccgaccggctgcgcgaccagctccgcaggctcatcgccgagccgtccttcaaggcggcggcggagcagatccagaaggagtacgacgcgctgcccagcctcaccgagaccgtcggtgagctggtgcgcgtcgcggagcgcgggcgctccctgtagcactccgcccagcggtacggggggccgctgggcggcccgccgtaccgagggcgtgacccgtatcgattcggccatccttggcgcggcagacccctgccggtccgctccgggcaaccccgagacggggatggtcctctaagggggcgtaggggttggcggcccccacccctgcccccgtagcgtgggaatcgttcaggctcgctggcgattgtccaatcaaatagttaagcccgtagattccatcgagccttttcgagaactgcgggttcgctttgtcgtgctctccgcacccgaatcatcgggtgcccagcagagttccgacagccgctcattcaaccgaacccgtttcacgaggtgatggcgtggagaacgaaaagaaacttctcgattacctcaagcgggctaccacggacctacgtgaagcacgccgccgcctccgcgagatggaggaaaaggaccaggagcccatcgccatcgtcggcatcggctgccgctttccccgcggtgtcgagtcggcggagcagctctgggacctcgtcgccgatggcggcgaggcgctcaccccgttccccgaggaccggggctgggacaccgacagcctgtaccaccccgacccggagcacctgggcaccagctacaccaacgtcggggccttcctgcacgacgccgccgagttcgaccccggcttcttcgggatctcgccgcgcgaggccctcgccatggacccgcagcagcgactgctgcttgagacgtcctgggaggccatggagcgggcgggcatcgacccggccggcctgcgcggcagccgcaccggtgtcttcacgggtctgatgtacttcgactacgggtcccgggtgcactccgcccccgaggacatcgagggctacctcggcaacggcagcgcgggcagcatcgcgtccggccgggtcgcctacaccttcggcttcgagggccccgccgtcacactcgacaccgcctgctcgtcgtccctggtcgccatccacctcgccgcgcagtccctgcgcaagggcgagtgcaccctggcgctggccggtggcgcgtcggtgatgtcgacgccggacatcttcgttgacttcagccgccagcggggtctgtccgccgacggccggtgcaaggcgttctcgtccgacgcggacggtacgggctggggcgagggcgtcggtgtcctgctgctggagcggttgtcggacgcgcggaagaacgggcaccggatcctgggcctggtacgcggctccgccgtcaaccaggacggcgccagcagcggtctgacggcgccgaacggtccttcgcagcagcgggtgatccgccaggcgctggccaacgccggtctgtcggccgccgaggtggacgcggtcgaggcgcacggtacgggtacgaagctgggtgacccgatcgaggcgcaggcgctgctggcgacctacggccaggagcgggaagagggccggccgctctggctcggttcgatcaagtcgaatgtgggtcacacgcaggctgccgcgggtgtcgccggtgtgatcaagatggtgctcgcgatgcgggccggtgtgctgccgaagacgctgcatgtgtcggagccgtcgccgcacgtggactggtccgccggcgcggtggaactgctgacggagacgcgcgactggccggagaccggccgtccgcgccgcgccggtgtgtcgtcgttcggtatcagtggcacgaacgcgcacgtcatcgtggagcaggcgcccatggacgaggcgatggacgaggtggccggcgagcgggagacccctgccgcggggtcggaccggaccgtcccgtgggtcatatcggcgaagtccgccgacgcgctccgcgcacaggccggacggctgagcaccttcctcgccgggaccgagggaactctgactactggtatttcttccgaaacttctgtggccgctgtttcagcggatgcgtccgcggttggttggtcgttggtgcggggtcgttcggtgttcgcgcatcgggcggttgtggtgggtggtgagtgggacgcgctgctggcgagtatcggtgaactggccgatggcgccgaggatggttccggtgctgcctctggttctgttgtgtcgggtgtggcggatgtgtcggggcgtcgggtgtttgtgttcccggggcagggttcgcagtgggttggtatggcgcaggggttgttggattcgtcggtgatgttcacggagcggatgacggagtgtgctgcggcgttggatccgttggtggagtggtcgttgttggatgtggtgcggggtgtggagggtgcggcgtcgttggagcgggtggatgtggtgcagccggtgttgtgggcggtgatggtgtcgttggcgtcggtgtggcgttcggtgggtgtggtgccggatgcggtggtgggtcattcgcagggtgagatcgcggctgctgtggtgggtggttggttgtcgttggtggatggtgcgcgggtggtggcgttgcggtcgttggcgattcgtgaggtgttggcgggtggtggtggcatggtcgcggtccaggctgcggaggatgaggttgctgggtggcttgagggtgtggagggggtggggattgctgcggtgaatggtccgcgttcggtggtgatctcgggtacgcgtgcgggtttggatgcgtgtgtggagttgtggtctgggcgggggacgtgggtgaagcgggttccggtggactatgcctcgcattcggctgaggtggagcgggtgcgtgagcgggtgctggcggatctggcgagtgtgacgggtttgtcggggtcggtgccgatgttgtcgacgatgacgggtgactggattgttgagggtcaggttggggccgggtattgggtggagaatctgcgtcgtccggtgttgttcgcggacgcgacgaggcggttggcgtcggagggtttcggcgcgttcgttgaggtgagtgcgcatccggtcctggtcatgggcatcgaggagacgatcgaggcgctgaggtcgggtgccgccgatggtgcgaagtccggtgctggtgaggagagttcctccgctgtggtggcggtggggacgttgcgtcgtggtgagggtggttgggatcagttcctgcgttccctcgcggggctcttcgtccggggtgcggtgacaccggattgggagtcgttgttgggtggtgtgcgtcctcgggttgatttgccgacgtatgcgttccagcgtgagcggttgtggctggatgcgggtgtggtggcgggggatgtgtcggggttgggtcaggtggtggtgggtcatccgttgttgggtgctggtgtgggtgttgctggtgagggtgggggtgtgttgtttacgggtcgtttggggttgggttcgcatccgtggctgggtgatcatgcggtgtcgggtgtggtgttgttgccgggggctgcgtttgtggagttggtggtgcgtgcgggtgatgaggtggggtgtggtcggttggaggagttgacgttggcggctccgttggtggtgccggagcgtggttcggtgcggattcaggtggtggtgggtgctggtgatgggtcgggtgcgcgttcggtgggtgtgtggtcgtcggtgggggatgagggtgtgggtggggagtgggtgtgtcatgcgtcgggtttgttgacggctgatgtgggtgtggcgccggtgttgggtggtgtgtggccgccggtgggtggtgtggcggtggatgtgtcgggtgtgtatgaggggttggcgttggaggggtatgagtatgggtcggtgtttcgggggttgaggtcggtgtggcgtcgtggggatgaggtttttgctgaggtggcgttgggtgagggtgtgggggtggaggggtttggtttgcatccggcgttgttggatgcggcgttgcaggctgctgggtttggttcgtttgtgccggagtccgaggcagggtctgaggcgggttcgggtggggtgcggttgccgttctcgtggtcgggtgtgtcgttgtttgcgtcgggtgcttcggtggggcgggtgcggttgtggccggtgggtggggatggttttggtgtggagttgtttgatggggtggggatgccggtggcgcgggtggatgcgttggtgacgcgggagattagtgcgggtcagttgggtgcggctgctggtgccgggtcgttggtgggtggggagtcgttgttccgggtggagtgggctcctgtgtcgggtgttgcaccggcttctgctggtgtgggtggttgtgtggtggtgggtgcggggagtgtgttgtctggttttggggaggtggttccggatctggcggcggtttccgcgggttctgcggctgtgccggggtgggtgttggtggatgtggatgcgtggttgggtgcggatctggcggtgggtgtggtgtcgggtgagggtgttccggtggtggcgcgtggtgtggtggcgcgggtgttggggttggtgcgggagtggttgggggatgagcggtgggtgtcgtcgcggttggtgtgggtgacgcgtggtgcggtgggtgctcgggtgttggatgaggtgtcgggtgtggtgtcgtcggggttgtgggggttggtgcgggctgctcagtcggagcatccggaccggttcgcgcttcttgatctggacagtgccaccgccgtggatgccgttcgtgatggtgtgttggggttgttggctgctggtgagccgcagttggtggtgcgtgagggtgaggtgctggccgcacggctgacccccgcccacaccaccgacgccctgatccccctcgccgaccacgccccgtggcgcctcgccaaggaccccggtggcagccttgacgcgctgacggtcgtcccggcccccgacgtactggagccgctcaccgagggacaggtccgcatcgcggtccgcgccgccggggtgaacttccgtgacgtactcatggcactcggcatggtccccgcccggggtacccagctcggtggcgaggccgccggcgtcgtgaccgctgtcggccctggagtcaccgggatcgccgtcggcgaccgcgtgatgggcgtcttcgacggcccgttcggcccggtggccgtcgcggatcggcgcatggtctcccgtatccccgatgcctggtcgtacaccgaggccgcgacgatcccgctggtgtacctcaccgcgtactacggactggtggacctcgccgctctccagcagggacagcgcatcctggtgcacgccgccaccggtggcgtgggcatggcagcggtccagctcgcccgccacttcggcgccgaggtgtacggcacggccagccccggcaagtgggacaccctgcgcgcgatgggattcgacgaggcgcatatggcctcctcgcggtccctcgacttcgaggaccacttctggcagacgacgggtggcgaaggcttcgacgtcgtactcaactccctcgcgcaggagtacgtggatgcttcactgcggctccagccgcgcggcggccgattcctggagatgggtaagaccgatatccgggacgccgatgaggtcgcggcggcacacgagggagtgcgctatgaggcgtacgacctcaccgtcttcaccagcgtcgacggaccgggcgcgatccccgagcgcattcaggagatgctctccgaactgcttgcgctgttcgacaagggagtcctcaccccgctgcccgtcaccacctgggacgtccgccgcgcgtccgccgcgctgcggcacatgtcgcaggcccggcacaccggcaagatcgcgctgacggtgccgcgcccgctcgaccaggacggcacggtcctcgtcaccggtggcaccggtgtcctgggcagtctcctggcccgtcatctggtgaccgagcacggggtacggaatctgctgctggtgagccgtcggggcggcgacgcccccggcgccgccgaactggtcgccgagctgacggcagccggtgccgaggtgagcgtcgtcgcctgtgacaccgccgaccgggcagcgttggagaagctcctcgcgtccgtgccgggtgacgccccgctgacgggcgtcttccacacggcgggcgtgctggacgacggaatcgtcgaggcgatgaccccggagcgggtggacgccgtgatgcgccccaaggtggacgcggcctggcatctgcacgagctgaccgagggacttgatctcgcggccttcgtgctctactcctccgcagccggtgtctccggtgacgcgggccagtccaactacgccgccgccaatgtcttcctcgacgccctcgcacagcggcggcgggctgccgggctgcccggccagtccctcgcctggggcctgtgggacgaccggagcgagatgaccggtcacctcggcgacgccgagatcgcccggatgaccgaggccggtgtcctcggcttcaccgccgccgacggcctggccgcgctggaccgcgcggcgatgtacgacgacgccgtgctggtgccgatgaagctggacacggcgaccctgggcgcgggctcgtcgcccgtaccgcatctcttccgcggtctggtgcggacccccgtcgtacgccgggccgtcgccgggaacaccggcggggacagcggcggcggacttgagcagcggctcgccgctctcaccgcggccgagcgcaccgagaccgtgctggaactcgtacgggagcgcgtcgccgcggtcctcggacatgccagcgccgacgccatcgatcccgcccgggcgttcaaggagatcgggttcgactcgctcacggctgtcgaactgcggaacaggctgaacgcggccaccggactgcggctgcccgccacactggtcttcgactatccgacgcccacggtgctctcgcagtacctcctggcggagctggcacccggcttgccggccgaggacccggtcgggacccggctgctggagcagatcgcccgtatcgaagcggtcctctccgaggtctcggaggtcacggacgagacctcctccctctccgacatggacgccgatgcccgatcgggcatcaccgcacgcctcaacgacatcctgaccgcatggaacagggcccagcgcgcgccgggtcacgacgcggtcgcggcggaactggacgacgccagcgacgacgaaatcttcgatttcatcgacagcaccttcggcaagtcctgactcatccccgctgacccaccgacgccaaccccgcgtttccgcatacctggacgggtgaaccactcatggccaacgaagccaagctccgcgaatacctcaagcgcgtcaccaccgatctgcacgagaccaatgagcgcttgcgcgaggtcgagggcagggcgaacgaaccgatcgcgatcgtgggtatgagctgccgcttccccgggggtgtcgagtccccggagcagttgtgggagctgttccgtacgggtacggacgccatcggtgagttccccgaaggacgcggctgggacgtcgaggggctctaccacccggaccccgaccacgcgggcaccagctacacgcgtgaaggcggcttcgtccacggcgccgagcgcttcgacccgagcctcttcggcatctcgccgcgcgaggccgtgtcgatggacccgcagcagcggctgctccttgagacctcgtgggaggccctggaggcgagcggactcgacccgctccgcctcaagggcagccgcaccggtgtcttcgtcggtgtcatgtcctcggactacggcattcagaagggctcggcgcccgacggtgtcgagggcttcctgagcaccggtacgcactccagcatcgtctccggccgggtctcgtacgtcctgggtctggagggtccggcggtctcggtcgacaccgcttgctcgtcctcgctcgtcgccctgcactccgccgcgcacgccctgcgccagggtgagtgctccctcgccctggcgggcggcgtgacgatcatgtcgacgccggaacgtttcgtcgagttcagccgacagcgggccctgtccgccgacggccgctgcaaggcgttctcggcgagcgccgacggcaccggctggtccgagggtgtcggcatgttggtcctggagcggctctccgacgcgcggaagaacgggcaccgggtgcttgcggtcatccgtggttcggcgttgaaccaggatggtgcgagcaatggtctgacggcgccgaacggtccgtcccagcagcgggtgattcgtcaggcgctggcgagtgccggtctgacggcggccgaggtggacgctgtcgaggcgcatggcaccggcaccacgctcggtgaccccattgaggcgcaggcactgctggcgacctacggcaaggagcgcgaggacggtcgtccgctgctgctgggttcctccaagtcgaacctcggccacacccaggccgcggccggtgttgccggtgtgatcaagatggtgctcgcgatgcgggccggtgtgctgccgaagacgctgcatgtgtcggagccgtcgccgcacgtggactggtccgccggcgcggtggagctactcacggaggcccgggagtggccggagaccggccgtccgcgccgcgccggtgtgtcgtcgttcgggttcagtggcacgaacgcgcacgtgatcatcgaggaggcgtcggagttcgagccgagcgccgtggagccgctggccgggtcgggcgtgactccgccgtgggtgctgtcggcgcggtcggccgacgcgttgcgggggcaggcggagcggctgctgtcgttcgtctctgccgccggtgatgtgtcggtggtggatgtggcgtactcgctgggtgtgtcgcgtgcgggtcttgagcatcgtggtgtggtggtgggggagtcgcgtgcggagttgctcgcggctctggagtcgttggcgtccggggttgagtcgccgggtgtggtgacgggtcgggtcgctgagggtcggttggcgttcttgttcacggggcagggtgcgcagcgggttggtatggggcgtgagctggctgctgcgttcccgttgtttgcggcgtctcttgaggagacgtgtggcttgttggagcgtgccggggttgcggtgcgtgaggtgctgttcgccgaggagggttcggccgaggccgccctgctgacgcggacggtgtatgcgcaggcggcgttgtttgcggtggaggtggcgctgttccggttggtggagtcgttcggtgtggtgccggactttgtcgctgggcattcggtgggtgagatcgctgccgcgcatgtcgcgggggtgttctcgcttgaggatgcggtgtctctggtggcggctcgtggtcggttgatggatgcgctgccggagggtggggcgatggtggcggtgcaggccaccgaggaggatgtgctcgctctgttggagggggtggaggatgcctccatcgcggcgatcaatggccccgacgccgtggtggtctccggcaccgaggccggtgtcgcccgcgtagtggatgtactccgggagcggggtgcgaagaccaagcggctcgtggtgagtcatgccttccactcgccgctgatggagccgatgttggctgagttcgccaccgtcgtggaggggttgtcgttcgcggctcccaccattccggtggtgtcgaatgtgtcgggtgcggtggcggatgccgagttgtcgtcgccgggttactgggtgcggcatgtgcgtgaggcggttcgtttcggtgccggggtggagaccctgctcggcgctggtgtgtcgtcgttcctggagatcggtccggacggtgtgctgagcgggatggcccgcacgtccgtgcctgagggtgcggatgtggagtgcgcgccgctgatgcgccggggccgtggtgaggtgcgggagttcctcaccggcctctcccggatggtcgtacggggagtcccggtcgactggcagtcgctggtcgagggcggtcgcctggccggactgccgacgtacgccttccagcgtgagcggttctggctggacgttccttccgctgtcggtgatgtggccaccgccggacttgcgccctcgggccaccccctgctcggtgcggtcatgcgacgcgccgatgtcgatggtgtcgtgttcacgggccgctggtcactgcgcagtcatccgtggctgggtgagcaccgggtcgggtcttcggtggtgttcccggggaccgggttcgtcgagctgctgatgcgcgcgggcgacgaggtcggttgcggccggatcgaggagttgaaccaggagactccgctcgtcgtccccgagcgcggagcgctccagctccaggtggtcgtgggcgcgcccgaggagaccggcctccgtggtgtcggcgtgtactcgcgcgcggaggacgccgatgcggatgtgccgtggacccggcacgccagtggtctgctgagcccggccgtggtgccggccgacttcgagctgacgcagtggccgccggccggtgcggaggccctcaatgtcgaggacacgtaccagcgactcgctgatgccggactggtctacggcgagcggttccagggcctcaagtcgggctggatcaagggtgaggacatctacgccgagatcgcgctgcccgagcacgccgtcgccgaggcggccgagtacacgctgcacccggcggcgctggacgctgcccttcaggcgagcggtctgaacgatccgcccgaggtgcaggcgacggcctatgtccccttctcgtggtcgggggtgtcgttgtttgcttcgggtgcttcggtgttgcgggtgtgtgtgcgtcatgtggcgcgggatcgggtgtcgttgttggtggctgatggtgtgggtgtgccggttgcggtggtggagtcgttggtgttgcgggcgatttctgctggtgcggtggcggttgctggggtgggttcgggtgtgggtggtgggttgtttgaggtggtgtggtctccggttgttggtgtgaggggtgtggatgtgtcgggtgtggtggtgttggaggctggtgtgggtgttggtggggatggggtttcggtggtgggtggtgtgttggagggtttgcagggtgtgttgggtgggggttctggttctcgtgtggtggtggtgacgcggggtgcggttggttctggtggtgtggtggatgtgtcgggtgcgggtgtgtgggggttggtgcggtcggttcaggctgagcatccgggtcggttggtgttggtggatgtgggtgttgagggtgatgtgggtgtgggtgtggggttggctttgggttcgggtgaggagcaggtggtggtgcgtgggggtgaggtgtttgttcctcgtttggcgcgggtgggtgctgtggcggcggatgcgggtgttgatggtgctgagatcgcgggtggtttgggtgatggtgtggtgttggtgacgggtggtacgggtgggttgggtgcgttggtggcgcggcatgtggtggtggagcgtggggtgcgtcgtttggtgttggtgtcgcgtcgtgggttgggtgctccgggtgcggtggggttggtggctgagttggagggtttgggtgctgtggtggaggtggtggcgtgtgatgtgagtgatcgtgtggcgttggcgggtgtggtggggggtattgggtcggatctttcggctgtggtgcatacggcgggtgtggtggatgacggtgtggtggagtcgatgtcggtgggtcgggtggcgtcggtgtttggtccgaaggcggatgctgcgtggtttttgcatgagttgacgcgtgacatggggctgtcggcgtttgtgttgttctcgtcgatggcggggacggtgggtggtggtggtcagagcaactatgcggcggcgaatgcttatttggatggtttggcggagtatcggcgtgggttggggctggcggcgacatctctagcctggggactctgggaagagtcgacgggcatgggcagccgcctcaccgacgccgacctggacaggatgagccgctcgggcatccgtacgctctcgatcgaggacgggctggcgctcttcgacgcggccctcgccgccgaccggccgacagtcatgccggcccacttcgacatccccgccctgcggggacagggcgaatccctcgcgccggtcttccgcaccctggccggccccccggcccgccgctcggcggccgtcaccccccgtgacatcgcggcggcgaccgagagcagcctcaccgaccggctcgccggtctggacgcggaaggacgccgggcgctggtgctcggtgtggttcgtgcgcaggtcgcgcaggtgctggcctacgcctcaccggatctcgtggagccggagcgcgccttccaggacctcggcttcgactcgctgaccgccgtcgaactgcgcaacggcctcacggccatcgcgggagtccgcctcccggcgacgctcgtcttcgactacccctcgaccgacatcctcaccgacttcctcctcgccgaactctccgacgagattccggcggcggtggcgaccctgccggcgatgggccatgtcgtcgacgacgacccgatcgccgtcatcggcatgggctgccgctaccccggcggggtcgagtcccccgaggaactgtggaagctcatggcggagggccgggacgccatctccgagttcccgaccgaccgcgggtgggacctcgatgccatctaccaccccgacccgatgcacacgggcaccagctacacccgcgagggtggcttcatccacaacgcgggcgacttcgacgccgccttcttcgggatctcgccgcgcgaggcgatggagaccgacccccagcagcgactgctccttgagacgtcctgggaggcgttcgagcaggccgggatcgtccccaccgatctcaagggcactcagaccggtgtcttcgccggtgtcatgtaccacgactacgcgggcaacatcggctccggcagcatcgtcacgggccgcgtcgcgtacaccctgggcctggagggtccggcagtctccatcgacaccgcctgctcgtcgtccctggtcgccatccacctcgccgcgcagtccctgcgtcagggcgagtgctccatggccatcgccggaggtgtcgccgtcatggcgacgccggagtcgttcatcgaattcagccgccagcgcgcgctctcccagaacggccgctgccgcgccttctcctcggacgcggacggtacggcctggggcgagggtgtcggtgtcctgatcctggagcggttgtcggacgcgcggaagaacgggcacgaggtactcgcggtcatccgtggttcggcgttgaaccaggatggtgcgagcaatggtctgacggcgccgaacggtccgtcccagcagcgggtgatccgccaggcgctggcgaactcgggtctgtcggccgccgaggtggacgcggtcgaggcgcacggcaccggtaccacgctcggtgacccgatcgaggcgcaggcactgctggcgacctacggcaaggagcgggacgcggaccagcccctctggctcggttcgtccaagtcgaacttcggtcacaccaaggccgccgccggtgtcgccggtgtgatcaagatggtgatggccatcaggaacggtgtgctgccgaagacgctgcatgtcaccgagccgtcgccgcacgtggattggtctgctggtgcggtggagctgttggcggaggctcgggagtggcctgagacgggtcgtccgcgccgcgccggtgtgtcgtccttcggtatcagtgggacgaacgcgcacgtgatcgtggagcaggcccccaccgaccaggccgctaccaagccgaaggctgccgacgcggtcccgggcctgcccgttccgtgggtggtgtctgcgaagaacccggaggcgttgcgggcgcaggcgggtcggttggggtcgttcttgggtgagaccggtgttgttgatgtgccggcggtgggttggtcgttggtgcggggtcgttcggtgttcgcgcatcgggcggttgtggtgggtggtgagtgggacgcgctgctggcgagtatcggtgaactggccgatggcgccgaggatggttccggtgctgcctctggttctgttgtgtcgggtgtggcggatgtgtcggggcgtcgggtgtttgtgttcccggggcagggttcgcagtgggttggtatggcgcaggggttgttggattcgtcggtggtgttcacggagcggatgacggagtgtgctgcggcgttggatccgttggtggagtggtcgttgttggatgtggtgcggggtgtggagggtgcggcgtcgttggagcgggtggatgtggtgcagccggtgttgtgggcggtgatggtgtcgttggcgtcggtgtggcgttcggtgggtgtggtgccggatgcggtggtgggtcattcgcagggtgagatcgcggctgctgtggtgggtggttggttgtcgttggtggatggtgcgcgggtggtggcgttgcggtcgttggcgattcgtgaggtgttggcgggtggtggtggcatggtcgcggtccaggctgcggaggatgaggttgctgggtggcttgagggtgtggagggggtggggattgctgcggtgaatggtccgcgttcggtggtgatctcgggtacgcgtgcgggtttggatgcgtgtgtggagttgtggtctgggcgggggacgtgggtgaagcgggttccggtggactatgcctcgcattcggctgaggtggagcgggtgcgtgagcgggtgctggcggatctggcgagtgtgacgggtttgtcggggtcggtgccgatgttgtcgacgatgacgggtgactggattgttgagggtcaggttggggccgggtattgggtggagaatctgcgtcgtccggtgttgttcgcggacgcgacgaggcggttggcgtcggagggtttcggcgcgttcgttgaggtgagtgcgcatccggtcctggtcatgggcatcgaggagaccttggaggcccatcacaccgccacgaccgacgacgacaccaccacccgaacccccgtggtcaccgtcggcacgttgcgtcgtggtgagggtggttgggatcagttcctgcgctccctcgcggggctcttcgtccggggtgcggtgacaccggattgggagtcgttgctgggcggtacgcgtccccgggtcgacctcccgacctacgccttccagcgtcggcgcttctggatcgagagcgtcaggaaggaagcggtgacactcgccgccgacccggtcgacgccgcgttctgggaggccgtcgagagcgccgacctggcgaagctcgccgacagcctgcgcatcgagaccgacgtactcgaaggcgtactccccgcgctcacctcctggcggacacgcagccgtgagcagtcgctcgtcgacggctggcgctaccgcgaggagtggaagccggtgcccgcaccgaccccgaacagcgccaccggcacctggaccgtgctggtcccggccacccaccagggcgacgccaccgtgaccggtgtgctggacggcctgcgccgtggcggcgccgacatccgggtgctcgaggtcaccgccaccgaccgcgaggccctggccgagcagctccgcgccgaactcgcccagagcgagccccggctcatcctctccctgctggcactggacgaccgcgcccacccccgtcaccccgagctgaccgagggcatcaccgccaccatcgctctcgtccaggcgctggacgactgcggcgccaccgcccggctctggtgcgccacctcgctggccgtcgcggtcaccgaatcggccgaggtgctcaaccccgtacagacgaccgcctggggcatgggcgcgtccttcgcgctcgaccaccccgagacctggggcggactggtcgacctcccggccgacatcgacacccgtaccgcggacatcctctgctcggtcctggcgtcggacctccaggaagaccagatcgccctgcgtacggccggcctcttcgcccgccgtatggtccgggcccgcctcgacgaatccgccgtggcgacagagcagccgtggcggcccaacggcaccgtcctggtcaccggaggcaccggcgggatcggctcgcacgtggcgcgctggctggcggccgccggtgcggagcacctcgtactcaccagccgtcgcggtgccgaggcgcccggcgccgccgaactcgaagccgagctggccctgctgggtgccaaggtgaccctcgccgcctgtgacatgggagaccgcgagtccgtacgggaactcgtcgccgccctccccgacgccgcaccgctcacggccgtcttccacctggccggcgcactcccggacggcgagcgccgactctcggcgacgacgttcgaggacttctcccggatgacccgggcgaagatcggtggagccgttcacctggacgagctgctcgcggaccgcgagctcgcggccttcgtgaccttctcctccggctcggcgatctggggcaacgccaaccaggcggcgtacggcgcgtccaacgcgttcctcgacggcctcgtccacaaccggcgggcacgtgggctcgccgggacgtcgatcgcgtgggggctctggggcggtgacggcacggacaccgagaccgacgagcagctcagccgcatcggtgtcaggtcgatggaccctcggctggcccttgaggtcctccggcaggccctcgaccaggacaccagccatctgatcgccaccgacatcgactggcagcgcttcgccccggtgttcaccatcgcccggccgcgcccgctgctggacggaatcgccgaggtcagggccgtcctgtcggcggacaccgccgaggtcgggccggccgacgatgacaccccgaaggagaccgtcgtcacccggctcgccggcctcagccccgccgagcgtgaccacgccctgctggagctcgtacggacacaggtggccgcggtgctccgctactcggacacctcggacgtcgagcaggaccagtccttcaaggacctcggcttcgactccgtcaccgcggtcgaactgcggaacaagctcacacgcggcgtccgcgctgcggctgcccgccacggcgtcttcgactacgccaccccggtcgccctcgcggcccatctgcggtcggagctgttcacggacgacgccgcggcgtcgggagaggtgccgctgctggcggagctcgaccggatcgaggcggcggtgacctcgctcccgtcggccgacatcgagcggatgcacctcacctccaggcttcagagcctggtgaccaagttgaacgacatcgtcggcgcgggagctcccctggaagcggaggccatcgcggacaagctcgaaacggccactgccgatgacatcttcgcgttcatcgacaaggacctcggtctgaactgaccggcacgtccccgcctccccgcgctcccgctccctcgcacaaagattggtgtgtcaccggaatgtcgaacgaagagaagctcctcgactacctcaagcgggtcaccgccgatctccacgcgacccggcagcggctgcgtgaggcggagtccgatgagcaggagccgatcgcggtcgtcgcgatgggctgccgctacccgggggacgtccgtacccccgaggacctgtggcagctcgtcgcgacgggcggtgacgccgtcaccgaattccccgccgaccggggctgggacttcgacacactcctcggcggcgatgcgggcgcatccgggagcacctatgtggcccgcggcgggttcgtccacgacgcggccgacttcgatgcggacttcttcggcatctcgccgcgcgaggcgctggcgatggacccgcagcagcggctgctgctggaactcgcctgggagaccagcgagcgggccggcatcgacccgcacagcctgcggggtgcctccgtcggcgtcttcgccggcaccaacggtcaggactacgccgacctgatggaccgggcgccggaggacaccgaggcatacctctccaccggtagcgtcgcggccgtcgtctccggccgggtctcgtacgcgctgggtctggagggcccttcggtctcggtcgacaccgcctgctcctcctcgctcgtcgcgctgcacctcgcctgccaggcactccggcagaaggagtgctccatggccttcgcgggtggcgtcacgatcatgtcgacgcccgggccgttcatcgccttcagcaggcagagcggtctcgcgtcggacggccggtgcaaggcgttctccgacgacacggacggtacggggtggggtgagggcgcgggcctcctgctgctggagcggctctcggacgcgcgcaggaacgggcatcaggtgctcgccgtggtgcggggctcggcgttgaaccaggatggtgcgagcaatggtctgacggcgccgaacggtccgtcccagcagcgggtgatccgccaggcgctggccaacgccggtctgacggcggccgaggtggacgcggtcgaggcgcacggcaccggtacgaccctcggtgacccgatcgaggcgcaggccatcctcgccacgtatgggcagagccgcgagcaggaccagccgctctggctcggctccatcaagtcgaacatcggtcatacccaggccgcggccggggtcagcggtgtgatcaagatggtgatggccatccagaacggcgtgctgccgaagacgctgcatgtgtcggagccgtcgtccgttgtggattggtccgctggtgcggtggagctactcacggaggctcgggagtggccggagaccggtcgtccgcgccgtgccggtgtgtcgtcgttcggtgtgagcggtacgaacgcccacatcatcatggagcaggcgcccgtgcctgaggcagagtcggagccggacggcgaagcacccgccgcggtgtccgggctgccggttccttgggtggtctccgggaagaccgccgacgcgttgcgggcgcaggccgagcggctgctgtcgttcgtctccgccgacgcggatgtctccgtggtggatgtggcgtactcgctgggtgtgtcgcgtgcgggtcttgagcatcgtggtgtggtggtgggggagtcgcgtgcggagttgctcgcggctctggagtcgttggcgtccggggttgagtcgccgggtgtggtgacgggtcgggtcgccgagggtcggttggcgttcttgttcacggggcagggtgcgcagcgggttggtatggggcgtgagctggctgctgcgttcccggtgtttgcggcgtctcttgaggagacgtgtggcttgttggagcgtgccggggttgcggtgcgtgaggtgctgttcgccgaggagggttcggccgaggccgccctgctgacgcggacggtgtatgcgcaggcggcgttgtttgcggtggaggtggcgctgttccggttggtggagtcgttcggtgtggtgccggactttgtcgctgggcattcggtgggtgagatcgctgccgcgcatgtcgcgggggtgttctcgcttgaggatgcggtgtctctggtggcggctcgtggtcggttgatggatgcgctgccggagggtggggcgatggtggcggtgcaggccaccgaggaggatgtgctcgctctgttggagggggtggaggatgcctccatcgcggcgatcaatggccccgacgccgtggtggtctccggcaccgaggccggtgtcgcccgcgtagtggacgtactccgggagcggggtgcgaagaccaagcggctcgacgtcagccacgccttccactcgccgctgatggagccgatgttggctgagttcgccaccgtcgtggaggggttgtcgttcgcggctcccaccattccggtggtgtcgaatgtgtcgggtgcggtggcggatgccgagttgtcgtcgccgggttactgggtgcggcatgtgcgtgaggcggttcgtttcggtgccggggtggagaccctgctcggcgctggtgtgtcgtcgttcctggagatcggtccggacggtgtgctgagcgggatggcccgtgggtcgatctccgacggcgccgacgttggctgtgtccccgtgatgcgccggggccggggtgaggtgcgggagttcctcaccggcctctcccggatcgccgtacggggagtccccgtgagctggggtccgctgctggcgggtggtcggcgggtcgagctgccgacgtacgccttccagcgtcgccggttctggctggaggcggggcactccgtcacggacgcttcgggtctgggccagacggctgccgggcatccgctgatcggtgctgtggtgagtctggccggtggtgacggtgccgtgctgaccggtcgggtgtcgttgaacacgcatccctggctgggcgaccaccgggtcgcgtcttcggtggtgttcccggggaccgggttcgtcgagctgctgatgcgcgcgggcgacgaggtcggctgcggccggctggaggagctgaaccaggaggcaccgctcgtcgtacccgaccggggcgccgtccagatccaggtgaccgtggaggcccccaatgctgtgggagagcgaccggtcgcggtttactcgcggctggaggacacggatacggacgccccgtggacccggcacgccagtggtctgctgagcccaaccaccgcttcggcggacttcgacttcacggcatggccgccggccggtgcggaccccctctccgtcgacggaatgtatgaccgcccggactcgggcctggtctacggtcccctgttccaggggctgacggccgcctggcgcaagggcgatgaggtgtacgcggagatcgagctgcccgagggggcggcggtcgacgccgcccgcttcgggatgcaccccgccctcctcgacgccgccctccacgccctcgggttctcggcctcctacgaggaccaggaggaggacggcaccgtggcccggctgcccttctcgtggtcgggggtgtcgttgtttgcttcgggtgcttcggtgttgcgggtgtgtgtgcgtcatgtggcgcgggatcgggtgtcgttgttggtggctgatggtgtgggtgtgccggttgcggtggtggagtcgttggtgttgcgggcgatttctgctggtgcggtggcggttgctggggtgggttcgggtgtgggtggtgggttgtttgaggtggtgtggtctccggttgttggtgtgaggggtgtggatgtgtcgggtgtggtggtgttggaggctggtgtgggtgttggtggggatggggtttcggtggtgggtggtgtgttggagggtttgcagggtgtgttgggtgggggttctggttctcgtgtggtggtggtgacgcggggtgcggttggttctggtggtgtggtggatgtgtcgggtgcgggtgtgtgggggttggtgcggtcggttcaggccgagcatccgggtcggttggtgttggtggatgtgggtgttgagggtgatgtgggtgtgggtgtggggttggctttgggttcgggtgaggagcaggtggtggtgcgtgggggtgaggtgtttgttcctcgtttggcgcgggtgagtgttgtggcggcggatgtggtgccggatgcgggtgttgatggtgccgatatcaggggtggtttgggtgatggtgtggtgttggtgacgggtggtacgggtgggttgggtgcgttggtggcgcggcatgtggtggtggagcgtggggtgcgtcgtttggtgttggtgtcgcgtcgtgggttgggtgctccgggtgcggtggggttggtggctgagcttgagagcttgggtgctgtggtggaggtggtggcgtgtgatgtgagtgatcgtgtggcgttggcgggtgtggtggggggtattgggtcggatctttcggctgtggtgcatacggcgggtgtggtggatgacggtgtggtggagtcgatgtcggtgggtcgggtggcgtcggtgtttggtccgaaggcggatgctgcgtggtttttgcatgagttgacgcgtgacatggggctgtcggcgtttgtgttgttctcgtcgatggcggggacggtgggtggtggtggtcagagcaactatgcggcggcgaatgcttatttggatggtttggcggagtatcggcgtgggttggggctggcggcgacatccctggcctggggactctgggaagacaccgcgaacggcggtatgacgggccacctggccgaagccgaccgtacgcggatggcacgcgggggagtcttccctctggcgctggacgaggggctcgcactcttcgacgccgccctcgccaccggaaaggcgacactcgtccccgtccatctggacaccaccgcgctgcgcgcacacgctgacgaactgcccgccctcttccgtgacctcgtccgcgcgccgaagcggcgcacggcggcggaaggccgtgcggcggactccgccgacgacctcgcgggccgcctcgccggcctggccgccgaggcgcgccggccgttggtgctcggtgtggttcgtgcgcaggtcgcgcaggtgctggggtacgcctcggcggatctcgtggagccggagcgcgcgttccaggacctcggcttcgattcgctgacggccgtcgaactgcgcaacggcctcacggccgtcgcgggggtccgcctcccggcgacgctcgtcttcgactatccctcgaccgacatcctcaccgacttcctcctcgccgaactctccggcaaggtcgccgtggccgcgcccctcgcgccgctcgccaccaccgggccggtgcacgacgacccgatcgtcgtcatcggcatgggctgccgctacccaggcggggtccgctcgccggaggacctgtggcggctggtcgccgatggccgggatgcgatatcggagttcccgaccgaccgcggctgggacctcgacgcgctgtaccacccggacccggaccacgccgggaccagctacacccgcgagggcgggttcctgcacgacgcggccgatttcgacgcggacttcttcgggatctcgccgcgtgaggccatcgtcatggacccgcagcagcggctgctccttgagacgtcctgggaggcgttcgagcaggccggcatcgtgcccgccgacctcaagggcacccagaccggcgtcttcgccggagtgatgtaccacgactacggcacccgaatcgtcgacattcccgagggggccgaaggctatctgggcaccggcatctccggcagcgtcgtctccggccgggtcgcgtacaccctgggtctggagggcccggcggtcacgatcgacaccgcctgctcctcctcgctcgtcgccctgcattccgccgcgcacgccctgcgccagggtgagtgctccatggccatcgccggtggtgtcaccgtcatggcgggccccgacaccttcatcgacttcagcaggcagcgcggtctggccacgaacggtcgctgcaaggcgttctcggcggacgccgacggcaccggctggggcgagggcgtcggcgtcctggtcctggagcggttgtcggacgcgcggaagaacgggcacgaggtactcgcggtcatccgtggttcggcgctgaaccaggacggtgcgagtaacggtctgacggcgccgaacggtccttcgcagcagcgggtgatccgccaggcactggccagcgccggtctgacgacgtcggacgtcgacgcggtcgaggcgcacggcaccggcaccacgctcggtgacccgatcgaggcccaggcggtgctggccacctacggccaggatcgcgccgcggaccagccgctctggctcggctcggccaagtcgaacttcggtcacacgcaggccgcggccggtgtcgccggtgtgatcaagatggtgatggccatccggaacggcgtgctgccgaagacgctgcatgtgtcggagccttcgacacatgtggactggtccgccggcgccgtggagctgctggcggaggcccgggagtggccggagaccggtcgtccgcgccgcgccggtgtgtcgtcgttcggtgtgagcggtacgaacgcgcacgtcatcgtcgagcaggcccccaccgaggagcaggcccccgcggacgcgcctgccccgacggacgcgcccgccggcactccggtgccctggatcgtctccggtcggaccgccgactcactgcgcgaccaggcccgccgtctgctggagcacctcgaccggaacggcgacctcgacccgcaggacgtcgcgcgggccctcctcaccacccgtacccgcttccaccaccgtgccgccgttgtggcgaccgagcgacaggacatcgtcgcggcgctcgaagcactcgccgacggccggcccgtgagcggtctggttcaggggacggccaccaccatggcgaagtccgcgttcctgttcacggggcagggtgcgcagcgggtcggcatgggtcgggaactcgcggccgaattcccggtgtttgcggcgtctcttgagcagacgtgtggtctgttggagcgtgccggggttgcggtgcgtgaggtgttgttcgccgaggagggttcggccgaggccgccctgctgacgcggacggtgtatgcgcaggctgcgttgttcgccgtcgaggtggcgctgttccggttggtggagtcgttcggtgtggtgccggactttgtcgctgggcattcggtgggtgagatcgctgccgcgcatgtcgcgggggtgttctcgcttgaggatgcggtgtccctggtggcggctcgtggtcggttgatggatgcgctgccggagggtggggcgatggtggcggtgcaggccaccgaggaggatgtgcttgccctcctggagggggtggaggacgcctccatcgccgcgatcaacggccccgacgccgtggtcgtctccggcaccgagaccggtgtcgcccgcgtagtggacgtactccgggagcggggcgcgaagaccaagcggctcgacgtcagccacgccttccactcaccgctgatggagccgatgctcgccgacttcgcacgggtggtcgcgggtctgtcctatgaggaccctgcgatcccggtcgtgtcgaatgtgtcggggtcgatggcggacggtgagctgtcgacgccgggatactgggtgcggcatgttcgtgaggcggttcgtttcggtgccggggtggagaccctgctcggcgctggtgtgtcgtcgttcctggagatcggccccgatggtgtgctgagcgggatggcccgtacgtccgtacctgagggtgcggatgtggagtgcgcgccgctgatgcgccgagaccgcgccgaggtacgggagttcctcaccggcctctcccgtctcgccgtacgcggtgtcccggtgagctggtcccccctcgtggcggggggccggcgggtcgagctgccgacgtacgccttccagcgtcgccggttctggctcgacgccggacactcggtcacggacgcttcgggtctgggccagacggccgccgggcatccgctgatcggtgccgtggtgggtctggccggtggcgatggtgtcgtcctgaccggtcgggtgtcgttgcacacccacccctggttggcggatcaccaggtcgcgggggtgacgctgcttccgggtacgggctttgtggagctggcggtccgtgccggtgacgaggtcggctgtggccggctggaggaactgaccctggaggccccgctggtcgtgcccgaccggggcgccgtccagctccaggtcgtcgtcggcgggctggaggaatccggcgtccgtaccgtcagcgtctactcccgagccgaggacaccgacaaccccgacaccccctggacccggcacgccagtggtgccctcggcacggcggccgacccggccgacttcgacctcaccgcctggccgccggccggaaccgaggccgtcgagatcggggacttctacggcgaactggccgccacacccgacggtctggtctacggtccgctgttccaggggctgacggccgcctggcgcaagggcgatgaggtgtacgcggagatcgagctgcccgagggcgcggcggtcgacgccgcccgcttcgggatgcaccccgccctcctcgacgccgccctccacgccgtcggactctcgaacgacgccgacgccgacgccgacgcgggcacggcacaggaggacggcaccgtggcccggctccccttcgcctggtccggtgtgacgctccaccgctccggcgcgacccggctgcggctctccgtacggcccaccggaggcgacagctaccacctgcggatcgccgacgcggtcggcgcgcccgtcgcgaccgtcggggaactgctgctgcgcggtatcagcgccgagcagctcgcgcgggccggcaccgaccgcccggacgcgctcttccggctggagtgggaggcgctgcgcggcgacggggcgaccaccgcgggggagtgggcgctgctcggaggggacccgtacggtctcgctccggcccgtgccgtcccgtacggcgatctcgacgccctggcggccgaggccgagggcggcacgccgcccgaggtcgtcctgctgcccctgacagtgcccgccggcgaagagccggacgcgggcgagcgggccgcgcaccgggcgctgcgcgcggtgcggtcctggctcgccgacgaccggttcgcggctgcgaccctcgtcctgatgaccaggggcgcgctggcggcgacccccggcgatgaggtcaccgatgtggcgggcgccgaggtctggggcctcgtacggtcggcgcagaccgagcaccccggccggttcgtcctggtcgactcggacggttccgaggcgtccgcgcgggcgctcccggccgccgtggcgagcggtgagccacagctcgcactgcgcgcgggtaccgtcaacgcggcccggctgggccgcgtggagcgggccggggcggaggccgtggccacgttcgatcccgagcggaccgtcctgatcaccgggggtacgggcgcgctcgccggccaactcgcccggcatctcgcccgtgcgtacggcgtccggcatctgctgctcgccgggcggcgcggcccgagcgccccccacgcggcggagctggtcgccgagctggccgaactgggcgccctggccgaggtcgtggcctgcgatgtggccgaccgtgaggcgctgaccgccctgctggccgcggtccccgccgaccggccgctgggtgccgtcgtgcacacggcgggggtcctggacgacgggctcgtggagtccctcacgcccgagcggctcgacgcggtgctccacccgaaggcgaccgcggccctcctgctcgacgaactgacccgtgacgccgacctgacggcgttcgtgctcttctcctccgccgcgggcaccctcggcagccccggtcaggccaactacgcggcggccaacgccggtctcgacgcgctcgccgtccggcgacgcggccaggggctgcccggtctgtcgctcggctggggcctgtggcaggcggacggcggtatgggcggcgcgctcagcggcggcgaccaggcgcgtatcgcccgtggtggtgtcgccgcgctcacgaccgaccacggtctggcgctcttcgacaccgcctgcgccggtccggacgccgtcgtactgccgatgctgctggacctgcgaccgcaggacgacgtaccgcatctgctgcggtccctggtgagcgcgcgccgcaagggcgcgtcggcgggagcccgccacgagggtccggccgagctgcggcgcaggctcgccgccgcgacgcccgacgagcggtacgaacacctcctcggcctggtccgctcgtgcaccgccgtggtgctggggcaccggggcccgcaggacgtcgatcccggggtcgggttcctggagtccggtttcgactcgctgaccgcgatggagctgcgcaaccagctcaacgaggccaccggtctgcggctcgcggccaccgtcgtcttcgaccacaccacccccgcagatctggcgcggcacctggtcgacggagtggccgacacggtcgccgccgcaccgggccagcagccggcgccgcggtcggacgacgacgcggacaccctgagcgggctgttccgtacggcggtacgggccggtcaggtgacgaagggcgtcgatctgctacgggccgtggccgagctgcgcccggccttccacagcgccgacgaactgggcgacgatctgccggtgccggtacggctcgcccagggcccgagtgccgcgcggctgttctgcttcgcctcgccgatggcgatgggcggcacgcaccagtacgccaggctggcggcgcacttccggggcgtacgcgaggtctcggcgctccccatgcccggattcgcgacgggagacctgctgccggcgacggccgaagccgtcgtggaggtcttcggccggagcctcctgcgggcggccggggacgagccgttcgtcctcctcggctactcggctggcggtgtcttcgcccacgcggtggccgcctggctggagtccgaagggtgcgcgccagccgccgtggtgctgctggacagctaccgcgcggacggtggcaccagcatggacggcgacttctgggtgagcatggtggagggcctgttctcgcgggaagaggtcttcggacggttcaccagcgcccgactgtcggcgatgggccggtacgcgcggctcgtcggcgaggtcaagacgggtgaggtgaaggcgccggtgctcttcgtccgcccggagcggtcgctgagcgcgagcgccgacgcgggcggagtcgggggcggggacgccggggccgctgccgccacggatgactggcgggcctcgtgggacacggccgagacggtgctggaggtggcgggcgaccacttcgacatcatcgagagccaggccgccgcgaccgccgacgccgtggagggatggctcgcggaccgggtctgacccgtctccccatcagccggtcgagcgccggaggacatcgctgagggccgaccccgcacaggggccggccctcggccgtaggtacgggtgggacgcgtacccgccgtccgcatcgcgtgccttgctcgtaccgcgacctgctcgtaccgcgacctgctcgtaccgtgacccctgcctgtacgcgccctgcccgcactgcgatcctgcccgcactgcgatcctgcccgcactgcgatcctgcccgtacgcgccgtgcccgtacgccccctgcccataccgcacttgtccgtacgcgccccgcctcccgtacgagcgcgcgcgggaacagcattcccgtacgacgtcaggggcggtgcggcgaggatcccggcccggaagccgttgtccttgccgcaccgcccctgacggtgcggagcgcgtcagcctatggctgtcgcacccccacgaagacaccgcggtcgacatacccgtcgggcaggtactccaccgagcacccggccgcgcggaacgcctcctcgtactcgtccttggagaacagcgacagctcctgttcgtccacgaagtgccgtacgggatcgtcgccgctcgtcaccaggtagtgcatctccacatggctgcgcccggcccgggtggtccagcggcccatgcggaccaccttgccgccctcgttctcgaagctcgcctccaccagatcgccgccgttccaactgtcccgcagaatccacggctcgatgatcagcaccccgccgggacgcagatggtgggccatgttcttcagggcggcgaacagaccgtccagggtctccagatagccgacggagctgtacatcgaacagatcacgtcgaagctccggtcgagcccgaaggtgcgcatatcgccctcgtggaccggcacccccggcagcttggcctcggcgacccggaccatcggcgccgacaggtccacaccggtgacggcgaaatcgtccctcagcagcgcgaggtgctcgccggtgccacagcccacatcgagcagatcggccgctccggggttgcgttcgagcgccagctcgcggatgcgggcggcctcaccggcgtagtccttgtgccgccgatagatgtcgtcatagacgcgcgcgaagtcctcttcgtacatgaaggagtcacctggccggggttcgggcactgacacagcgccggatgatgtcgcagacctgatcgatctccgacggcccgacggtggtacccgtcgggagggcgagcacgcgctcggccagtgcctccgcgtgcggcagagggtgtggcgcgtgccgggcggggtcgctcagatagggctccacctgatggcaactggggtggaagtagcggcgcgagaggacgttctgggcgatgagggcggtgtgcacctcgtcgcggtggatacccgccactgcggcgtcgatctcgatcaccagatactggtggttggcgcgctcgccctcggcctgaccgcgtacccagacaccgggcagcccgtccaggcccttctcgtagcggccgtggttgatccggttggcctccatgaggctgtccatcacctccagcgaggtgagacccatggccgccgctgcttcgttcatcttggcgttggtcccgccggagcggatctcaccggcgctggtgatgccgaagttgcgcagctcgcggcagcgctcggcgaactcggcgtcccgggtgacgatcgcgccgccctcgaagctgttgacgaacttggtcgcgtggaagctgtacacctccgccgtgccgaagccgccgacggggcggcccttgtaggtgcagccgatggcgtgcgcggagtcgaacagcagcggcagtccgtgccggccggcgatctcctccagctcgtccacctcgcaggggcggccgaagacgtgcacgcccatgatgccgcgggtgcgcgggccgatcagcgcctcgaccttggtccagtcgatgtttccggtcgtctcgtcggcgtcgcagaagacggggacgattccgatccagtccagtgccgccgcggtggccacccaggtgaaggccgggacgatgacctcgtcgccaccccggattcccgcggccttcgccgccacctggagcccgatcgtggcgttgcacaccgcgacgcagtggtcgacaccggcgacctcggcgagtctctcctcgaattctcggaccagcggaccattggtgagatagagccggtcgagtgctccctccagtctctccgtcaggcgggcacgactgcctatattgggtcgcccgacatgcaacggctcgcggaacacaggggaatcagtcacctgttcatcgttccgttcacccgcccctagcctcaacctgcgggcatgggcgcgccccagtaggggccgccgccagcccctaaccacccccctatgcggtcccactgctgctgacgcggcggcctccgcggcactagcgtcggcgccgtctgccactgagttcaccatggccagccgccccaatggagatttccatgaccgagacgctgtcgactcttccgcccacccacaccggcgacgagtcgctcgcggtccgtatcgccaagtcggtgagtgtcgtcgacgacggggcgctgcacagcctcgacgaattccacgactggttcaccgccagagggcagcggaccgcacatgtcgagagagtgccgctcgacgaactcactggttggagctccgatcccgtgaccgggaacatcgggcacgacagtgggaagttcttcagtgtgcaggggctctccgtcgaactgcccggtgcgccggtgcccgcctgggcgcagcccatcatcaatcagcccgagatcgggatcctggggattctgatcaaggaattcaacggtgttctgcactgtctgatgcaggcgaagctggagccggggaaccgcaacggcctccagctctccccgacggtgcaggcgacccggagcaactacacccgggtgcaccagggcaaccccgtcccgtacgtgggctacttccaggacacctcggcgaaccgggtgatcaccgacgcgctccagtccgaacagggctcgtggttctaccagaagcgcaaccggaacatggtcgtcgagaccgaggcggagatcgagcagcacgaggcgttcatgtggctgaccatcggtcagctgcaccgcctccttgcgatcgacgacctgatcaacatggacacccgtacggtgctgtcgtgtctgcccttctccggtgcgcagctcgccgagcagctcccgggcaccggcggcgatctgcggatgccgatcctgcgctcgtgcagcgaggaccagggcagtctgcacaccaccggggacctgctgagctggatcactgacgcccgtacccgcaacgaggtgcgcacccggctgacgccgctgcgcgacgtggcgggctggcggcgcaccccggacgcgatcacgcacgacacgggtcggttcttcgacgtgatggccgtgcgcatcgagaccgacggggaccgcgaggtccgccagtggacccagccgatgatcgcgccggccgggatcggcatcgtggcgttcctggtcaagcggatcgacggggtgctgcacgtgctggcgcacgcccgggtcgagccgggctatctcgatgtcgtggagctgtcgcccacggtcatgtgcacgcccggcaactacgagggcctgcccgccgccgcccggccgccgttcctcggcgaggtgctctcggcccgtcccgagcaggtccgcttcgagacgatcctctccgaggagggcggccggttctaccacgcgcagaaccggtacatcatcgtcgagtccgatatcgacgtggcgccggaactggcctcggagtaccgctggatggcgctccaccagatggtcggcctgctgcggcacagccactacgtcaacgtccaggcccgcagtctgatcgcgtgtctgcacagcctctcgggggcgcaggggcggaactgacccgcgccctccccggatctcctcccgctctccccggccctgaaagggtgacgatgctctaccggcagctcggacgtacggcactgaaggtcagcccgctctgtctgggcaccctcaacctcggcgtccgtaccacccgtgacgaggcgttcgccctcatggacgaggccctggagcagggcatcaacttcttcgacaccgccaaccagtacggctggcaggtccacaagggcctgacggaggagatcatcggagagtggttcgcacagggcggcggccggcgcgagcgcgtggtcctgggcaccaaggtctgcaacccgatgagcgatctgcccaacgaccaggggctgtcggcgcggcacatcatcgcctcctgcgaggactcgctacggcggctgcgcaccgactggatcgatgtctaccagctccacaacatcgacccgacggcgtcctgggacgaggtgtggcaggccatggagaccctggtgcaccagggcaagatccgttacgtggggtcgtccaacttcgcgggctggcatctggccgacgcgcaggccgcggcggcccgccgtaacttcctcggactcgtgtcggagcagtgctgctacaacctggcgacccggtacgtggagatggaggtcgtccccgccgcgcgggcgcacggcatcggagtgctggcctggtcgccgctgcacggcggtctgctgagcggggcgctgcgcaagctggacgaggggacggcggtgaagacggcccagggccgtgcgcagatcgcgctgcggaccatgcgggacaccgtcgagcagtacgagaagctgtgcgagagcatctccgccgacccgtcccaggttgccctggcctggctgctgtcccggccgggtctcacggccgccgtcatcggcccgcgcaccaccgggcacctggcgagcgcgctggagaccctggacatgacgctgcccgaggacatcctggcgccgctggacgaactcttcccgccgatcggcaacggtcagccggcgccggccacctggctcgcctgacgggcgggacgacttccccggccggtcacccacgagcgtgtcaccggccggggggcaccagcggtcgggagccctgacaacgggcaccgccactggtgccggacccgaacaacgaccgcgtcccggccgttcccacccctgacacggagcagtccccacatgccgcctcatgtccgcccgatgcccgacatcacatccgaacggctcccggaccaggaccaccacctgcccgccgagcgtgcccggcccgtgctgatcagcagcgtcgcctccgactcgcacacctggaacctggtcttcctccaactgctgatcgaggagctgggacacgaggtcatcaacctcggtccctgcgtgcccgacgaactgctcatcgccgagtgccgtgaccgccgccccggcctcgtcgtcatcagcaccgtcaacggccacggctaccaggacgggctacgggtgatcgggaaactgcgcgcctgcgaggacctcgcgaacatccccgtcgtcatcggcgggaagctcggcgtctcgggcccgggtcagtcgtacgcggccgagttggccggcgccgggttcgacgcggtgttccccgacggggcggaggccgtgtcctccttcaccagatacgtcgagaagctcccgcagcgagtagtgtcttgaccggattcggcgcctacgtccgctccgtccaccggcgcggcgaactcgtcgtccagccgcggatgggattcagcggtacggtcgagatgcgcgccggtctcgccgcgacccgggcggcggacgcggccacggtcggcaccatcaccctggacagctacacccgggtcggggaactcgccgcagccgaggaggcactccgctccggcatcgccctcaacggctaccccatcgtcacctacgacgaatccaccacccgtgaactcctcgccgggatcgcgggtgaggacttccccgttcagatccggcacgggtcggcgacccccctgcacatcttcggcgcgctgacccgcgcgggcctggacgccaccgagggcggccccgtctcctactgcctgccctacggccggattccgctggaacagtcggtgaccaactggacccgctgctgcgaggagttcgcccggctgcgcgggctgggccgtgaaccgcatctggagaccttcggcggctgcatgctcgggcagctctgcccgcccagccagctcgtcgccatcagcatgctcgaagcgctcttcttccaccagcacggcattcgcagcatctccctcagctacgcccagcagacccatcagggacaggacgaggaagccgtgctggcgctgcgccggctccaccgcgaactgctgcccgatgccgactgccacatcgtcatctacgcctatatgggggtctatccgaccacccccgagggggcccacgcactgctgggccgggccgccgaactcgccgtcgccaccggggccgagcggctcatcgtcaagaccgcggccgaggcccaccgcatccccaccatcggggagaacgtggcggccctggagcacgcggccaccgtgtcccgcgccgcccgccgggccaccgcgctcccggccggtcactccgctgcgcccgctgccaccgaccccaccgccgcgctcacggcccgacccggtacgtacgcggacggatccgccgcgctcacagcgggccccggcgccgcgcgggacggctccaccgactccgccgtctacgcggaggcccgcgccctcatcgaagccgtcatgaactgcggaccggatgtcggcaccgcactgttccgcgctttcaagaggggatacctggacatcccctactgcctccaccccgacaacatgggccgcagccggagttacatcgacgacaccgggcggctgcaatgggcggagaccggaatgctgcccctggggaatgccagaaaaagcggaacaggccgctcggtatcgtcaaccgatctgatatcggccctttccttcgtgcagcggacctatgaccaactggcatttccggaatcccgggagccgctccggataccccggcagcagcgcccctaggggtggctagggggcggataggggacggctggagcggcgctctcccctagcatgagcgacgtaatcgaaccgtgaggtggtgtgaatgtccaagcccccgagtgccaaggggactgtgccgttcggtcaataccggacgtggtatcgggtaacgggcgatctgcattccggtaaaccaccggtggtgctgttgcacggtggtcccggctccacccacgactacctgctggccatgacctccctcaccgaagccggctggcccgtcgtccactacgaccagctcggcaacggcggatccacccacctccccgagaagggcgaggacttctggaccgtccagctcttcgaggacgaactggacaacctcctcaaccagctcggcatagccggcgactacgtgctcttcggccagtcctggggcgggatgctcggctcggtgcacgccgcccgccgccccgccggactgcgcggcctcgtcgtcgccaacgcacccgcctccatgaagatctggctccaggagatggcgcggctgcgcgcactgctcccgcccgacgtgcaggagaccctcctcaagcacgaggcggcgcggaccacggacaccgaggagtacttccacgccatgcgggccttctacgaccggcacgtctgccggatcgtgccctggccccgggacttcgccgccaccttcatggagatctacaacgacccgacggtctacacgaccatgaacgggccgaacgagttccatgtgatcggcaccctgcgcgactggtccgtcgaggactgcctgcccgacatccaggtgcccaccatggtcctcatcggccgtcatgacgaggccacccccgcgaccgtcaagcccttcctcgacctcgtgcccgacgtccggtacgaggtgctggagaactccagccacgtaccgcacctggaggagcccgagcggttccacgaggtgatgatcgactacctcgaaagcctggtgtgaacgcgaccgtggagacgacacagcacgacgtcgaggggaccggcgcggccggcgcgaccgcgatgctcttccccgggatgggacccgccgcgttctccgacgtcggccggttcatggtgaccaaccggtacaccagggaactcctcgcggaggccgacgacaccctcggctactccctcgtcgaccggttccggcaggccgagggcgactactccgagtacgcccagatcgccttcctggtcaactgcgtcgccctcgcccgctgggccgagcagaccatggacctcaccccccggatctgcgccggagccagcttcggcgagaagtccgtcgccgcctacagcggcgccctcaccttcgccgacgccgtccggatgaccgccggcctcgcccgctgtatggacgagtacttccgtacggagcacctcggggtcgtcacccactccttcgtccgcgcccctcgggagcggctcgacgagatcctcgccgaactcgacgagcgcggcgaatggcacgagatctcctgccacatcgaccacgacttcttcatgctcaccctccatgaacgcaacagtgtctggctggagggccggctccggtcggtgggcgccatgccgctgtacgccatgcggcccccgatgcacgccgccgccttcggcgggctgcgcgacaaggccgaggaggaggtcatcgcccccctcaccttccacgacccgaccctgcccgtcgtcgccgaccaggacggcaaggtgctgaccaccggcgacgaggtgcgcaccatgctcctggagagcttcgtccgcccgctgcgctggcccgacgtcatctcctcgctccaggaccagggcgtcacccgcgtctgtgtcgcgggccccgacagcctcttcgggcgggtcggcaccaccacccgcgccttcgaggtcatcgcggcgaccccgcggctcgccctccagccccgcgcgcgcaccacctcgcgctgaccccgatcggggttccgcccgcccaccacggcggcgggaccccgggggtccccgcaccacggccgcccggccaccaccaccgtccgcccccctgccgggggccgaagggagcctcccatgtgggacgcccagttcgagaacctcctccgccgctacctcccgttcctctccgccgaccagccgctggagcaggacatcaacctgcgtgacatcggcctcgactcgctcggcaccgtcgagctgctctccgagctggagaacacctacgacgtccacttccaggacgaggcgctcaccaaggagaccttcgagacccccggcgtgctctggaagaccctctcccagatggtcgagccgcgccactgaccacccggcccggcgcacaacccgggccgccagcccggcccgacggaggaccgatgcacgccgccgaccacgccctgcacgcccgcttcctgcgcgggctcgcctgcgcgcccgaccggcccgccgtacggttcggcgggcggaccctgacgtacgcgcaggcccaccgcaccgccctgacctgggcgggttccctgctgcgcgccacgccggagcccccggccgccgtcggtgtcctcgccgacaagggcatccccgcctatctgggcatcctgaccgcgctctacgccggcgccgccgtcgtgccgctgcggccggacttccccgcggcccgtaccgccgagatgatgcgggccgcgggggtcaccgccgtcatagccgacggtcgtggccggcggctgctccccgaactgctcgccgaccgccgggacaccgccgtcctggccgccgacgaggaaggcgcccccgccgacgagagccccgccgacgggagcgccccgggccgccgtgtcgcgatcgacgaggggtacgcgctcaccgcgccccgggacgtcgtcccggacgacaccgcgtacgtcctcttcacctccggctccaccgggcggcccaagggcgtgccgctcagccacggcaacatcgcccactacttcgaggtcctcgacgcccgctacgacttcaccgcggacgacgtcttcacccagaccttcgacctcaacttcgactgctcgctcttcgacctgttctgcgcctggggcgccggcgcgagcgtcatccagatcccgccccaggcataccgggacctgccgtcccacctcgccgaacagggcgtcaccgtctggttctccaccccgagcagcatcgcgctggtgcgccggctcggcggtctggcacccggctcactgcccaccctgcgctggagcttcttcgcgggcgaggcactcaagtgcgccgacacggaggactggcagcgcgccgcgcccgcctccttcgtggagaacctgtacggccccaccgaactcaccgtcaccgtcaccgcccaccgctggtccccggaggtctccccggtcgtcggcgccaacggcgtcgtccccatcggccccctccacaagggcctggaccacgtcctgatcgacgccggtggactgccccaccccgacaccggggaactctgcgtcaccggaccccagatggcgggccgctacctcgaccccgccgacgaccacggccgcttcctcgaccacgacgggcgccgctggtaccgcaccggggaccgggtccgactggcccccggcggggaactcgtctacctcggccggatggacgcccaggtccagatccagggctggcgggtggagctcgccgaggtcgatcacgccctccaggggtgcgagggcgtcggcgaggccgtcaccgtcggcgccgccaccgacgcgggcaccgagctggtcgtcttctacacggcccccgccccggtgccgcccgtccgcttcgccgccgtgctgcgcgccaccctgcccgacggtgtcgtaccacggcactaccgccatgtcgccgaactgccgttgaattccaaccgcaagatcgaccggcgagcactgaccgcacgtgccgaggaactcctcggctgagcacgtccgagcggccgggcaaccccccggccgctcactcattgccgcaccccttacagcgacaggcccctaggggccgctaggggtatgtcggccggcccgcaccgggctagcctcactattgacagacgtattcgggggagttcgccgtgtgtattcaccagacaggagagcggcgttgaacagcgtggccaacatggtgtccgacaatgccgataaggatctccgctacggcggactcgtccacgatctcctggcagactcaggaaaagcgacaccgaattccgatgccatggaagacgcattcggaacctggacctatcaagagctgctgaaccatagtcaggcattcagcgcctggctggacggaaagggcgtcgcccgcggcgaacgcatcgtcgtccagctcccgaacatccgccagacggtcgccgtgttctacggagcctgccgccgcggcgtcgtgttcgtcccgctcaacccgggcatgaagcccttccacctgcggtccgtcatcgccgacgccgacccgcgcctggtcatcgccgaggacgagaccgcggccgaccggctgcgtgacgtcaccgacctgccggtgtactccatcgactccctctgggcggatgtcgagcggctgcgcgacgcgggcgccggagccgaggccgtcgaggtctccccggaggatctggccgtcctcatctacacctccggctccaccgcggcccccaaggcggtcgcctgcccccaccagcagatcgtgttcgccgcctcctcgatcaacgccgtactcggctaccacgccgaagacatcgtcttctgccggatgtcggtctcctgggacttcggtctctacaaggtgctgatctccaccctcaccggcgccaagctcgtcctcgcgggcggtgagcccgacatcgcgctcgtcaagtccctccgcgagagcggcgccacgatgatgcccatcgtgccgtccctggcgagcatgctcaccaccctcatccggcgcgaccccgagggcgccccgacgctgcggatgttcaccaacagcgccgccgcgctgccccaggtcaccatcgacgccctgcgctccgcgttccccggcgcccaggtcgtgcgcatgtacggccagaccgagtgcaagcgcatctcgatcatgccgccgcatctggagcacgagcgacccgactccgtcggtctgccgctgcccggcaccaccatcgagatcctggacgaggacggcaccctcctgccgcccggtgagcccggcgagatcaccgtcaccggaccccatgtcatggccggctactggcgcgcgcccgagatcaccgcccgcgcctaccgccgcgacgagaccaccggcgcgatgcgcctgcacaccggcgactacggccacctcgacgaggacggattcctctacttcggcggccggcgcgacgacatgttcaagcgcaagggcacccggatgagcaccgtcgagatcgaggccgcggccctcgacatccccggcgtcaccgccgccgtggcgctgcccccgaccgccacccgggacctggccctgtgtgtcgcgtccgacctcgaaccccatgacgtcctccgcagcctcgcggagcggctcgaaccggcgaaggtccccgcgacctgccgtatcgtcaacgacttcccgctcaccctgaacggcaagtccgagcgcaagcagctcgcacgcctgctcgacgggagcgacaagtgaaccagtacgaagaactcgccgaccagtacggcaccccgctctacgtctacgacctcgaccgcgtcgccgaggcccggcacgatctgcgcacctcactgcccgacgaggtcgagatctactacgccctcaaggccaacccccacccgcaggtcgccggcgccctgcgcagcggcgagggccgcgagtgccgcgccgagatcagctccgtcggcgagctggccgccgccctcaccgccggcttccgtgcctccgagatcctctacaccggccccggcaagaccgacggcgaactggacgaagcgatcggcaagggcgtcaagaccttctgcgtcgaatcgctgaccgacctccagcacgtcggcgccgtcgccctgcgacacggcgtcgtcgccgactgcctgctgcgcatcaacagcgccaccgccagcgccaccaccagcatccggatgaccggaaccccctcacagttcggcatcgacagcgagaccctcgtcgacgccatgcccgaactgcgcgccgtacccggcacccggatcacgggcctgcacttcttccccctcagcaacgcgagggacgaggccagcctcatcggcgagttccgccacaccatcgcctacgcggccggcctcgccgaggagaccggtctcaccctggaattcctcgacatcggcggcggcttcgcccacccctacggcgcgcccggcgagcgccccgtctaccgcgaactccgcaccgagctggccgccgccctcgacgagcacttcccgcactggcgcgagggcgccccgcgcatcgcggtggagaccggccgctaccagaccagcggcgccggaaccctgctcacccgggtcgtcaacatcaaggtcagccggggccggaagttcgtcgtcatcgacgccgggatcaacaccttcggcggtatgtccggcctcggccggctgctgccggtcgccgtcgaacccgagtacaccggctccgccgaagccaccgaactcaccgatgtcgccagcctcgccggccccctctgcacccccggtgacatcctcggccgcgagatccgcctgcccgaactggcccccggcgacctcgtgaccatccccaacgcgggcgcgtacggcgtcaccgcgagcctgctgatgttcctcggcaggcccgcgccggtcgaggtcgtcctgaagggcggcaaggtcgtctccgcgtcccgactggagcaccaccgcactccggcgacccccggataaccacggccgaccacccgccgcgcccccggtgacctccgggggcggccggtaccgagccccctgcgcaccacatccgggaacgcccgccacgggcgttcccgccttttcccgcgccgtcgccaccgagcccgcgcgcgccgcaccacgtatgtcgcccgccttagcgaggagtaagcgaatgagcgagtccgagaaccccgtttccgccaccacagccggtgcggatgaaaccccggacacgcgtacggcgctggctcggcggctggcgggactgtcccccgccgagcaggagcagcacctcgtcgacatggtgcaccggcacacggtcgccgcgctccaggccgtcgccccgctcactccggaccaggtcgatgtgcagcgcccgttcctggaactcggcttcgactccctcgccgccgtggacctgcacaagcggctcaccggcgagaccggcctcgaactgccggtgaccgtcgccttcgacttccccacccccgtactcgtcgccgaggagatccgccggatcgccttcggcatccggcccgccccgctcgcgcccgtcgtcgccaccggcaacctggacgacgaccccatcgcgatcatcggcatcggctgccgtttccccggcggcatcaactccgccgaggaactgtggcagctcgtcatggacgaggccgaggtgctctccggattccccaccaaccgtggctgggacgtcgagggcatctacgaccccgacccgggcaagcccggcaagagctacgtccgcgaaggaggcttcctccaggacgcgggcgacttcgacgcggacttcttcgggatctcgccgcgcgaggccctcgccatggacccgcagcagcggctcgtcctggagaccgcctgggaggcgttcgaacgcgcgggcatcgaccccggctccctccacggcagcaaggccggtgtcttcatcggcgccgaggtccatgagtacggtacgcgcgtccatgaagcccccgaagggctcgacggctacctcatgaccggcaacgcgcccagcgtcgcctccggccggatcgcctacagcctcgggctcgaaggccccgccgtcaccatcgacaccgcctgctcgggctccctcgtcgccctgcacctcgccgcgcactcgctgcgccagggcgagtcctcgctcgccatcgccggcggtgtgaccgtcatgggcaaccccggcatgttcgccgccttcagccggcagcgcggtctcgccgccgacggccgctgcaagcccttcgccgccgccgccgacggcaccggcttctccgagggcgtcggcgtcttcgtcgttgagcgcctcgccgacgcccgccgcaacggccaccccgtgctgggcatcgtcaagggctccgccatcaaccaggacggcgccagcaacggactgaccgccccgaacggcccctcgcagcagcgcctcatcctccaggccctcgccaacgccggtctgaagccggcggacgtcgacaccatggaggcccacggcaccggcaccaagctgggtgacccgatcgaggcccaggccatcctggcgacctacgggcaggagcgcgaagagggcaggccgctctggctcggctccatcaagtccaacctcggccacacccaggccgcgggcggcgccgccagcctcatcaagatgctgatggggatgcgccacggcaagctgccgcgcagcctccacatcgacgcgcccaccccgcacgtcgactggacgaccggtgacgtcgcgctgctcaccgagacccgcgactgggacgcccccgaccgcccccggcgcgccggtgtctccgccttcggcatcagcggcaccaacgcgcacgtgatcatcgaggaggcgccggagttcgagccgagcgccgtggagccggtggccgggtcgggggtgacaccgccgtgggtgctgtcggcccggtcggccgacgcgttgcgggggcaggccgagcggctgctgtcgttcgtctccaccgacgccgatgtctccgtggtggatgtggcgtactcactcggtgtgtcgcgtgcgggcctggaacaccgcggtgtggtgctgggggagtcgcgcgaggaactgatcgcggctctggagtcgctggcgtccggtgccgaggcgccgggtgtggtgacgggtcgggtcgccgagggccggttggcgttcctgttcaccggccagggtgcgcagcgcgtcggtatgggccgggaactcgccgccgaattccccgtgttcgccgcttccctggaagagacctgcgacctgctggacgtgggcctggaggaccaggaccactcactgcgcgaggtgctgttcgccgaggagggctcggccgaggccgccctgctgacgcggacggtgtacgcccaggccgcgttgttcgcggtggaggtggcgctgttccggctggtggagtcgttcggtgtggtgccggacttcgtcgccggtcactcggtgggtgagatcgccgccgcacacgtcgcgggcgtgttctcgctggaagacgccaccatgctcgtcgccgcccggggccggctgatggacgccctgccggagggcgggacgatggtggcggtgcaggccaccgaggaggatgtgctcgccctgctggagggggtggaggacgcctccatcgccgcgatcaacggccccgacgccgtggtcgtctccggcaccgagaccggtgtcgcccgggtggtcgacgtactccgggaccggggcgcgaagaccaagcggctcgacgtcagccacgccttccactcaccgctcatggagccgatgctcaccgagttccgccgggtggccgaggtcctcgaataccaggcaccgcggatcgccgtggtgtcgaacgtgtccggatcggtcgccggcaccgaactggccacgcccgagtactgggtcacccacgtccgcgaggccgtccgcttcagcgccggcgtccggaccctgctcggcgccggcgtctcctccttcctggagatcggtccggacggtgtgctcagcgggatggcccgtgggtcggtctccgacggcgccgacgttggctgtgtccccgtgatgcgccggggccggggtgaggtgcgggagttcctgaccggcctctcccggatcgccgtacgcggagtcccggtcacctgggagccgctgatcgaaggcgcccgccgcgtcgagctgcccacctacgccttccagcgccgctggttctggctggaggccggccgctcgaccacggacgccctgggcctgggccagaccgccgcggagcaccccttggtgggtgccgtggtgggtctggccggtggtgatggtgtcgtcctgaccggtcgggtgtcgttgcacacccacccctggttggcggatcaccaggtcgccggggtgacgctgctgccgggtacgggcttcgtggagctggcggtccgtgccggtgacgaggtcggctgtggccggttggaggaactgacgctggaggcaccgctcgtcgtgcccgaccggggcgccgtccagctccaggtcgtcgtcggcgcactggaggagtccgacgtccgcaccgtcagcgtctactcccgcgccgaggaggacgacgtctggacccgccacgccaccggattcgtcggcgccgccgtcgacggcggactgaacctggaggcatggcccccgccgggcgcccgggcggtggacgtcagcgacgtctacgccggactggccgaccagggctacggatacggtcccgtcttccagggcctccggtcggtgtggcgcggcgacggcgaggtgttcgccgaggtcgtgttgccggaaggtgcacaggcggacgcctccgcattcgggctgcaccccgccctgctggacgccgcactgcacgccaccgactacctcgacccggagtccgccgtcgagggcacgcatctgccgttcgcctggaccggtgtctcgctgcacgccaccggtgcgtccgaactgcgggtacggatcaccgccaccggcaacgacggatacgcgctcgacctcgccgacaccaccggccgccccgtcgccaccgtccagtcgctcgtgctgcgcccggtcaccgaggagcagctccgcagcgcccagggcggcaacggccccgacagcctgttccgggtggaatgggcccccgtcaccgccgacctcaccgcatccggcgccggctgggccgtgctcggcgccggtgctcccgaactcgccgccgccctcaccgcggccggcaccccgggcaccgcctaccacggcacggccgagctgagcgccgccgtcgacggtggcaccacccccgatgtcgtcgccgtccagttgccgtcggccggcgccgccgacgccgacctcccggacgcagtccgcgccaccctgaacaccgcgctggagttcgtccagaactggctggccgacgagcgtctcgccgagaccaggctcgtggtcgtcacccgcgacgccgtcaccgtcgacaccgccgcgggcggccccgacctgaccgtcgcccccgtctggggactgatccgctccgcgcaggccgagaaccccggccgcatcctgctcgccgacatcgacggcaccgaccagtcccgtaccgccctggccgcgatcaccgccgccgacgaacccgaactcgccttccgcaacggcaacgcgtacgccccccggctggtcaggtccgtcagcgagggcggcctcgtcccgccgtcggacgccgcgagctggcgcctggacgtgctgagccagggcaccctggagaacctcgccctcatccccagcgacgccgacgagcggaccctcgccccgggcgaggtccggatcgccgtccgtgccgcgggagtcaacttccgtgacgtcctggtggcgctcgggatgtacccgaccaaggcggacatcggtggtgaggccgccggtctcgtactcgaagtcggccccggtgtcaccgacttcacccccggcgaccgggtgatgggtctcttcgacaccgccttcggtccgcacgcgatcaccgaccaccgcaccctggtgccgatgcccaccggctggacctacgcgcaggccgccacggccccgttggtcttcgccaccgcgtactacggcctcgtggacctcgccgaactgcgctccggcgaatcggcgctcatccacgccgcggccggcggcgtcggcatggcggccgtccagatcgcccgccacctgggcgccgaggtgtacggcacggccagccccggcaagtggaacgcgctgcgcgccgccggactcgacgacaaccacatcgcgtcgtcccgtgacaccacgttcgagcagaagttcctcgcgaactccggtggccgtggcgtcgatgtcgtcctggacgccctcaccggcgaattcatcgacgcgtcactccggctgctgccgcgcggtggacgcttcgccgagatgggcaagaccgatgtgcgcgacccggagcgggtcgcggccgagtaccccggagtgcgctaccgggccttcgacctcttcgaggcggacctcgaccggctccgcgagatcctgcgcgagctgctcgccctcttcgagagcggtgcgctgcggccgctgccggtgcgtgcatgggacatccgcaagtccaaggacgccttccgccatatcgcccaggcccggcacatcggcaaggtcgccctcaccatgcccacgggtggtttgggtgatggtgtggtgttggtgacgggtggtacgggtgggttgggtgcgttggtggcgcggcatgtggtggtggtgcatggggtgcgtcgtttggtgttggtgtcgcgtcgtgggttgggtgctccgggtgcggtggggttggtggctgagttggagggtttgggtgctgtggtggaggtggtggcgtgtgatgtgagtgatcgtgtggcgttggcgggtgtggtggggggtattgggtcggatctgtcggctgtggtgcatacggcgggtgtggtggatgacggtgtggtgggttcgttgtcggtggggcggttgtcgtcggtgttgggtccgaaggcggatgcggcgtggtatttgcatgagttgacggtggggttggatctgtcggcgtttgtgttgttctcgtcggttgctggtgtggtggatggtgcgggtcaggggaattatgcggcggcgaatgtgtttttggatgggttggctgtggtgcggcgtggtttggggttgccgggtacgtcggtggcgtgggggttgtgggagggggctggtatgggtgcggtgttgggtgaggctgatgtgttgcggatgagtcgttcgggtgtgttggggttgtcggtgggtgaggggttggggttgtttgatgtggcgttgggtgaggatgtggcggcgttggtgccggtgcggttggatttgggggctttgcgttcgcgggttgatggggtgcctgcggtgttccggtcgttggtgcgggtgccggtgcgtcgttcggtgggtgtgggtgtttcgggtggtggtgaggtgtcgttggagcggcgtttggtggtgttggatgcgggtgagcgtgagcgggtgttgttggagttggtgcggtcgcatgtggcggctgtgttgggttatgagggtgcggggtcgattgagccgggtcgtgcgttcagtgatatcgggttcgattcgttgtcggcggttgagctgcggaatcgtttgaatggtgagacggggctgcggctgccggcgacgctgatcttcgattaccccacccctcaagccctcgccgaatacgtcatcgtcgccctgctcggcgaggaggcggccaacgcccccgccctgccgcccaccgaggcccccaccgtcaccggaaccgatgacgaccccgtggtcatcgtcggcatgggctgccgcttccccggcgacgtccgtacccccgaggacctgtggcgactggtctccaacggcagcgacgccgtgacccccttccccgacaaccgcgcctgggacatcgaaggcatctacgacccgctgcccggcgtgtccgggaagacgtacgcccgcgagggcggcttcctgcacgacgcggccgagttcgaccccgagttcttcgggatctcgccgcgtgaggccctcgccatggacccccagcagcgactgctccttgagacgtcctgggaggccatcgagcgggcgggcatcgacccgaacagcctgcgcggaacccagaccggcgtcttcgccggagtcatgcagaccgactacggcaacggcggcgcccggctcgccgaggacgtcgagggctacatcgccaacggcacgctcggcagcatcgtctccggccgggtctcctacgccctgggtctggagggcccggcggtcacgatcgacaccgcctgctcctcctccctcgttgccatgcactgggcggcccacgccctgcggcagggcgagtgctcgctcgccctggccggcggcgtgaccgtcatgtcgacgccggagaccttcgtcgacttcagccttcagcgcggcctcgctcccaacggtcggtgcaaggcgttctcggcggacgccgacggcaccggctggggcgagggcgtcggcgtcctggtccttgagcggttgtcggacgcccgccggaacgggcatcaggtactcgcggtcatccgtggttccgcgctgaaccaggacggtgcgagcaacggcctgacggctccgaatggtccctcgcagcagcgggtgatccgccaggccctcgtgagcgccggtctgaccacgtcggacgtcgacgcggtcgaggcgcacggcaccggcaccacgctcggtgacccgatcgaggcccaggcgctgctggccacctacggccaggagcgggacgcggaccagccgctctggctcggctccgtcaagacgaacgtgggtcacacgcaggccgcggccggtgttgccggtgtgatcaagatggtgctcgcgatgcgggccggtgtgctgccgaagacgctgcatgtcaccgagccgtcgccgcacgtggactggtccgccggcgccgtggagctgctgacggagacgcgcgactggccggagaccggccgtccgcgccgcgccggtgtgtcgtcgttcggtgtgagcggcacgaacgcgcacgtcatcgtcgagcaggcacccgccgtcgaagaggagccgacgcgcgcccttccggcactcccggtcccgtggtccatctcggccaagtcggccgacgcactgcgcggccaggcccaggccctgtcgtcgttcgtctctgccgccggtgatgtgtcggtggtggatgtggcgtactcgctgggtgtgtcgcgtgcgggtcttgagcatcgtggtgtggtggtgggggagtcgcgtgcggagttgctcgtggctctggagtcgttggcgtccggggttgagtcgccgggtgtggtgacggggcgggtcgccgagggtcggttggcgttcctgttcacggggcagggtgcgcagcgggtcggcatgggtcgggaactcgctgccgaattcccggtgttcgcggcgtctcttgagcagacgtgtgacttgttggagcgtgccggggttgcggtgcgtgaggtgctgttcgctgaggagggttcggccgaggccgccctgctgacgcggacggtgtatgcgcaggctgcgttgttcgccgtcgaggtggcgctgttccggttggtggagtcgttcggtgtggtgccggactttgtcgccgggcattcggtgggtgagatcgccgccgcgcatgtcgcgggcgtgttctcgcttgaggatgcggtgtccctggtcgcggctcgtggtcggttgatggatgcgctgccggagggtggggcgatggtggcggtgcaggccaccgaggaggatgtgcttgccctgttggagggggtggaggacgcctccatcgccgcgatcaacggccccgacgccgtggtggtctccggcaccgagaccggtgtcgcccgcgtagtggacgtactccgggagcggggtgcgaagaccaagcggctcgtggtgagtcatgccttccactcgccgctgatggagccgatgctcgccgacttcgccaccgtcgtggaggggctctcgtacaaggctcccgccatcccggtcgtgtcgggatcagttgtcgggagcgagttgtcgacgccgggttactgggtgcggcatgttcgtgaggcggttcgtttcggtgcaggggtggagaccctgctcggcgctggtgtgtcgtcgttcctggagatcggtccggacggtgtgctgagcgggatggcccgtacgtccgtgcctgagggtgcggatgtggagtgcgcgccgctgatgcgccgggaccgcgccgaggtgcgggagttcctgaccggtctctcccgtctcgccgtacgcggtgtcccggtcgcctgggagtcgctggtcgaaggcggccggcgggtcgagctgccgacgtacgccttccagcgtcgccggttctggctggaggccggccgttcggtgacggatgcttcgggtctgggccagaccgccgcggagcaccccttggtgggtgccgtggtgggtctggccggtggcgatggtgtggtgctgaccggtcgggtgtcgttgcacacccacccctggttggcggatcaccaggtcgcgggggtgacgctgctgccgggtacgggctttgtggagctggcggtccgtgccggtgacgaggtcggctgtggccggctggaggaactgacgctggaggccccgctggtcgtgcccgaccggggcgcggtgcagctccaggtcgtcgtcggcgcactggagacgtccggtgtccgtaccgtcagcgtctactcccgcgtggaggaagagagcgcggatgacacctgggtccgccacgcgaccggcgcgctcatggccgccgccgaggagcccggattcgacctcaccgcatggccgccggctggtgcgcgggcggtcgatgtgagtgacgcttacgccggactggcgagccagggctacgggtacggacccgtcttccaggggctgacggccgcctggcgcaagggccgggaggtcttcgcggagatcacgctgccggaggccgcgcaggtggacgcttcggcgttcgggctgcaccccgcgctgctggacgctgcgctgcacgccaccgactacctcgatcaggagagcgccgagggcacgcatctgccgttcgcgtggagcggtgtctcgctgcacgccgccggcgcgtccgcgctgcgggtacggatcgtcgcgaccggcaaggacggatacgcgctcgacctcgccgacagcacaggccgccccgtggccactgttcagtcgctggtcacccgtcccgtcaccgcggaccaactgtccgaagcctcggccactcagcaccagtcgctcttccggatggagtggaccgccacgaccgccccggccgcgaccgctgatgtccgctgggcgcttctcggcgagccggtcgcggaactgggcgacctcgcgggcttcggccgtacgaccgcgtacgcctcgctcgccgacctggccgccgccgacgcggacaccgttccggacctcgtcgtcctgccgctgggcagccggatggctgagggaagtgacgtaccggaagcggtacgggagaacctcggtcaggtcctggcgacgatccagcagtggaacgacgactcccggttcgacggctcccggctcgtggtgctgacacacggtgccgtctcggtctccgacgatgaatcgctcaccgcactggatctggctccggtctggggtctggtccgctcggccatggccgagactcccggccgcttcctgctggtggacaccgatgggaccgacccgtcgaccgacgcgctgcgtcatctggccgaactccaggaggccgagttcgcgctgcgcgagggcgagatacgcgtacctcggctcgcgcgcgttccggtggccggagctggtgccgacatcacgggtggtttgggtgatggtgtggtgttggtgacgggtggtacgggtgggttgggtgctgtggtggcgcgtcatgcggtggtggtgcatggggtgcgtcgtttggtgttggtgtcgcgtcgtgggttgggtgctccgggtgcggtggggttggtggctgagcttgagagcttgggtgctgtggtggaggtggtggcgtgtgatgtgagtgatcgtgtggcgttggctggtgtggtggggggtattgggtcggatctgtcggctgtggtgcatacggcgggtgtggtggatgacggtgtggtgggttcgttgtcggtggggcggttgtcgtcggtgttgggtccgaaggcggatgcggcgtggtatttgcatgagttgacggtgaggttggatctgtcggcgtttgtgttgttctcgtcggttgctggtgtggtggatggtgcgggtcaggggaattatgcggcggcgaatgtgtttttggatgggttggctgtggtgcggcgtggtttggggttgccgggtacgtcggtggcgtgggggttgtgggagggggctggtatgggtgcggtgttgggtgaggctgatgtgttgcggatgagtcgttcgggtgttttggggttgtcggtgggtgaggggttggggttgtttgatgtggcgttgggtgaggatgtggcggcgttggtgccggtgcggttggatttgggggctttgcgttcgcgggttgatggggtgcctgcggtgttccggtcgttggtgcgggtgccggtgcgtcgttcggtgggtgtgggtgtttcgggtggtggtgaggtgtcgttggagcggcgtttggcggtgttggatgcgggtgagcgtgagcgggtgttgttggagttggtgcggtcgcatgtggcggctgtgttgggttatgagggtgcggggtcgattgagccgggtcgtgcgttcagtgatatcgggttcgattcgttgtcggcggttgagctgcggaatcgtttgaatggtgagacggggctgcggctgccggcgacgctgatcttcgattaccccaccccccaagccctcgccgatctcatccaggagaagacgctgggtctggtcgacgccgccgcggccaccgtgtccaccacggtgtcccgcaccgatgacgagcccatcgccatcgtcggcatgggctgccgctacccgggagacgtccgtacccccgaggagctgtggcggctcgtcgccagcggcacggacgccgtcagcctcttccccgaggaccggggctggaacgtcgactccatctacgacccgacgcccggcctgtccgggaagacgtacacccgtgagggcggcttcctgcacgacgcggccgagttcgacgccggcttcttcgggatctcgccgcgcgaggccctcgccatggacccgcagcagcgactgctccttgagacgtcctgggaggcgatggagcgggccggcatcgacccgaacagcctgcgcggcacccagaccggcgtcttcgccgggatcatgtaccacgactacgggtcccgggtgacccagccgtcggaggaggtcgagggctacctcggcaacggcagcgcgggcagcatcgcgtccggccgggtctcctacaccttcgggttcgagggtccggcggtcacggtcgacaccgcctgttcctcctcgctcgtcgccctccactgggcggctcaggccctgcgccagggcgagtgcagcatggcgctcgcgggcggtgtgaccgtcatgtcgacgccggagaccttcgtcgacttcagcctccagcgcggccttgccacgaacggccggtgcaaggcgttctcgtccgacgcggacggaactggctggggcgagggcgcgggaatgctcctgctggagcggttgtcggacgcgcggaagaacgggcacgaagtactcgcggtcatccgtggttcggcgctgaaccaggacggtgcgagcaatggtctgacggcgccgaacggtccttcgcagcagcgggtgatccgccaggcgctggccaacgccggtctgtcggccgccgaggtggacgcggtcgaggcgcacggtacgggtacgaagctgggtgacccgatcgaggcgcaggcgctcctggcgacctacggccaggagcgggacggcgaccagccgctgctgctgggctcgatcaagtcgaacgtcggccacacgcaggcggccgccggtgtcggcggtgtgatcaagatggtgatggccatcaggaacggtgtgctgccgaagacgctgcatgtcaccgagccgtcgccgcacgtggattggtccgctggtgcggtggagctgttggcggaggctcgggagtggcctgagacgggtcgtccgcgtcgtgcgggtgtgtcgtcgttcggtatcagtgggacgaatgcgcacgtgatcgtggagcaggcccccaccgaccagccggcgccgctgagcgagccggttggtgatgtgccgggtcttccggtgccgtggatcgtctcggccaagtccgcggaggcgttgcgggcgcaggcgggtcggttgggttcgttcttgggtgagaccggtgttgttgatgtgccggcggtgggttggtcgttggtgcggggtcgttcggtgttcgcgcatcgggcggttgtggtgggtggtgagtgggatgagctgttggccggtgtggctcgggtcgctgagggtgtggatgactcggttgtgtcgggtgtggcggatgtgtcggggcgtcgggtgtttgtgttcccggggcagggttcgcagtgggttggtatggcgcaggggttgttggattcgtcggtggtgttcacggagcggatgacggagtgtgctgcggcgttggatccgttggtggagtggtcgttgttggatgtggtgcggggtgtggagggtgcggcgtcgttggagcgggtggatgtggtgcagccggtgttgtgggcggtgatggtgtcgttggcgtcggtgtggcgttcggtgggtgtggtgccggatgcggtggtgggtcattcgcagggtgagatcgcggctgctgtggtgggtggttggttgtcgttggtggatggtgcgcgggtggtggcgttgcggtcgttggcgattcgtgaggtgttggcgggtggtggtggcatggtcgcggtccaggctgccgaggatgaggttgctgggtggcttgagggtgtggagggggtggggattgctgcggtgaatggtccgcgttcggtggtgatctcgggtacgcgtgcgggtttggatgcgtgtgtggagttgtggtctgggcgggggacgtgggtgaagcgggttccggtggactatgcctcgcattcggctgaggtggagcgggtgcgtgagcgggtgctggcggatctggcgagtgtgacgggtttgtcggggtcggtgccgatgttgtcgacgatgacgggtgactggattgttgagggtcaggtcggggccgggtattgggtggagaatctgcgtcgtccggtgttgttcgcggacgcgacgaggcggttggcgtcggagggcttcggtgcgttcgtcgaggtgagcgcgcatccggtcctggtgatgggcatcgaggagacgatcgaggcgctgaggtcgggcgccgccgatggtgcgaagtccggtgctggtgaggagagttcctccgctgtggtggcggtggggacgttgcgtcgtggtgagggtggttgggatcagttcctgcgttccctcgcggggctcttcgtccggggtgcggtggcaccggattgggagtcgttgttgggtggtgtgcgtcctcgggttgatttgccgacgtatgcgttccagcgtgagcggttgtggctggatgcgggtgtggtggcgggggatgtgtcggggttgggtcaggtggtggtgggtcatccgttgttgggtgctggtgtgggtgttgctggtgagggtgggggtgtgttgtttacgggtcgtttggggttgggttcgcatccgtggctgggtgatcatgcggtgtcgggtgtggtgttgttgccgggggctgcgtttgtggagttggtggtgcgtgcgggtgatgaggtggggtgtggtcggttggaggagttgacgttggcggctccgttggtggtgccggagcgtggttcggtgcggattcaggtggtggtgggtgctggtgatgggtcgggtgcgcgttcggtgggtgtgtggtcgtcggtgggggatgagggtgtgggtggggagtgggtgtgtcatgcgtcgggtttgttgacggctgatgtgggtgtggcgccggtgttgggtggtgtgtggccgccggtgggtggtgtggcggtggatgtgtcgggtgtgtatgaggggttggcgttggaggggtatgagtatgggtcggtgtttcgggggttgaggtcggtgtggcgtcgtggggatgaggtttttgctgaggtggcgttgggtgagggtgtgggggtggaggggtttggtttgcatccggcgttgttggatgcggcgttgcaggctgctgggtttggttcgtttgtgccggagtccgaggcagggtctgaggcgggttcgggtggggtgcggttgccgttctcgtggtcgggtgtgtcgttgtttgcgtcgggtgcttcggtggggcgggtgcggttgtggccggtgggtggggatggttttggtgtggagttgtttgatggggtggggatgccggtggcgcgggtggatgcgttggtgacgcgggagattagtgcgggtcagttgggtgcggctgctggtgccgggtcgttggtgggtggggagtcgttgttccgggtggagtgggctcctgtgtcgggtgtcgcgccggcttctgctggtgtgggtggttgtgtggtggtgggtgcggggagtgtgttgtctggttttggggaggtggttccggatctggcggcggtttccgcgggttctgcggatggggtgggtgtgccggggtgggtgttggtggatgtggatgcgtggttgggtgcggatctggctgtgggtgtggtgtcgggtgagggtgttccggtggtggcgcgtggtgtggtggcgcgggtgttggggttggtgcgggagtggttgggggatgagcggtgggtgtcgtcgcggttggtgtgggtgacgcgtggtgcggtgggtgctcgggtgttggatgaggtgtcgggtgtggtgtcgtcggggttgtgggggttggtgcgggctgctcagtcggagcatccggaccggttcgcgcttcttgatctggacagtgccaccgccgtggatgccgttcgtgatggtgtgttggggttgctggcggctggtgagccgcagttggtggtgcgtgagggtgaggtgctggccgcacggctgacccccgcccacaccaccgatgaacccaccggccaggagttcggcaaggcggcgaccggtacggtcctcgtcaccggtggcaccggcggcctcggtgccgtcgtcgcacggcacctggtcaccgagcacggcgcacagcgtctgctcctcaccagccgtcgcggtatcaacgcgccgggagccgccgaactcgtcgcggaactcgccgagctgggtgccagggccgacgtcgcagcctgcgacgtctcagaccgggcggcactcaaggaactgctcgccggcgtaccgggggacgcaccgctcaccgcggtcgtccacgcggccggcgtgctggacgatggtgtgatcgagtcgatgacggccgatcgcctggacgccgtcatgcggccgaaggtcgacgcggcctggcatctgcacgagctgaccgccgaccgggaattggatgcgttcgtcctcttctcctccgcggccggcacgctggacggtgccggacagtccaactacgcggcggcgaatgtgttcctggacgcgctcgcgcagtaccgccgtggtcagggtctggcgggtctgtcgctggcctggggcctgtggggcgagagcaccggcatggtgggcgcgctggagggttcggacctcgaccgcatcggccgctccggcgtccgggcgctgtcgtcggccgaggggctcgcgctcttcgacgccgccgccgtgctgggcgagccggccgtactgccggtcgcgttggacctcggcgtactccggacacagccccggaaccaggttccggcgatcctgcgcgggttcgcggcgggcccgacccggcgcaccgcggtcactggtgggcccgagacggaccaggaggcgctcacgcggcgcctcgcttccctctcgcccgccgaccgggaccgcttcctgttggatctggtccgtacgcaggtggccggggtgctcgggtactccggtccggacgcgatcgatccgcagcgcgggttccaggagctgggcgtcgactcgctggccgccgtacagatccgtaaccggctcggcgcggccacgggtgtccggccgccgacgacgctcgtcttcgactatccgacccccgacgcggtggccgggtacttcaaggagcacctcgtcatcgaggaggaggactcgaccgcggagctgatgcgggagatcgcccggctggaggccgcggtcacctccgcggcctcatcggcgggcggcgcgggcctggcacccgccgtggaccggctgcgggcgatggcggcgaagctggccgacgcggatgcccagcgggccgacgaggacgacccgggcctggagtccgcgaccgcggacgaactgttcgacatcctcgacggtgagctgtccacggactga

SEQ ID NO: 2 - protein, 355 aa, Streptomyces halstedii sp.HC-34
VKALVLSGGSGTRLRPITHTSAKQLVPVANKPILFYVLESIAEAGITDVGIVVGHTAPEVQDAVGDGSAFGVDVTYIAQDEPLGLAHAVRISRDYLGDDDFVMFLGDNFIIDGVTGLVDRFRDERPDAQILLTRVPDPRAFGVAVLDEQGRVIGLEEKPEHPRSDLALAGVYFFTPLIHEAVWAVKPSWRGELEITDSIQHLIDTGADVRSHIIDGYWKDTGNVVDILEVNRIILESVETAIDGDVDADSEIIGRVVIEKGATVKGSRIVGPALIATGTEIRDSYVGPFTSVAENCRIHDSELEFSIMMRDSSLTGIRRIEASLIGRHVHGTAVPRVPNAHRLVLGDHSTFQISS

SEQ ID NO: 3 - protein, 323 aa, Streptomyces halstedii sp.HC-34
MSTRLLVTGAAGFIGSAYVRGLLTDQPDLRVTVLDSLTYAGNRANLDLTHPSLDLVEGDICDTELVDRLTAEADQIVHFAAESHVDRSITGSAEFIRTNVLGTHTLLDAALRHGIDRFVHISTDEVYGSIEKGSWPETDPLRPNSPYSASKASSDLLALAYHRTHGLDVRVTRCSNNYGPYQHPEKVIPLFVTNLLDGKRVPLYGDGQNVRDWLHVEDHCAAIECVRTRGGAGEIYNIGGGTELSNRELTGLLLEACGADWDSVEYVTDRKGHDLRYSVDWSKVADLGYTPAHDFRAGLAETVDWYRSNRTWWEPAKYGHSPA

SEQ ID NO: 4 - protein, 419 aa, Streptomyces halstedii sp.HC-34
MRVLMTVFANRSHLYNMVPLAWALTTAGHEVHIASHPDNVQAISDSGLTAVPVGNDLNIMALAQSTPREEMVNGGALTLNETRPEKLTWQYIHDVFAQYSQIYEYMADSTMTADLVAHARQWQPDLVIWDALTYAGPIAAEAVGAPHVRMLFGLDQWGRMRDHFNRLTGERAADDRHDPLADWLATKGEPHGVAFTESLVTGTTTLAVAPPWMSFPSEQPALSMRHLPFNGPAVLPDWLREAPSRPRVCLTLGLTLRELADDNVTLADFVNAVADIDADVVATFSAEQVAEIGDLPDNVRAVDFVPLHALLPSCAAIVHHGGGGTRTNAIRYGVPQLIVPNWLWDEGYVAERFAERGAALVTEVPDLTPDRLRDQLRRLIAEPSFKAAAEQIQKEYDALPSLTETVGELVRVAERGRSL

SEQ ID NO: 5 - protein, 2260 aa, Streptomyces halstedii sp.HC-34
VENEKKLLDYLKRATTDLREARRRLREMEEKDQEPIAIVGIGCRFPRGVESAEQLWDLVADGGEALTPFPEDRGWDTDSLYHPDPEHLGTSYTNVGAFLHDAAEFDPGFFGISPREALAMDPQQRLLLETSWEAMERAGIDPAGLRGSRTGVFTGLMYFDYGSRVHSAPEDIEGYLGNGSAGSIASGRVAYTFGFEGPAVTLDTACSSSLVAIHLAAQSLRKGECTLALAGGASVMSTPDIFVDFSRQRGLSADGRCKAFSSDADGTGWGEGVGVLLLERLSDARKNGHRILGLVRGSAVNQDGASSGLTAPNGPSQQRVIRQALANAGLSAAEVDAVEAHGTGTKLGDPIEAQALLATYGQEREEGRPLWLGSIKSNVGHTQAAAGVAGVIKMVLAMRAGVLPKTLHVSEPSPHVDWSAGAVELLTETRDWPETGRPRRAGVSSFGISGTNAHVIVEQAPMDEAMDEVAGERETPAAGSDRTVPWVISAKSADALRAQAGRLSTFLAGTEGTLTTGISSETSVAAVSADASAVGWSLVRGRSVFAHRAVVVGGEWDALLASIGELADGAEDGSGAASGSVVSGVADVSGRRVFVFPGQGSQWVGMAQGLLDSSVMFTERMTECAAALDPLVEWSLLDVVRGVEGAASLERVDVVQPVLWAVMVSLASVWRSVGVVPDAVVGHSQGEIAAAVVGGWLSLVDGARVVALRSLAIREVLAGGGGMVAVQAAEDEVAGWLEGVEGVGIAAVNGPRSVVISGTRAGLDACVELWSGRGTWVKRVPVDYASHSAEVERVRERVLADLASVTGLSGSVPMLSTMTGDWIVEGQVGAGYWVENLRRPVLFADATRRLASEGFGAFVEVSAHPVLVMGIEETIEALRSGAADGAKSGAGEESSSAVVAVGTLRRGEGGWDQFLRSLAGLFVRGAVTPDWESLLGGVRPRVDLPTYAFQRERLWLDAGVVAGDVSGLGQVVVGHPLLGAGVGVAGEGGGVLFTGRLGLGSHPWLGDHAVSGVVLLPGAAFVELVVRAGDEVGCGRLEELTLAAPLVVPERGSVRIQVVVGAGDGSGARSVGVWSSVGDEGVGGEWVCHASGLLTADVGVAPVLGGVWPPVGGVAVDVSGVYEGLALEGYEYGSVFRGLRSVWRRGDEVFAEVALGEGVGVEGFGLHPALLDAALQAAGFGSFVPESEAGSEAGSGGVRLPFSWSGVSLFASGASVGRVRLWPVGGDGFGVELFDGVGMPVARVDALVTREISAGQLGAAAGAGSLVGGESLFRVEWAPVSGVAPASAGVGGCVVVGAGSVLSGFGEVVPDLAAVSAGSAAVPGWVLVDVDAWLGADLAVGVVSGEGVPVVARGVVARVLGLVREWLGDERWVSSRLVWVTRGAVGARVLDEVSGVVSSGLWGLVRAAQSEHPDRFALLDLDSATAVDAVRDGVLGLLAAGEPQLVVREGEVLAARLTPAHTTDALIPLADHAPWRLAKDPGGSLDALTVVPAPDVLEPLTEGQVRIAVRAAGVNFRDVLMALGMVPARGTQLGGEAAGVVTAVGPGVTGIAVGDRVMGVFDGPFGPVAVADRRMVSRIPDAWSYTEAATIPLVYLTAYYGLVDLAALQQGQRILVHAATGGVGMAAVQLARHFGAEVYGTASPGKWDTLRAMGFDEAHMASSRSLDFEDHFWQTTGGEGFDVVLNSLAQEYVDASLRLQPRGGRFLEMGKTDIRDADEVAAAHEGVRYEAYDLTVFTSVDGPGAIPERIQEMLSELLALFDKGVLTPLPVTTWDVRRASAALRHMSQARHTGKIALTVPRPLDQDGTVLVTGGTGVLGSLLARHLVTEHGVRNLLLVSRRGGDAPGAAELVAELTAAGAEVSVVACDTADRAALEKLLASVPGDAPLTGVFHTAGVLDDGIVEAMTPERVDAVMRPKVDAAWHLHELTEGLDLAAFVLYSSAAGVSGDAGQSNYAAANVFLDALAQRRRAAGLPGQSLAWGLWDDRSEMTGHLGDAEIARMTEAGVLGFTAADGLAALDRAAMYDDAVLVPMKLDTATLGAGSSPVPHLFRGLVRTPVVRRAVAGNTGGDSGGGLEQRLAALTAAERTETVLELVRERVAAVLGHASADAIDPARAFKEIGFDSLTAVELRNRLNAATGLRLPATLVFDYPTPTVLSQYLLAELAPGLPAEDPVGTRLLEQIARIEAVLSEVSEVTDETSSLSDMDADARSGITARLNDILTAWNRAQRAPGHDAVAAELDDASDDEIFDFIDSTFGKS

SEQ ID NO: 6 - protein, 3362 aa, Streptomyces halstedii sp.HC-34
MANEAKLREYLKRVTTDLHETNERLREVEGRANEPIAIVGMSCRFPGGVESPEQLWELFRTGTDAIGEFPEGRGWDVEGLYHPDPDHAGTSYTREGGFVHGAERFDPSLFGISPREAVSMDPQQRLLLETSWEALEASGLDPLRLKGSRTGVFVGVMSSDYGIQKGSAPDGVEGFLSTGTHSSIVSGRVSYVLGLEGPAVSVDTACSSSLVALHSAAHALRQGECSLALAGGVTIMSTPERFVEFSRQRALSADGRCKAFSASADGTGWSEGVGMLVLERLSDARKNGHRVLAVIRGSALNQDGASNGLTAPNGPSQQRVIRQALASAGLTAAEVDAVEAHGTGTTLGDPIEAQALLATYGKEREDGRPLLLGSSKSNLGHTQAAAGVAGVIKMVLAMRAGVLPKTLHVSEPSPHVDWSAGAVELLTEAREWPETGRPRRAGVSSFGFSGTNAHVIIEEASEFEPSAVEPLAGSGVTPPWVLSARSADALRGQAERLLSFVSAAGDVSVVDVAYSLGVSRAGLEHRGVVVGESRAELLAALESLASGVESPGVVTGRVAEGRLAFLFTGQGAQRVGMGRELAAAFPLFAASLEETCGLLERAGVAVREVLFAEEGSAEAALLTRTVYAQAALFAVEVALFRLVESFGVVPDFVAGHSVGEIAAAHVAGVFSLEDAVSLVAARGRLMDALPEGGAMVAVQATEEDVLALLEGVEDASIAAINGPDAVVVSGTEAGVARVVDVLRERGAKTKRLVVSHAFHSPLMEPMLAEFATVVEGLSFAAPTIPVVSNVSGAVADAELSSPGYWVRHVREAVRFGAGVETLLGAGVSSFLEIGPDGVLSGMARTSVPEGADVECAPLMRRGRGEVREFLTGLSRMVVRGVPVDWQSLVEGGRLAGLPTYAFQRERFWLDVPSAVGDVATAGLAPSGHPLLGAVMRRADVDGVVFTGRWSLRSHPWLGEHRVGSSVVFPGTGFVELLMRAGDEVGCGRIEELNQETPLVVPERGALQLQVVVGAPEETGLRGVGVYSRAEDADADVPWTRHASGLLSPAVVPADFELTQWPPAGAEALNVEDTYQRLADAGLVYGERFQGLKSGWIKGEDIYAEIALPEHAVAEAAEYTLHPAALDAALQASGLNDPPEVQATAYVPFSWSGVSLFASGASVLRVCVRHVARDRVSLLVADGVGVPVAVVESLVLRAISAGAVAVAGVGSGVGGGLFEVVWSPVVGVRGVDVSGVVVLEAGVGVGGDGVSVVGGVLEGLQGVLGGGSGSRVVVVTRGAVGSGGVVDVSGAGVWGLVRSVQAEHPGRLVLVDVGVEGDVGVGVGLALGSGEEQVVVRGGEVFVPRLARVGAVAADAGVDGAEIAGGLGDGVVLVTGGTGGLGALVARHVVVERGVRRLVLVSRRGLGAPGAVGLVAELEGLGAVVEVVACDVSDRVALAGVVGGIGSDLSAVVHTAGVVDDGVVESMSVGRVASVFGPKADAAWFLHELTRDMGLSAFVLFSSMAGTVGGGGQSNYAAANAYLDGLAEYRRGLGLAATSLAWGLWEESTGMGSRLTDADLDRMSRSGIRTLSIEDGLALFDAALAADRPTVMPAHFDIPALRGQGESLAPVFRTLAGPPARRSAAVTPRDIAAATESSLTDRLAGLDAEGRRALVLGVVRAQVAQVLAYASPDLVEPERAFQDLGFDSLTAVELRNGLTAIAGVRLPATLVFDYPSTDILTDFLLAELSDEIPAAVATLPAMGHVVDDDPIAVIGMGCRYPGGVESPEELWKLMAEGRDAISEFPTDRGWDLDAIYHPDPMHTGTSYTREGGFIHNAGDFDAAFFGISPREAMETDPQQRLLLETSWEAFEQAGIVPTDLKGTQTGVFAGVMYHDYAGNIGSGSIVTGRVAYTLGLEGPAVSIDTACSSSLVAIHLAAQSLRQGECSMAIAGGVAVMATPESFIEFSRQRALSQNGRCRAFSSDADGTAWGEGVGVLILERLSDARKNGHEVLAVIRGSALNQDGASNGLTAPNGPSQQRVIRQALANSGLSAAEVDAVEAHGTGTTLGDPIEAQALLATYGKERDADQPLWLGSSKSNFGHTKAAAGVAGVIKMVMAIRNGVLPKTLHVTEPSPHVDWSAGAVELLAEAREWPETGRPRRAGVSSFGISGTNAHVIVEQAPTDQAATKPKAADAVPGLPVPWVVSAKNPEALRAQAGRLGSFLGETGVVDVPAVGWSLVRGRSVFAHRAVVVGGEWDALLASIGELADGAEDGSGAASGSVVSGVADVSGRRVFVFPGQGSQWVGMAQGLLDSSVVFTERMTECAAALDPLVEWSLLDVVRGVEGAASLERVDVVQPVLWAVMVSLASVWRSVGVVPDAVVGHSQGEIAAAVVGGWLSLVDGARVVALRSLAIREVLAGGGGMVAVQAAEDEVAGWLEGVEGVGIAAVNGPRSVVISGTRAGLDACVELWSGRGTWVKRVPVDYASHSAEVERVRERVLADLASVTGLSGSVPMLSTMTGDWIVEGQVGAGYWVENLRRPVLFADATRRLASEGFGAFVEVSAHPVLVMGIEETLEAHHTATTDDDTTTRTPVVTVGTLRRGEGGWDQFLRSLAGLFVRGAVTPDWESLLGGTRPRVDLPTYAFQRRRFWIESVRKEAVTLAADPVDAAFWEAVESADLAKLADSLRIETDVLEGVLPALTSWRTRSREQSLVDGWRYREEWKPVPAPTPNSATGTWTVLVPATHQGDATVTGVLDGLRRGGADIRVLEVTATDREALAEQLRAELAQSEPRLILSLLALDDRAHPRHPELTEGITATIALVQALDDCGATARLWCATSLAVAVTESAEVLNPVQTTAWGMGASFALDHPETWGGLVDLPADIDTRTADILCSVLASDLQEDQIALRTAGLFARRMVRARLDESAVATEQPWRPNGTVLVTGGTGGIGSHVARWLAAAGAEHLVLTSRRGAEAPGAAELEAELALLGAKVTLAACDMGDRESVRELVAALPDAAPLTAVFHLAGALPDGERRLSATTFEDFSRMTRAKIGGAVHLDELLADRELAAFVTFSSGSAIWGNANQAAYGASNAFLDGLVHNRRARGLAGTSIAWGLWGGDGTDTETDEQLSRIGVRSMDPRLALEVLRQALDQDTSHLIATDIDWQRFAPVFTIARPRPLLDGIAEVRAVLSADTAEVGPADDDTPKETVVTRLAGLSPAERDHALLELVRTQVAAVLRYSDTSDVEQDQSFKDLGFDSVTAVELRNKLTRGVRAAAARHGVFDYATPVALAAHLRSELFTDDAAASGEVPLLAELDRIEAAVTSLPSADIERMHLTSRLQSLVTKLNDIVGAGAPLEAEAIADKLETATADDIFAFIDKDLGLN

SEQ ID NO: 7 - protein, 3808 aa, Streptomyces halstedii sp.HC-34
MSNEEKLLDYLKRVTADLHATRQRLREAESDEQEPIAVVAMGCRYPGDVRTPEDLWQLVATGGDAVTEFPADRGWDFDTLLGGDAGASGSTYVARGGFVHDAADFDADFFGISPREALAMDPQQRLLLELAWETSERAGIDPHSLRGASVGVFAGTNGQDYADLMDRAPEDTEAYLSTGSVAAVVSGRVSYALGLEGPSVSVDTACSSSLVALHLACQALRQKECSMAFAGGVTIMSTPGPFIAFSRQSGLASDGRCKAFSDDTDGTGWGEGAGLLLLERLSDARRNGHQVLAVVRGSALNQDGASNGLTAPNGPSQQRVIRQALANAGLTAAEVDAVEAHGTGTTLGDPIEAQAILATYGQSREQDQPLWLGSIKSNIGHTQAAAGVSGVIKMVMAIQNGVLPKTLHVSEPSSVVDWSAGAVELLTEAREWPETGRPRRAGVSSFGVSGTNAHIIMEQAPVPEAESEPDGEAPAAVSGLPVPWVVSGKTADALRAQAERLLSFVSADADVSVVDVAYSLGVSRAGLEHRGVVVGESRAELLAALESLASGVESPGVVTGRVAEGRLAFLFTGQGAQRVGMGRELAAAFPVFAASLEETCGLLERAGVAVREVLFAEEGSAEAALLTRTVYAQAALFAVEVALFRLVESFGVVPDFVAGHSVGEIAAAHVAGVFSLEDAVSLVAARGRLMDALPEGGAMVAVQATEEDVLALLEGVEDASIAAINGPDAVVVSGTEAGVARVVDVLRERGAKTKRLDVSHAFHSPLMEPMLAEFATVVEGLSFAAPTIPVVSNVSGAVADAELSSPGYWVRHVREAVRFGAGVETLLGAGVSSFLEIGPDGVLSGMARGSISDGADVGCVPVMRRGRGEVREFLTGLSRIAVRGVPVSWGPLLAGGRRVELPTYAFQRRRFWLEAGHSVTDASGLGQTAAGHPLIGAVVSLAGGDGAVLTGRVSLNTHPWLGDHRVASSVVFPGTGFVELLMRAGDEVGCGRLEELNQEAPLVVPDRGAVQIQVTVEAPNAVGERPVAVYSRLEDTDTDAPWTRHASGLLSPTTASADFDFTAWPPAGADPLSVDGMYDRPDSGLVYGPLFQGLTAAWRKGDEVYAEIELPEGAAVDAARFGMHPALLDAALHALGFSASYEDQEEDGTVARLPFSWSGVSLFASGASVLRVCVRHVARDRVSLLVADGVGVPVAVVESLVLRAISAGAVAVAGVGSGVGGGLFEVVWSPVVGVRGVDVSGVVVLEAGVGVGGDGVSVVGGVLEGLQGVLGGGSGSRVVVVTRGAVGSGGVVDVSGAGVWGLVRSVQAEHPGRLVLVDVGVEGDVGVGVGLALGSGEEQVVVRGGEVFVPRLARVSVVAADVVPDAGVDGADIRGGLGDGVVLVTGGTGGLGALVARHVVVERGVRRLVLVSRRGLGAPGAVGLVAELESLGAVVEVVACDVSDRVALAGVVGGIGSDLSAVVHTAGVVDDGVVESMSVGRVASVFGPKADAAWFLHELTRDMGLSAFVLFSSMAGTVGGGGQSNYAAANAYLDGLAEYRRGLGLAATSLAWGLWEDTANGGMTGHLAEADRTRMARGGVFPLALDEGLALFDAALATGKATLVPVHLDTTALRAHADELPALFRDLVRAPKRRTAAEGRAADSADDLAGRLAGLAAEARRPLVLGVVRAQVAQVLGYASADLVEPERAFQDLGFDSLTAVELRNGLTAVAGVRLPATLVFDYPSTDILTDFLLAELSGKVAVAAPLAPLATTGPVHDDPIVVIGMGCRYPGGVRSPEDLWRLVADGRDAISEFPTDRGWDLDALYHPDPDHAGTSYTREGGFLHDAADFDADFFGISPREAIVMDPQQRLLLETSWEAFEQAGIVPADLKGTQTGVFAGVMYHDYGTRIVDIPEGAEGYLGTGISGSVVSGRVAYTLGLEGPAVTIDTACSSSLVALHSAAHALRQGECSMAIAGGVTVMAGPDTFIDFSRQRGLATNGRCKAFSADADGTGWGEGVGVLVLERLSDARKNGHEVLAVIRGSALNQDGASNGLTAPNGPSQQRVIRQALASAGLTTSDVDAVEAHGTGTTLGDPIEAQAVLATYGQDRAADQPLWLGSAKSNFGHTQAAAGVAGVIKMVMAIRNGVLPKTLHVSEPSTHVDWSAGAVELLAEAREWPETGRPRRAGVSSFGVSGTNAHVIVEQAPTEEQAPADAPAPTDAPAGTPVPWIVSGRTADSLRDQARRLLEHLDRNGDLDPQDVARALLTTRTRFHHRAAVVATERQDIVAALEALADGRPVSGLVQGTATTMAKSAFLFTGQGAQRVGMGRELAAEFPVFAASLEQTCGLLERAGVAVREVLFAEEGSAEAALLTRTVYAQAALFAVEVALFRLVESFGVVPDFVAGHSVGEIAAAHVAGVFSLEDAVSLVAARGRLMDALPEGGAMVAVQATEEDVLALLEGVEDASIAAINGPDAVVVSGTETGVARVVDVLRERGAKTKRLDVSHAFHSPLMEPMLADFARVVAGLSYEDPAIPVVSNVSGSMADGELSTPGYWVRHVREAVRFGAGVETLLGAGVSSFLEIGPDGVLSGMARTSVPEGADVECAPLMRRDRAEVREFLTGLSRLAVRGVPVSWSPLVAGGRRVELPTYAFQRRRFWLDAGHSVTDASGLGQTAAGHPLIGAVVGLAGGDGVVLTGRVSLHTHPWLADHQVAGVTLLPGTGFVELAVRAGDEVGCGRLEELTLEAPLVVPDRGAVQLQVVVGGLEESGVRTVSVYSRAEDTDNPDTPWTRHASGALGTAADPADFDLTAWPPAGTEAVEIGDFYGELAATPDGLVYGPLFQGLTAAWRKGDEVYAEIELPEGAAVDAARFGMHPALLDAALHAVGLSNDADADADAGTAQEDGTVARLPFAWSGVTLHRSGATRLRLSVRPTGGDSYHLRIADAVGAPVATVGELLLRGISAEQLARAGTDRPDALFRLEWEALRGDGATTAGEWALLGGDPYGLAPARAVPYGDLDALAAEAEGGTPPEVVLLPLTVPAGEEPDAGERAAHRALRAVRSWLADDRFAAATLVLMTRGALAATPGDEVTDVAGAEVWGLVRSAQTEHPGRFVLVDSDGSEASARALPAAVASGEPQLALRAGTVNAARLGRVERAGAEAVATFDPERTVLITGGTGALAGQLARHLARAYGVRHLLLAGRRGPSAPHAAELVAELAELGALAEVVACDVADREALTALLAAVPADRPLGAVVHTAGVLDDGLVESLTPERLDAVLHPKATAALLLDELTRDADLTAFVLFSSAAGTLGSPGQANYAAANAGLDALAVRRRGQGLPGLSLGWGLWQADGGMGGALSGGDQARIARGGVAALTTDHGLALFDTACAGPDAVVLPMLLDLRPQDDVPHLLRSLVSARRKGASAGARHEGPAELRRRLAAATPDERYEHLLGLVRSCTAVVLGHRGPQDVDPGVGFLESGFDSLTAMELRNQLNEATGLRLAATVVFDHTTPADLARHLVDGVADTVAAAPGQQPAPRSDDDADTLSGLFRTAVRAGQVTKGVDLLRAVAELRPAFHSADELGDDLPVPVRLAQGPSAARLFCFASPMAMGGTHQYARLAAHFRGVREVSALPMPGFATGDLLPATAEAVVEVFGRSLLRAAGDEPFVLLGYSAGGVFAHAVAAWLESEGCAPAAVVLLDSYRADGGTSMDGDFWVSMVEGLFSREEVFGRFTSARLSAMGRYARLVGEVKTGEVKAPVLFVRPERSLSASADAGGVGGGDAGAAAATDDWRASWDTAETVLEVAGDHFDIIESQAAATADAVEGWLADRV

SEQ ID NO: 8 - protein, 236 aa, Streptomyces halstedii sp.HC-34
MYEEDFARVYDDIYRRHKDYAGEAARIRELALERNPGAADLLDVGCGTGEHLALLRDDFAVTGVDLSAPMVRVAEAKLPGVPVHEGDMRTFGLDRSFDVICSMYSSVGYLETLDGLFAALKNMAHHLRPGGVLIIEPWILRDSWNGGDLVEASFENEGGKVVRMGRWTTRAGRSHVEMHYLVTSGDDPVRHFVDEQELSLFSKDEYEEAFRAAGCSVEYLPDGYVDRGVFVGVRQP

SEQ ID NO: 9 - protein, 397 aa, Streptomyces halstedii sp.HC-34
LRLGAGERNDEQVTDSPVFREPLHVGRPNIGSRARLTERLEGALDRLYLTNGPLVREFEERLAEVAGVDHCVAVCNATIGLQVAAKAAGIRGGDEVIVPAFTWVATAAALDWIGIVPVFCDADETTGNIDWTKVEALIGPRTRGIMGVHVFGRPCEVDELEEIAGRHGLPLLFDSAHAIGCTYKGRPVGGFGTAEVYSFHATKFVNSFEGGAIVTRDAEFAERCRELRNFGITSAGEIRSGGTNAKMNEAAAAMGLTSLEVMDSLMEANRINHGRYEKGLDGLPGVWVRGQAEGERANHQYLVIEIDAAVAGIHRDEVHTALIAQNVLSRRYFHPSCHQVEPYLSDPARHAPHPLPHAEALAERVLALPTGTTVGPSEIDQVCDIIRRCVSARTPAR

SEQ ID NO: 10 - protein, 493 aa, Streptomyces halstedii sp.HC-34
MEISMTETLSTLPPTHTGDESLAVRIAKSVSVVDDGALHSLDEFHDWFTARGQRTAHVERVPLDELTGWSSDPVTGNIGHDSGKFFSVQGLSVELPGAPVPAWAQPIINQPEIGILGILIKEFNGVLHCLMQAKLEPGNRNGLQLSPTVQATRSNYTRVHQGNPVPYVGYFQDTSANRVITDALQSEQGSWFYQKRNRNMVVETEAEIEQHEAFMWLTIGQLHRLLAIDDLINMDTRTVLSCLPFSGAQLAEQLPGTGGDLRMPILRSCSEDQGSLHTTGDLLSWITDARTRNEVRTRLTPLRDVAGWRRTPDAITHDTGRFFDVMAVRIETDGDREVRQWTQPMIAPAGIGIVAFLVKRIDGVLHVLAHARVEPGYLDVVELSPTVMCTPGNYEGLPAAARPPFLGEVLSARPEQVRFETILSEEGGRFYHAQNRYIIVESDIDVAPELASEYRWMALHQMVGLLRHSHYVNVQARSLIACLHSLSGAQGRN

SEQ ID NO: 11 - protein, 330 aa, Streptomyces halstedii sp.HC-34
VTMLYRQLGRTALKVSPLCLGTLNLGVRTTRDEAFALMDEALEQGINFFDTANQYGWQVHKGLTEEIIGEWFAQGGGRRERVVLGTKVCNPMSDLPNDQGLSARHIIASCEDSLRRLRTDWIDVYQLHNIDPTASWDEVWQAMETLVHQGKIRYVGSSNFAGWHLADAQAAAARRNFLGLVSEQCCYNLATRYVEMEVVPAARAHGIGVLAWSPLHGGLLSGALRKLDEGTAVKTAQGRAQIALRTMRDTVEQYEKLCESISADPSQVALAWLLSRPGLTAAVIGPRTTGHLASALETLDMTLPEDILAPLDELFPPIGNGQPAPATWLA

SEQ ID NO: 12 - protein, 158 aa, Streptomyces halstedii sp.HC-34
MPDITSERLPDQDHHLPAERARPVLISSVASDSHTWNLVFLQLLIEELGHEVINLGPCVPDELLIAECRDRRPGLVVISTVNGHGYQDGLRVIGKLRACEDLANIPVVIGGKLGVSGPGQSYAAELAGAGFDAVFPDGAEAVSSFTRYVEKLPQRVVS

SEQ ID NO: 13 - protein, 469 aa, Streptomyces halstedii sp.HC-34
LTGFGAYVRSVHRRGELVVQPRMGFSGTVEMRAGLAATRAADAATVGTITLDSYTRVGELAAAEEALRSGIALNGYPIVTYDESTTRELLAGIAGEDFPVQIRHGSATPLHIFGALTRAGLDATEGGPVSYCLPYGRIPLEQSVTNWTRCCEEFARLRGLGREPHLETFGGCMLGQLCPPSQLVAISMLEALFFHQHGIRSISLSYAQQTHQGQDEEAVLALRRLHRELLPDADCHIVIYAYMGVYPTTPEGAHALLGRAAELAVATGAERLIVKTAAEAHRIPTIGENVAALEHAATVSRAARRATALPAGHSAAPAATDPTAALTARPGTYADGSAALTAGPGAARDGSTDSAVYAEARALIEAVMNCGPDVGTALFRAFKRGYLDIPYCLHPDNMGRSRSYIDDTGRLQWAETGMLPLGNARKSGTGRSVSSTDLISALSFVQRTYDQLAFPESREPLRIPRQQRP

SEQ ID NO: 14 - protein, 299 aa, Streptomyces halstedii sp.HC-34
MSKPPSAKGTVPFGQYRTWYRVTGDLHSGKPPVVLLHGGPGSTHDYLLAMTSLTEAGWPVVHYDQLGNGGSTHLPEKGEDFWTVQLFEDELDNLLNQLGIAGDYVLFGQSWGGMLGSVHAARRPAGLRGLVVANAPASMKIWLQEMARLRALLPPDVQETLLKHEAARTTDTEEYFHAMRAFYDRHVCRIVPWPRDFAATFMEIYNDPTVYTTMNGPNEFHVIGTLRDWSVEDCLPDIQVPTMVLIGRHDEATPATVKPFLDLVPDVRYEVLENSSHVPHLEEPERFHEVMIDYLESLV

SEQ ID NO: 15 - protein, 327 aa, Streptomyces halstedii sp.HC-34
VNATVETTQHDVEGTGAAGATAMLFPGMGPAAFSDVGRFMVTNRYTRELLAEADDTLGYSLVDRFRQAEGDYSEYAQIAFLVNCVALARWAEQTMDLTPRICAGASFGEKSVAAYSGALTFADAVRMTAGLARCMDEYFRTEHLGVVTHSFVRAPRERLDEILAELDERGEWHEISCHIDHDFFMLTLHERNSVWLEGRLRSVGAMPLYAMRPPMHAAAFGGLRDKAEEEVIAPLTFHDPTLPVVADQDGKVLTTGDEVRTMLLESFVRPLRWPDVISSLQDQGVTRVCVAGPDSLFGRVGTTTRAFEVIAATPRLALQPRARTTSR

SEQ ID NO: 16 - protein, 82 aa, Streptomyces halstedii sp.HC-34
MWDAQFENLLRRYLPFLSADQPLEQDINLRDIGLDSLGTVELLSELENTYDVHFQDEALTKETFETPGVLWKTLSQMVEPRH

SEQ ID NO: 17 - protein, 524 aa, Streptomyces halstedii sp.HC-34
MHAADHALHARFLRGLACAPDRPAVRFGGRTLTYAQAHRTALTWAGSLLRATPEPPAAVGVLADKGIPAYLGILTALYAGAAVVPLRPDFPAARTAEMMRAAGVTAVIADGRGRRLLPELLADRRDTAVLAADEEGAPADESPADGSAPGRRVAIDEGYALTAPRDVVPDDTAYVLFTSGSTGRPKGVPLSHGNIAHYFEVLDARYDFTADDVFTQTFDLNFDCSLFDLFCAWGAGASVIQIPPQAYRDLPSHLAEQGVTVWFSTPSSIALVRRLGGLAPGSLPTLRWSFFAGEALKCADTEDWQRAAPASFVENLYGPTELTVTVTAHRWSPEVSPVVGANGVVPIGPLHKGLDHVLIDAGGLPHPDTGELCVTGPQMAGRYLDPADDHGRFLDHDGRRWYRTGDRVRLAPGGELVYLGRMDAQVQIQGWRVELAEVDHALQGCEGVGEAVTVGAATDAGTELVVFYTAPAPVPPVRFAAVLRATLPDGVVPRHYRHVAELPLNSNRKIDRRALTARAEELLG

SEQ ID NO: 18 - protein, 478 aa, Streptomyces halstedii sp.HC-34
MEDAFGTWTYQELLNHSQAFSAWLDGKGVARGERIVVQLPNIRQTVAVFYGACRRGVVFVPLNPGMKPFHLRSVIADADPRLVIAEDETAADRLRDVTDLPVYSIDSLWADVERLRDAGAGAEAVEVSPEDLAVLIYTSGSTAAPKAVACPHQQIVFAASSINAVLGYHAEDIVFCRMSVSWDFGLYKVLISTLTGAKLVLAGGEPDIALVKSLRESGATMMPIVPSLASMLTTLIRRDPEGAPTLRMFTNSAAALPQVTIDALRSAFPGAQVVRMYGQTECKRISIMPPHLEHERPDSVGLPLPGTTIEILDEDGTLLPPGEPGEITVTGPHVMAGYWRAPEITARAYRRDETTGAMRLHTGDYGHLDEDGFLYFGGRRDDMFKRKGTRMSTVEIEAAALDIPGVTAAVALPPTATRDLALCVASDLEPHDVLRSLAERLEPAKVPATCRIVNDFPLTLNGKSERKQLARLLDGSDK

SEQ ID NO: 19 - protein, 414 aa, Streptomyces halstedii sp.HC-34
VNQYEELADQYGTPLYVYDLDRVAEARHDLRTSLPDEVEIYYALKANPHPQVAGALRSGEGRECRAEISSVGELAAALTAGFRASEILYTGPGKTDGELDEAIGKGVKTFCVESLTDLQHVGAVALRHGVVADCLLRINSATASATTSIRMTGTPSQFGIDSETLVDAMPELRAVPGTRITGLHFFPLSNARDEASLIGEFRHTIAYAAGLAEETGLTLEFLDIGGGFAHPYGAPGERPVYRELRTELAAALDEHFPHWREGAPRIAVETGRYQTSGAGTLLTRVVNIKVSRGRKFVVIDAGINTFGGMSGLGRLLPVAVEPEYTGSAEATELTDVASLAGPLCTPGDILGREIRLPELAPGDLVTIPNAGAYGVTASLLMFLGRPAPVEVVLKGGKVVSASRLEHHRTPATPG

SEQ ID NO: 20 - protein, 5836 aa, Streptomyces halstedii sp.HC-34
MSPALARSKRMSESENPVSATTAGADETPDTRTALARRLAGLSPAEQEQHLVDMVHRHTVAALQAVAPLTPDQVDVQRPFLELGFDSLAAVDLHKRLTGETGLELPVTVAFDFPTPVLVAEEIRRIAFGIRPAPLAPVVATGNLDDDPIAIIGIGCRFPGGINSAEELWQLVMDEAEVLSGFPTNRGWDVEGIYDPDPGKPGKSYVREGGFLQDAGDFDADFFGISPREALAMDPQQRLVLETAWEAFERAGIDPGSLHGSKAGVFIGAEVHEYGTRVHEAPEGLDGYLMTGNAPSVASGRIAYSLGLEGPAVTIDTACSGSLVALHLAAHSLRQGESSLAIAGGVTVMGNPGMFAAFSRQRGLAADGRCKPFAAAADGTGFSEGVGVFVVERLADARRNGHPVLGIVKGSAINQDGASNGLTAPNGPSQQRLILQALANAGLKPADVDTMEAHGTGTKLGDPIEAQAILATYGQEREEGRPLWLGSIKSNLGHTQAAGGAASLIKMLMGMRHGKLPRSLHIDAPTPHVDWTTGDVALLTETRDWDAPDRPRRAGVSAFGISGTNAHVIIEEAPEFEPSAVEPVAGSGVTPPWVLSARSADALRGQAERLLSFVSTDADVSVVDVAYSLGVSRAGLEHRGVVLGESREELIAALESLASGAEAPGVVTGRVAEGRLAFLFTGQGAQRVGMGRELAAEFPVFAASLEETCDLLDVGLEDQDHSLREVLFAEEGSAEAALLTRTVYAQAALFAVEVALFRLVESFGVVPDFVAGHSVGEIAAAHVAGVFSLEDATMLVAARGRLMDALPEGGTMVAVQATEEDVLALLEGVEDASIAAINGPDAVVVSGTETGVARVVDVLRDRGAKTKRLDVSHAFHSPLMEPMLTEFRRVAEVLEYQAPRIAVVSNVSGSVAGTELATPEYWVTHVREAVRFSAGVRTLLGAGVSSFLEIGPDGVLSGMARGSVSDGADVGCVPVMRRGRGEVREFLTGLSRIAVRGVPVTWEPLIEGARRVELPTYAFQRRWFWLEAGRSTTDALGLGQTAAEHPLVGAVVGLAGGDGVVLTGRVSLHTHPWLADHQVAGVTLLPGTGFVELAVRAGDEVGCGRLEELTLEAPLVVPDRGAVQLQVVVGALEESDVRTVSVYSRAEEDDVWTRHATGFVGAAVDGGLNLEAWPPPGARAVDVSDVYAGLADQGYGYGPVFQGLRSVWRGDGEVFAEVVLPEGAQADASAFGLHPALLDAALHATDYLDPESAVEGTHLPFAWTGVSLHATGASELRVRITATGNDGYALDLADTTGRPVATVQSLVLRPVTEEQLRSAQGGNGPDSLFRVEWAPVTADLTASGAGWAVLGAGAPELAAALTAAGTPGTAYHGTAELSAAVDGGTTPDVVAVQLPSAGAADADLPDAVRATLNTALEFVQNWLADERLAETRLVVVTRDAVTVDTAAGGPDLTVAPVWGLIRSAQAENPGRILLADIDGTDQSRTALAAITAADEPELAFRNGNAYAPRLVRSVSEGGLVPPSDAASWRLDVLSQGTLENLALIPSDADERTLAPGEVRIAVRAAGVNFRDVLVALGMYPTKADIGGEAAGLVLEVGPGVTDFTPGDRVMGLFDTAFGPHAITDHRTLVPMPTGWTYAQAATAPLVFATAYYGLVDLAELRSGESALIHAAAGGVGMAAVQIARHLGAEVYGTASPGKWNALRAAGLDDNHIASSRDTTFEQKFLANSGGRGVDVVLDALTGEFIDASLRLLPRGGRFAEMGKTDVRDPERVAAEYPGVRYRAFDLFEADLDRLREILRELLALFESGALRPLPVRAWDIRKSKDAFRHIAQARHIGKVALTMPTGGLGDGVVLVTGGTGGLGALVARHVVVVHGVRRLVLVSRRGLGAPGAVGLVAELEGLGAVVEVVACDVSDRVALAGVVGGIGSDLSAVVHTAGVVDDGVVGSLSVGRLSSVLGPKADAAWYLHELTVGLDLSAFVLFSSVAGVVDGAGQGNYAAANVFLDGLAVVRRGLGLPGTSVAWGLWEGAGMGAVLGEADVLRMSRSGVLGLSVGEGLGLFDVALGEDVAALVPVRLDLGALRSRVDGVPAVFRSLVRVPVRRSVGVGVSGGGEVSLERRLVVLDAGERERVLLELVRSHVAAVLGYEGAGSIEPGRAFSDIGFDSLSAVELRNRLNGETGLRLPATLIFDYPTPQALAEYVIVALLGEEAANAPALPPTEAPTVTGTDDDPVVIVGMGCRFPGDVRTPEDLWRLVSNGSDAVTPFPDNRAWDIEGIYDPLPGVSGKTYAREGGFLHDAAEFDPEFFGISPREALAMDPQQRLLLETSWEAIERAGIDPNSLRGTQTGVFAGVMQTDYGNGGARLAEDVEGYIANGTLGSIVSGRVSYALGLEGPAVTIDTACSSSLVAMHWAAHALRQGECSLALAGGVTVMSTPETFVDFSLQRGLAPNGRCKAFSADADGTGWGEGVGVLVLERLSDARRNGHQVLAVIRGSALNQDGASNGLTAPNGPSQQRVIRQALVSAGLTTSDVDAVEAHGTGTTLGDPIEAQALLATYGQERDADQPLWLGSVKTNVGHTQAAAGVAGVIKMVLAMRAGVLPKTLHVTEPSPHVDWSAGAVELLTETRDWPETGRPRRAGVSSFGVSGTNAHVIVEQAPAVEEEPTRALPALPVPWSISAKSADALRGQAQALSSFVSAAGDVSVVDVAYSLGVSRAGLEHRGVVVGESRAELLVALESLASGVESPGVVTGRVAEGRLAFLFTGQGAQRVGMGRELAAEFPVFAASLEQTCDLLERAGVAVREVLFAEEGSAEAALLTRTVYAQAALFAVEVALFRLVESFGVVPDFVAGHSVGEIAAAHVAGVFSLEDAVSLVAARGRLMDALPEGGAMVAVQATEEDVLALLEGVEDASIAAINGPDAVVVSGTETGVARVVDVLRERGAKTKRLVVSHAFHSPLMEPMLADFATVVEGLSYKAPAIPVVSGSVVGSELSTPGYWVRHVREAVRFGAGVETLLGAGVSSFLEIGPDGVLSGMARTSVPEGADVECAPLMRRDRAEVREFLTGLSRLAVRGVPVAWESLVEGGRRVELPTYAFQRRRFWLEAGRSVTDASGLGQTAAEHPLVGAVVGLAGGDGVVLTGRVSLHTHPWLADHQVAGVTLLPGTGFVELAVRAGDEVGCGRLEELTLEAPLVVPDRGAVQLQVVVGALETSGVRTVSVYSRVEEESADDTWVRHATGALMAAAEEPGFDLTAWPPAGARAVDVSDAYAGLASQGYGYGPVFQGLTAAWRKGREVFAEITLPEAAQVDASAFGLHPALLDAALHATDYLDQESAEGTHLPFAWSGVSLHAAGASALRVRIVATGKDGYALDLADSTGRPVATVQSLVTRPVTADQLSEASATQHQSLFRMEWTATTAPAATADVRWALLGEPVAELGDLAGFGRTTAYASLADLAAADADTVPDLVVLPLGSRMAEGSDVPEAVRENLGQVLATIQQWNDDSRFDGSRLVVLTHGAVSVSDDESLTALDLAPVWGLVRSAMAETPGRFLLVDTDGTDPSTDALRHLAELQEAEFALREGEIRVPRLARVPVAGAGADITGGLGDGVVLVTGGTGGLGAVVARHAVVVHGVRRLVLVSRRGLGAPGAVGLVAELESLGAVVEVVACDVSDRVALAGVVGGIGSDLSAVVHTAGVVDDGVVGSLSVGRLSSVLGPKADAAWYLHELTVRLDLSAFVLFSSVAGVVDGAGQGNYAAANVFLDGLAVVRRGLGLPGTSVAWGLWEGAGMGAVLGEADVLRMSRSGVLGLSVGEGLGLFDVALGEDVAALVPVRLDLGALRSRVDGVPAVFRSLVRVPVRRSVGVGVSGGGEVSLERRLAVLDAGERERVLLELVRSHVAAVLGYEGAGSIEPGRAFSDIGFDSLSAVELRNRLNGETGLRLPATLIFDYPTPQALADLIQEKTLGLVDAAAATVSTTVSRTDDEPIAIVGMGCRYPGDVRTPEELWRLVASGTDAVSLFPEDRGWNVDSIYDPTPGLSGKTYTREGGFLHDAAEFDAGFFGISPREALAMDPQQRLLLETSWEAMERAGIDPNSLRGTQTGVFAGIMYHDYGSRVTQPSEEVEGYLGNGSAGSIASGRVSYTFGFEGPAVTVDTACSSSLVALHWAAQALRQGECSMALAGGVTVMSTPETFVDFSLQRGLATNGRCKAFSSDADGTGWGEGAGMLLLERLSDARKNGHEVLAVIRGSALNQDGASNGLTAPNGPSQQRVIRQALANAGLSAAEVDAVEAHGTGTKLGDPIEAQALLATYGQERDGDQPLLLGSIKSNVGHTQAAAGVGGVIKMVMAIRNGVLPKTLHVTEPSPHVDWSAGAVELLAEAREWPETGRPRRAGVSSFGISGTNAHVIVEQAPTDQPAPLSEPVGDVPGLPVPWIVSAKSAEALRAQAGRLGSFLGETGVVDVPAVGWSLVRGRSVFAHRAVVVGGEWDELLAGVARVAEGVDDSVVSGVADVSGRRVFVFPGQGSQWVGMAQGLLDSSVVFTERMTECAAALDPLVEWSLLDVVRGVEGAASLERVDVVQPVLWAVMVSLASVWRSVGVVPDAVVGHSQGEIAAAVVGGWLSLVDGARVVALRSLAIREVLAGGGGMVAVQAAEDEVAGWLEGVEGVGIAAVNGPRSVVISGTRAGLDACVELWSGRGTWVKRVPVDYASHSAEVERVRERVLADLASVTGLSGSVPMLSTMTGDWIVEGQVGAGYWVENLRRPVLFADATRRLASEGFGAFVEVSAHPVLVMGIEETIEALRSGAADGAKSGAGEESSSAVVAVGTLRRGEGGWDQFLRSLAGLFVRGAVAPDWESLLGGVRPRVDLPTYAFQRERLWLDAGVVAGDVSGLGQVVVGHPLLGAGVGVAGEGGGVLFTGRLGLGSHPWLGDHAVSGVVLLPGAAFVELVVRAGDEVGCGRLEELTLAAPLVVPERGSVRIQVVVGAGDGSGARSVGVWSSVGDEGVGGEWVCHASGLLTADVGVAPVLGGVWPPVGGVAVDVSGVYEGLALEGYEYGSVFRGLRSVWRRGDEVFAEVALGEGVGVEGFGLHPALLDAALQAAGFGSFVPESEAGSEAGSGGVRLPFSWSGVSLFASGASVGRVRLWPVGGDGFGVELFDGVGMPVARVDALVTREISAGQLGAAAGAGSLVGGESLFRVEWAPVSGVAPASAGVGGCVVVGAGSVLSGFGEVVPDLAAVSAGSADGVGVPGWVLVDVDAWLGADLAVGVVSGEGVPVVARGVVARVLGLVREWLGDERWVSSRLVWVTRGAVGARVLDEVSGVVSSGLWGLVRAAQSEHPDRFALLDLDSATAVDAVRDGVLGLLAAGEPQLVVREGEVLAARLTPAHTTDEPTGQEFGKAATGTVLVTGGTGGLGAVVARHLVTEHGAQRLLLTSRRGINAPGAAELVAELAELGARADVAACDVSDRAALKELLAGVPGDAPLTAVVHAAGVLDDGVIESMTADRLDAVMRPKVDAAWHLHELTADRELDAFVLFSSAAGTLDGAGQSNYAAANVFLDALAQYRRGQGLAGLSLAWGLWGESTGMVGALEGSDLDRIGRSGVRALSSAEGLALFDAAAVLGEPAVLPVALDLGVLRTQPRNQVPAILRGFAAGPTRRTAVTGGPETDQEALTRRLASLSPADRDRFLLDLVRTQVAGVLGYSGPDAIDPQRGFQELGVDSLAAVQIRNRLGAATGVRPPTTLVFDYPTPDAVAGYFKEHLVIEEEDSTAELMREIARLEAAVTSAASSAGGAGLAPAVDRLRAMAAKLADADAQRADEDDPGLESATADELFDILDGELSTD

SEQ ID NO: 21 - DNA, 23 bp, Artificial Sequence
  Primer for PCR (46DH-1)
acsggycsbgccgchttcatcgg

SEQ ID NO: 22 - DNA, 24 bp, Artificial Sequence
  Primer for PCR (46DH-2)
grwrctgrtrsggccgtagttgtt

SEQ ID NO: 23 - DNA, 22 bp, Artificial Sequence
  Primer for PCR (23DH-1)
aggccacccgsagcaactacac

SEQ ID NO: 24 - DNA, 21 bp, Artificial Sequence
  Primer for PCR (23DH-2)
gaascgsccgccctcctcsga

SEQ ID NO: 25 - DNA, 26 bp, Artificial Sequence
  Primer for PCR (vinC-Nterm)
aaggtaccatatgcgcgtcctgatga

SEQ ID NO: 26 - DNA, 26 bp, Artificial Sequence
  Primer for PCR (vinC-Cterm)
cccggatccgctgggcggagtgctac